(19) United States
(12) Patent Application Publication
(10) Pub. No.: US 2007/0128211 A1
(43) Pub. Date: Jun. 7, 2007

(54) Surface proteins of Streptococcus pyogenes

(75) Inventors: Stephen Bruce Olmsted, West Nyack, NY (US); Robert John Zagursky, Victor, NY (US); Elliott Bruce Nickbarg, Belmont, MA (US); Laurie Anne Winter, Brockport, NY (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Wyeth, Madison, NJ (US)

(21) Appl. No.: 11/592,153

(22) Filed: Nov. 3, 2006

Related U.S. Application Data
(62) Division of application No. 10/474,792, filed on May 5, 2004.
(60) Provisional application No. 60/283,358, filed on Apr. 13, 2001.

(51) Int. Cl. A61K 39/02; C07H 21/04; C12P 21/06; C07K 14/315; C07K 16/12; C12N 15/74

(52) U.S. Cl. 424/190.1; 530/350; 530/388.4; 435/69.1; 435/252.3; 435/471; 536/23.7

(57) ABSTRACT

β-hemolytic streptococci polynucleotides, polypeptides, particularly Streptococcus pyogenes polypeptides and polynucleotides, and antibodies of these polypeptides are described. The polynucleotides, polypeptides, and antibodies of the invention can be formulated for use as immunogenic compositions. Also disclosed are methods for immunizing against and reducing β-hemolytic streptococcal infection, and for detecting β-hemolytic streptococci in a biological sample.

### PRIORITY DATA

This is a divisional of U.S. patent application Ser No. 10/474,792 filed Oct. 14, 2003, which is a U.S. national phase under 35 U.S.C. § 371 of International Patent Application No. PCT/US02/11610 filed Apr. 12, 2002, and claims priority under 35 U.S.C. § 119(e) from U.S. Provisional Patent Application No. 60/283,358 filed Apr. 13, 2001, which are incorporated by reference in their entirety.

### FIELD OF THE INVENTION

This invention relates generally to β-hemolytic streptococcal polypeptides and polynucleotides, particularly Streptococcus pyogenes polypeptides and polynucleotides. More specifically, the invention relates to polypeptides of Streptococcus pyogenes which are surface localized, and antibodies of these polypeptides. The invention also relates to nucleotide sequences encoding polypeptides of Streptococcus pyogenes, and expression vectors including these nucleotide sequences. The invention further relates to immunogenic compositions, and methods for immunizing against and reducing β-hemolytic streptococcal infection. The invention also relates to methods of detecting these nucleotides and polypeptides and for detecting β-hemolytic streptococci and Streptococcus pyogenes in a biological sample.

### BACKGROUND OF THE INVENTION

Traditional phenotypic criteria for classification of streptococci include both hemolytic reactions and Lancefield serological groupings. However, with taxonomic advances, it is now known that unrelated species of β-hemolytic (defined as the complete lysis of sheep erythrocytes in agar plates) streptococci may produce identical Lancefield antigens and that strains genetically related at the species level may have heterogeneous Lancefield antigens. In spite of these exceptions to the traditional rules of streptococcal taxonomy, hemolytic reactions and Lancefield serological tests can still be used to divide streptococci into broad categories as a first step in identification of clinical isolates. Ruoff, K. L., R. A. Whiley, and D. Beighton. 1999. Streptococcus. In P. R. Murray, E. J. Baron, M. A. Pfaller, F. C. Tenover, and R. H. Yolken (eds.), Manual of Clinical Microbiology. American Society of Microbiology Press, Washington, D.C.

β-hemolytic isolates with Lancefield group A, C, or G antigen can be subdivided into two groups: large-colony (>0.5 mm in diameter) and small-colony (<0.5 mm in diameter) formers. Large-colony-forming group A (Streptococcus pyogenes), C, and G strains are “pyogenic” streptococci replete with a variety of effective virulence mechanisms. Streptococcus agalactiae (group B) is still identified reliably by its production of Lancefield group B antigen or other phenotypic traits.

A need exists to develop compositions and methods to ameliorate and prevent infections caused by β-hemolytic streptococci, including groups A, B, C and G. Similarity between these species includes not only virulence factors, but also disease manifestations. Included in the latter are pneumonia, arthritis, abscesses, rhinopharyngitis, metritis, puerperal sepsis, neonatal septicemia, wound infections, meningitis, peritonitis, cellulitis, pyoderma, necrotizing fasciitis, toxic shock syndrome, septicemia, infective endocarditis, pericarditis, glomerulonephritis, and osteomyelitis.

Streptococcus pyogenes are gram-positive diplococci that colonize the pharynx and skin of humans, sites that then serve as the primary reservoir for this organism. An obligate parasite, this bacterium is transmitted by either direct contact of respiratory secretions or by hand-to-mouth. The majority of Streptococcus pyogenes infections are relatively mild illnesses, such as pharyngitis or impetigo. Currently, there are anywhere from twenty million to thirty-five million cases of pharyngitis alone in the U.S., costing about $2 billion for physician visits and other related expenses. Additionally, nonsuppurative sequelae such as rheumatic fever, scarlet fever, and glomerulonephritis result from Streptococcus pyogenes infections. Globally, acute rheumatic fever (ARF) is the most common cause of pediatric heart disease (Bibliography entry 1).

From the initial portals of entry, pharynx, and skin, Streptococcus pyogenes can disseminate to other parts of the body where bacteria are not usually found, such as the blood, deep muscle and fat tissue, or the lungs, and can cause invasive infections. Two of the most severe but least common forms of invasive Streptococcus pyogenes disease are necrotizing fasciitis and streptococcal toxic shock syndrome (STSS). Necrotizing fasciitis (described in the media as “flesh-eating bacteria”) is a destructive infection of muscle and fat tissue. STSS is a rapidly progressing infection causing shock and injury to internal organs such as the kidneys, liver, and lungs. Much of this damage is due to a toxemia rather than localized damage due to bacterial growth.

In 1995, invasive Streptococcus pyogenes infections and STSS became mandated reportable diseases. In contrast to the millions of individuals that acquire pharyngitis and impetigo, the U.S. Centers for Disease Control and Prevention (CDC) mandated case reporting indicates that in 1997 there were from 15,000 to 20,000 cases of invasive Streptococcus pyogenes disease in the United States, resulting in over 2,000 deaths (1). Other reports estimate invasive disease to be as high as 10-20 cases per 100,000 individuals per year (62). More specifically, of the 15,000 to 20,000 cases of invasive disease, 1,100 to 1,500 are cases of necrotizing fasciitis and 1,000 to 1,400 are cases of STSS, with a 20% and 60% mortality rate, respectively. Also included in serious invasive disease are cases of myositis, which carries a fatality rate of 80% to 100%. An additional 10% to 15% of individuals with other forms of invasive group A streptococcal disease die. These numbers have increased since case reporting was initiated in 1995 and reflect a general trend that has occurred over the past decade or two. Additionally, it is commonly agreed that the stringency of the case definitions results in lower and, thus, misleading numbers, in that many cases are successfully resolved due to early diagnosis and treatment before the definition has been met.

While Streptococcus pyogenes remains exquisitely sensitive to penicillin and its derivatives, treatment does not necessarily eradicate the organism. Approximately 5% to 20% of the human population remain carriers depending on the season (62), despite antibiotic therapy. The reasons for this are not totally clear and may involve a variety of mechanisms. In cases of serious invasive infections, treatment often requires aggressive surgical intervention. For those cases involving STSS or related disease, clindamycin (a protein synthesis inhibitor) is the preferred antibiotic as it penetrates tissues well and prevents exotoxin production. There are reports of some resistance to tetracycline, sulfa, and most recently, erythromycin. Clearly, there remains a need for compositions to prevent and treat β-hemolytic infection.

Numerous virulence factors have been identified for Streptococcus pyogenes, some secreted and some surface localized. Although it is encapsulated, the capsule is composed of hyaluronic acid and is not suitable as a candidate antigen for inclusion in immunogenic compositions, since it is commonly expressed by mammalian cells and is nonimmunogenic (14). The T antigen and Group Carbohydrate are other candidates, but may also elicit cross-reactive antibodies to heart tissue. Lipoteichoic acid is present on the surface of Streptococcus pyogenes, but raises safety concerns similar to LPS.

The most abundant surface proteins fall into a family of proteins referred to as M or “M-like” proteins because of their structural similarity. While members of this class have similar biological roles in inhibiting phagocytosis, they each have unique substrate binding properties. The best characterized protein of this family is the helical M protein. Antibodies directed to homologous M strains have been shown to be opsonic and protective (12, 13, 16). Complicating the use of M protein as a candidate antigen is the fact that there have been approximately 100 different serotypes of M protein identified with several more untyped. Typically, the Class I M serotypes, exemplified by serotypes M1, M3, M6, M12, and M18, are associated with pharyngitis, scarlet fever, and rheumatic fever and do not express immunoglobulin binding proteins. Class II M serotypes, such as M2 and M49, are associated with the more common localized skin infections and the sequelae glomerulonephritis, and do express immunoglobulin binding proteins (54). It is important to note that there is little, if any, heterologous cross-reactivity of antibodies to M serotypes. Equally important is the role these antibodies play in rheumatic fever. Specific regions of M protein elicit antibodies that cross react with host heart tissue, causing or at least correlating with cellular damage (11, 57).

M and M-like proteins belong to a large family of surface localized proteins that are defined by the sortase-targeted LPXTG motif (38, 64). This motif, located near the carboxy-terminus of the protein, is first cleaved by sortase between the threonine and glycine residues of the LPXTG motif. Once cleaved, the protein is covalently attached via the carboxyl of threonine to a free amide group of the amino acid cross-bridge in the peptidoglycan, thus permanently attaching the protein to the surface of the bacterial cell. Included in this family of sortase-targeted proteins are the C5a peptidase (6, 7), adhesins for fibronectin (9, 19, 23, 24), vitronectin, and type IV collagen, and other M-like proteins that bind plasminogen, IgA, IgG, and albumin (31).

Numerous secreted proteins have been described, several of which are considered to be toxins. Most Streptococcus pyogenes isolates from cases of serious invasive disease and streptococcal toxic shock syndrome (STSS) produce streptococcal pyrogenic exotoxins (SPE) A and C (8). Other pyrogenic exotoxins have also been identified in the genomic Streptococcus pyogenes sequence completed at the University of Oklahoma, submitted to GenBank and assigned accession number AE004092, and have been characterized (55). Other toxins such as Toxic Shock Like Syndrome toxin, Streptococcal Superantigen (58), and Mitogenic Factor (66) play lesser-defined roles in disease. Streptolysin O could also be considered a possible candidate antigen, because it causes the release of IL-β release. In addition, a variety of secreted enzymes have also been identified that include the Cysteine protease (35, 37), Streptokinase (26, 48), and Hyaluronidase (27, 28).

Given the number of known virulence factors produced by Streptococcus pyogenes, it is clear that an important characteristic for a successful β-hemolytic streptococcal immunogenic composition would be its ability to stimulate a response that would prevent or limit colonization early in the infection process. This protective response would either block adherence and/or enhance the clearance of cells through opsonophagocytosis. Antibodies to M protein have been shown to be opsonic and provide a mechanism to overcome the anti-phagocytic properties of the protein (30) in much the same way that anti-serotype B capsular antibodies have demonstrated protection from disease caused by Haemophilus influenzae B (36). In addition, antibodies specific to Protein F have been shown to block adherence and internalization by tissue culture cells (43).

There remains a need to further identify immunogenic compositions, and methods for the prevention or amelioration of β-hemolytic streptococcal colonization or infection. There also remains a need to further identify surface proteins of Streptococcus pyogenes and polynucleotides that encode Streptococcus pyogenes polypeptides. Also, there remains a need for methods of detecting β-hemolytic streptococci and Streptococcus pyogenes colonization or infection.

### SUMMARY OF THE INVENTION

To meet these and other needs, and in view of its purposes, the present invention provides compositions and methods for the prevention or amelioration of β-hemolytic streptococcal colonization or infection. The invention also provides Streptococcus pyogenes polypeptides and polynucleotides, recombinant materials, and methods for their production. Another aspect of the invention relates to methods for using such Streptococcus pyogenes polypeptides and polynucleotides.

The polypeptides of the invention include isolated polypeptides comprising at least one of an amino acid sequence of any of even numbered SEQ ID NOS: 2-668. The invention also includes amino acid sequences that have at least 70% identity to any of an amino acid sequence of even numbered SEQ ID NOS: 2-668, and mature polypeptides of the amino acid sequences any of even numbered SEQ ID NOS: 2-668. The invention further includes immunogenic fragments and biological equivalents of these polypeptides. Also provided are antibodies that immunospecifically bind to the polypeptides of the invention.

The polynucleotides of the invention include isolated polynucleotides that comprise nucleotide sequences that encode a polypeptide of the invention. These polynucleotides include isolated polynucleotides comprising at least one of a nucleotide sequence of any of odd numbered SEQ ID NOS: 1-667, and also include other nucleotide sequences that, as a result of the degeneracy of the genetic code, also encode a polypeptide of the invention. The invention also includes isolated polynucleotides comprising a nucleotide sequence that has at least 70% identity to a nucleotide sequence that encodes a polypeptide of the invention, and isolated polynucleotides comprising a nucleotide sequences that has at least 70% identity to a nucleotide sequence any of odd numbered SEQ ID NOS: 1-667. In addition, the isolated polynucleotides of the invention include nucleotide sequences that hybridize under stringent hybridization conditions to a nucleotide sequence that encodes a polypeptide of the invention, nucleotide sequences that hybridize under stringent hybridization conditions to a nucleotide sequence of any of odd numbered SEQ ID NOS: 1-667, and nucleotide sequences that are fully complementary to these polynucleotides. Furthermore, the invention includes expression vectors and host cells comprising these polynucleotides.

The invention further provides methods for producing the polypeptides of the invention. In one embodiment, the method comprises the steps of (a) culturing a recombinant host cell of the invention under conditions suitable to produce a polypeptide of the invention and (b) recovering the polypeptide from the culture.

The invention also provides immunogenic compositions. In one embodiment, the immunogenic compositions comprise an immunogenic amount of at least one component which comprises a polypeptide of the invention in an amount effective to prevent or ameliorate a β-hemolytic streptococcal colonization or infection in a susceptible mammal. The component may comprise the polypeptide itself, or may comprise the polypeptide and any other substance (e.g., one or more chemical agents, proteins, etc.) that can aid in the prevention and/or amelioration of β-hemolytic streptococcal colonization or infection. These immunogenic compositions can further comprise at least a portion of the polypeptide, optionally conjugated or linked to a peptide, polypeptide, or protein, or to a polysaccharide. In another embodiment, the immunogenic compositions comprise an immunogenic amount of a component which comprises a polynucleotide of the invention, the component being in an amount effective to prevent or ameliorate a β-hemolytic streptococcal colonization or infection in a susceptible mammal. The component may comprise the polynucleotide itself, or may comprise the polynucleotide and any other substance (e.g., one or more chemical agents, proteins, etc.) that can aid in the prevention and/or amelioration of β-hemolytic streptococcal colonization or infection. In yet another embodiment, the immunogenic compositions comprise a vector that comprises a polynucleotide of the invention. The immunogenic compositions of the invention can also include an effective amount of an adjuvant.

The invention also includes methods of protecting a susceptible mammal against β-hemolytic streptococcal colonization or infection. In one embodiment, the method comprises administering to a mammal an effective amount of an immunogenic composition comprising an immunogenic amount of a polypeptide of the invention, which amount is effective to prevent or ameliorate β-hemolytic streptococcal colonization or infection in the susceptible mammal. In another embodiment, the method comprises administering to the mammal an effective amount of an immunogenic composition comprising a polynucleotide of the invention, which amount is effective to prevent or ameliorate β-hemolytic streptococcal colonization or infection in the susceptible mammal. The immunogenic compositions of the invention can be administered by any conventional route, for example, by subcutaneous or intramuscular injection, oral ingestion, or intranasally.

The invention further includes compositions and methods for reducing at least one of the number and the growth of β-hemolytic streptococci in a mammal having a β-hemolytic streptococcal colonization or infection. In one embodiment, the composition comprises an antibody of the invention. In another embodiment, the composition comprises an antisense oligonucleotide capable of blocking expression of a nucleotide sequence encoding a polypeptide of the invention.

Also provided are methods for reducing side effects caused by β-hemolytic streptococcal infection in a mammal. In one embodiment, the method comprises administering to the mammal an effective amount of a composition comprising an antibody of the invention, which amount is effective to reduce at least one of the number of and the growth of β-hemolytic streptococci in the mammal. In another embodiment, the method comprises administering to the mammal an effective amount of a composition comprising an antisense oligonucleotide capable of blocking expression of a nucleotide sequence encoding a polypeptide of the invention, which amount is effective to reduce at least one of the number of and the growth of β-hemolytic streptococci in the mammal.

Also provided are methods for detecting and/or identifying β-hemolytic streptococci in a biological sample. In one embodiment, the method comprises (a) contacting the biological sample with a polynucleotide of the invention under conditions that permit hybridization of complementary base pairs and (b) detecting the presence of hybridization complexes in the sample, wherein the detection of hybridization complexes indicates the presence of β-hemolytic streptococci in the biological sample. In another embodiment, the method comprises (a) contacting the biological sample with an antibody of the invention under conditions suitable for the formation of immune complexes and (b) detecting the presence of immune complexes in the sample, wherein the detection of immune complexes indicates the presence of β-hemolytic streptococci in the biological sample. In yet another embodiment, the method comprises (a) contacting the biological sample with a polypeptide of the invention under conditions suitable for the formation of immune complexes and (b) detecting the presence of immune complexes in the sample, wherein the detection of immune complexes indicates the presence of antibodies to β-hemolytic streptococci in the biological sample.

The invention further provides immunogenic compositions. In one embodiment, the immunogenic composition comprises at least one polypeptide of the invention. In another embodiment, the immunogenic composition comprises at least one polynucleotide of the invention. In yet another embodiment, the immunogenic composition comprises at least one antibody of the invention.

Also provided is an isolated polynucleotide comprising a nucleotide sequence that has at least 70% identity to a nucleotide sequence that encodes a polypeptide of the invention, the polynucleotide being identified by the steps comprising (a) obtaining a first and second PCR primer derived from a nucleotide that encodes a mature polypeptide of any of SEQ ID NOS: 2-668, wherein the first and second primers are capable of initiating nucleic acid synthesis in an outward manner under PCR conditions, and wherein the first primer is capable of being extended in an antisense direction and the second primer is capable of being extended in a sense direction and (b) combining the first and second PCR primer with a cDNA library that contains the polynucleotide under PCR conditions suitable for synthesizing the nucleotide sequence from the first and second primers.

Also provided is a method for extending a polynucleotide of the invention using polymerase chain reaction (PCR), the method comprising the steps of (a) obtaining a first and second PCR primer derived from the polynucleotide, wherein the first and second PCR primers are capable of initiating nucleic acid synthesis in an outward manner under PCR conditions, and wherein the first PCR primer is capable of being extended in an antisense direction and the second PCR primer is capable of being extended in a sense direction and (b) combining the first and second PCR primers with the polynucleotide contained in a cDNA library under PCR conditions suitable for synthesizing nucleotide sequences from the first and second PCR primers, thereby extending the polynucleotide.

It is to be understood that the foregoing general description and the following detailed description are exemplary, but are not restrictive, of the invention.

### BRIEF DESCRIPTION OF THE DRAWINGS

FIG. 1 depicts a graphical representation of open reading frame (ORF) identification.

FIG. 2 depicts a low-voltage scanning electron micrograph (LV-SEM) of Streptococcus pyogenes after digestion with trypsin, wherein cell integrity is maintained and an even monolayer is present. The bar equals 1 μm.

FIG. 3 depicts a LV-SEM of Streptococcus pyogenes before and after digestion with trypsin. Panel A (the left panel) shows cells before tryptic digestion, wherein the cells are larger and display surface material. Panel B (the right panel) shows cells after digestion, wherein the cells are smaller and appear devoid of any surface proteins. The bars equal 1 μm.

FIG. 4 depicts a LV-SEM of Streptococcus pyogenes expressing protein encoded by ORF 218.

FIG. 5 depicts a LV-SEM of Streptococcus pyogenes expressing protein encoded by ORF 554.

FIG. 6 depicts a LV-SEM of Streptococcus pyogenes expressing protein encoded by ORF 1191.

FIG. 7 depicts a LV-SEM of Streptococcus pyogenes expressing protein encoded by ORF 2064.

FIG. 8 depicts a LV-SEM of Streptococcus pyogenes expressing protein encoded by ORF 2601.

FIG. 9 depicts a LV-SEM of Streptococcus pyogenes expressing protein encoded by ORF 1316.

FIG. 10 depicts a LV-SEM of Streptococcus pyogenes expressing protein encoded by ORF 1224.

FIG. 11 depicts PCR analysis of several Streptococcus pyogenes strains to illustrate gene conservation across the strains.

FIG. 12 depicts quantitative PCR analysis of selected Streptococcus pyogenes ORFs to demonstrate that all ORFs tested are transcribed in vitro and in vivo.

FIG. 13 depicts a dot blot showing reactivity of human serum with the ORF gene products.

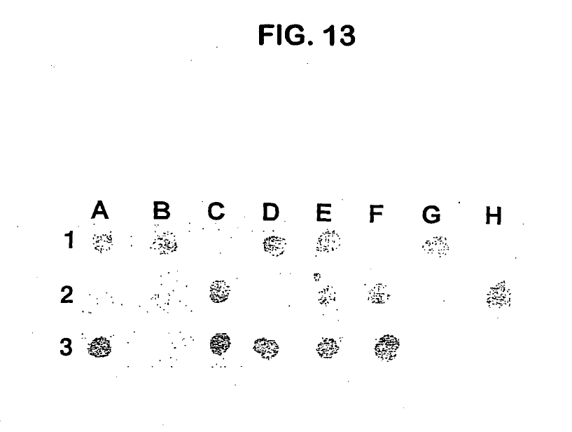

FIG. 14 depicts ability of SPE I to induce rabbit splenocyte proliferation compared to other SPEs.

FIG. 15 depicts human T cell receptor stimulation profile induced by SPE I (black bars) compared to stimulation by anti CD3 antibodies (open bars).

### DETAILED DESCRIPTION OF THE INVENTION

The present invention provides compositions and methods to ameliorate and prevent infections caused by all β-hemolytic streptococci, including groups A, B, C and G. To identify polynucleotides and polypeptides useful for the amelioration and prevention of infections caused by β-hemolytic streptococci, two strategies, a genomic approach and a proteomic approach, were used to identify surface localized, Streptococcus pyogenes proteins.

The genomic approach included an extensive genomic analysis in silico of the Streptococcus pyogenes genome using several algorithms designed to identify and characterize genes that would encode surface localized proteins. The proteomic approach was undertaken to identify proteins present on the surface of Streptococcus pyogenes. Reliance on both approaches was important to overcome the deficiencies of each approach. Genomic mining provides the genetic capabilities, but gives little information as to the actual phenotypic expression. Conversely, proteomic analysis identifies actual proteins localized to the surface of the cell, but protein expression may be regulated and the specific conditions under which the bacterial cells are cultured may influence the set of proteins identified.

The results of the genomic and proteomic approaches were combined and the ORFs of interest were categorized into one of four groups: (i) ORFs encoding surface localized proteins identified by proteomics (Table I, odd numbered SEQ ID NOS: 1-147); (ii) ORFs encoding putative lipoproteins (Table II, odd numbered SEQ ID NOS: 149-181, 669); (iii) ORFs encoding putative polypeptides containing a LPXTG motif (Table III, odd numbered SEQ ID NOS: 183-187); and (iv) ORFs encoding other putative surface localized polypeptides (Table IV, odd numbered SEQ ID NOS: 189-667). The ORFs contained in Tables I-IV are non-redundant, i.e., the ORFs listed in Tables I-IV each appear once though many ORFs possess characteristics that match another table. Thus, for example, there are ORFs listed in Table I (ORFs encoding surface localized proteins identified by proteomics) that could also be classified in one or more of Tables II-IV, but are not included in those tables.

TABLE I
Open Reading Frames (ORFs) encoding surface
localized proteins identified by proteomics

SEQ ID NO: 1 (ORF 66)
SEQ ID NO: 3 (ORF 102)
SEQ ID NO: 5 (ORF 145)
SEQ ID NO: 7 (ORF 232)
SEQ ID NO: 9 (ORF 238)
SEQ ID NO: 11 (ORF 436)
SEQ ID NO: 13 (ORF 516)
SEQ ID NO: 15 (ORF 554)
SEQ ID NO: 17 (ORF 589)
SEQ ID NO: 19 (ORF 661)
SEQ ID NO: 21 (ORF 668)
SEQ ID NO: 23 (ORF 678)
SEQ ID NO: 25 (ORF 704)
SEQ ID NO: 27 (ORF 743)
SEQ ID NO: 29 (ORF 825)
SEQ ID NO: 31 (ORF 850)
SEQ ID NO: 33 (ORF 934)
SEQ ID NO: 35 (ORF 993)
SEQ ID NO: 37 (ORF 1036)
SEQ ID NO: 39 (ORF 1140)
SEQ ID NO: 41 (ORF 1157)
SEQ ID NO: 43 (ORF 1191)
SEQ ID NO: 45 (ORF 1218)
SEQ ID NO: 47 (ORF 1224)
SEQ ID NO: 49 (ORF 1234)
SEQ ID NO: 51 (ORF 1237)
SEQ ID NO: 53 (ORF 1238)
SEQ ID NO: 55 (ORF 1253)
SEQ ID NO: 57 (ORF 1284)
SEQ ID NO: 59 (ORF 1316)
SEQ ID NO: 61 (ORF 1330)
SEQ ID NO: 63 (ORF 1358)
SEQ ID NO: 65 (ORF 1487)
SEQ ID NO: 67 (ORF 1495)
SEQ ID NO: 69 (ORF 1557)
SEQ ID NO: 71 (ORF 1638)
SEQ ID NO: 73 (ORF 1650)
SEQ ID NO: 75 (ORF 1654)
SEQ ID NO: 77 (ORF 1659)
SEQ ID NO: 79 (ORF 1698)
SEQ ID NO: 81 (ORF 1788)
SEQ ID NO: 83 (ORF 1794)
SEQ ID NO: 85 (ORF 1816)
SEQ ID NO: 87 (ORF 1818)
SEQ ID NO: 89 (ORF 1819)
SEQ ID NO: 91 (ORF 1850)
SEQ ID NO: 93 (ORF 1854)
SEQ ID NO: 95 (ORF 1878)
SEQ ID NO: 97 (ORF 1902)
SEQ ID NO: 99 (ORF 1943)
SEQ ID NO: 101 (ORF 1975)
SEQ ID NO: 103 (ORF 2019)
SEQ ID NO: 105 (ORF 2064)
SEQ ID NO: 107 (ORF 2086)
SEQ ID NO: 109 (ORF 2106)
SEQ ID NO: 111 (ORF 2116)
SEQ ID NO: 113 (ORF 2120)
SEQ ID NO: 115 (ORF 2123)
SEQ ID NO: 117 (ORF 2202)
SEQ ID NO: 119 (ORF 2214)
SEQ ID NO: 121 (ORF 2330)
SEQ ID NO: 123 (ORF 2354)
SEQ ID NO: 125 (ORF 2377)
SEQ ID NO: 127 (ORF 2379)
SEQ ID NO: 129 (ORF 2387)
SEQ ID NO: 131 (ORF 2417)
SEQ ID NO: 133 (ORF 2420)
SEQ ID NO: 135 (ORF 2422)
SEQ ID NO: 137 (ORF 2450)
SEQ ID NO: 139 (ORF 2459)
SEQ ID NO: 141 (ORF 2477)
SEQ ID NO: 143 (ORF 2586)
SEQ ID NO: 145 (ORF 2593)
SEQ ID NO: 147 (ORF 2601)

TABLE II
Open Reading Frames (ORFs) encoding putative lipoproteins

SEQ ID NO: 149 (ORF 68)
SEQ ID NO: 151 (ORF 309)
SEQ ID NO: 153 (ORF 347)
SEQ ID NO: 155 (ORF 540)
SEQ ID NO: 157 (ORF 601)
SEQ ID NO: 159 (ORF 664)
SEQ ID NO: 161 (ORF 685)
SEQ ID NO: 163 (ORF 729)
SEQ ID NO: 165 (ORF 747)
SEQ ID NO: 167 (ORF 1202)
SEQ ID NO: 169 (ORF 1723)
SEQ ID NO: 171 (ORF 1755)
SEQ ID NO: 173 (ORF 1789)
SEQ ID NO: 175 (ORF 1882)
SEQ ID NO: 177 (ORF 1918)
SEQ ID NO: 179 (ORF 1983)
SEQ ID NO: 181 (ORF 2452)
SEQ ID NO: 669 (ORF 1664)

TABLE III
Open Reading Frames (ORFs) encoding putative
polypeptides containing a LPXTG motif

SEQ ID NO: 183 (ORF 433)
SEQ ID NO: 185 (ORF 967)
SEQ ID NO: 187 (ORF 2497)

TABLE IV
Open Reading Frames (ORFs) encoding other
putative surface localized polypeptides

SEQ ID NO: 189 (ORF 4)
SEQ ID NO: 191 (ORF 5)
SEQ ID NO: 193 (ORF 11)
SEQ ID NO: 195 (ORF 17)
SEQ ID NO: 197 (ORF 18)
SEQ ID NO: 199 (ORF 20)
SEQ ID NO: 201 (ORF 25)
SEQ ID NO: 203 (ORF 49)
SEQ ID NO: 205 (ORF 64)
SEQ ID NO: 207 (ORF 65)
SEQ ID NO: 209 (ORF 67)
SEQ ID NO: 211 (ORF 69)
SEQ ID NO: 213 (ORF 72)
SEQ ID NO: 215 (ORF 73)
SEQ ID NO: 217 (ORF 75)
SEQ ID NO: 219 (ORF 98)
SEQ ID NO: 221 (ORF 99)
SEQ ID NO: 223 (ORF 130)
SEQ ID NO: 225 (ORF 133)
SEQ ID NO: 227 (ORF 141)
SEQ ID NO: 229 (ORF 151)
SEQ ID NO: 231 (ORF 165)
SEQ ID NO: 233 (ORF 172)
SEQ ID NO: 235 (ORF 184)
SEQ ID NO: 237 (ORF 189)
SEQ ID NO: 239 (ORF 199)
SEQ ID NO: 241 (ORF 209)
SEQ ID NO: 243 (ORF 218)
SEQ ID NO: 245 (ORF 220)
SEQ ID NO: 247 (ORF 223)
SEQ ID NO: 249 (ORF 227)
SEQ ID NO: 251 (ORF 241)
SEQ ID NO: 253 (ORF 252)
SEQ ID NO: 255 (ORF 264)
SEQ ID NO: 257 (ORF 265)
SEQ ID NO: 259 (ORF 291)
SEQ ID NO: 261 (ORF 292)
SEQ ID NO: 263 (ORF 306)
SEQ ID NO: 265 (ORF 307)
SEQ ID NO: 267 (ORF 313)
SEQ ID NO: 269 (ORF 350)
SEQ ID NO: 271 (ORF 352)
SEQ ID NO: 273 (ORF 353)
SEQ ID NO: 275 (ORF 368)
SEQ ID NO: 277 (ORF 401)
SEQ ID NO: 279 (ORF 405)
SEQ ID NO: 281 (ORF 421)
SEQ ID NO: 283 (ORF 491)
SEQ ID NO: 285 (ORF 510)
SEQ ID NO: 287 (ORF 511)
SEQ ID NO: 289 (ORF 519)
SEQ ID NO: 291 (ORF 523)
SEQ ID NO: 293 (ORF 535)
SEQ ID NO: 295 (ORF 551)
SEQ ID NO: 297 (ORF 567)
SEQ ID NO: 299 (ORF 570)
SEQ ID NO: 301 (ORF 594)
SEQ ID NO: 303 (ORF 597)
SEQ ID NO: 305 (ORF 602)
SEQ ID NO: 307 (ORF 613)
SEQ ID NO: 309 (ORF 627)
SEQ ID NO: 311 (ORF 639)
SEQ ID NO: 313 (ORF 644)
SEQ ID NO: 315 (ORF 650)
SEQ ID NO: 317 (ORF 653)
SEQ ID NO: 319 (ORF 665)
SEQ ID NO: 321 (ORF 670)
SEQ ID NO: 323 (ORF 671)
SEQ ID NO: 325 (ORF 672)
SEQ ID NO: 327 (ORF 674)
SEQ ID NO: 329 (ORF 676)
SEQ ID NO: 331 (ORF 688)
SEQ ID NO: 333 (ORF 699)
SEQ ID NO: 335 (ORF 702)
SEQ ID NO: 337 (ORF 705)
SEQ ID NO: 339 (ORF 706)
SEQ ID NO: 341 (ORF 721)
SEQ ID NO: 343 (ORF 731)
SEQ ID NO: 345 (ORF 733)
SEQ ID NO: 347 (ORF 737)
SEQ ID NO: 349 (ORF 741)
SEQ ID NO: 351 (ORF 754)
SEQ ID NO: 353 (ORF 774)
SEQ ID NO: 355 (ORF 783)
SEQ ID NO: 357 (ORF 788)
SEQ ID NO: 359 (ORF 805)
SEQ ID NO: 361 (ORF 814)
SEQ ID NO: 363 (ORF 818)
SEQ ID NO: 365 (ORF 844)
SEQ ID NO: 367 (ORF 848)
SEQ ID NO: 369 (ORF 858)
SEQ ID NO: 371 (ORF 859)
SEQ ID NO: 373 (ORF 860)
SEQ ID NO: 375 (ORF 871)
SEQ ID NO: 377 (ORF 877)
SEQ ID NO: 379 (ORF 896)
SEQ ID NO: 381 (ORF 908)
SEQ ID NO: 383 (ORF 909)
SEQ ID NO: 385 (ORF 910)
SEQ ID NO: 387 (ORF 920)
SEQ ID NO: 389 (ORF 921)
SEQ ID NO: 391 (ORF 926)
SEQ ID NO: 393 (ORF 928)
SEQ ID NO: 395 (ORF 929)
SEQ ID NO: 397 (ORF 933)
SEQ ID NO: 399 (ORF 952)
SEQ ID NO: 401 (ORF 961)
SEQ ID NO: 403 (ORF 975)
SEQ ID NO: 405 (ORF 983)
SEQ ID NO: 407 (ORF 991)
SEQ ID NO: 409 (ORF 1015)
SEQ ID NO: 411 (ORF 1018)
SEQ ID NO: 413 (ORF 1020)
SEQ ID NO: 415 (ORF 1021)
SEQ ID NO: 417 (ORF 1026)
SEQ ID NO: 419 (ORF 1058)
SEQ ID NO: 421 (ORF 1110)
SEQ ID NO: 423 (ORF 1132)
SEQ ID NO: 425 (ORF 1152)
SEQ ID NO: 427 (ORF 1156)
SEQ ID NO: 429 (ORF 1188)
SEQ ID NO: 431 (ORF 1200)
SEQ ID NO: 433 (ORF 1203)
SEQ ID NO: 435 (ORF 1205)
SEQ ID NO: 437 (ORF 1210)
SEQ ID NO: 439 (ORF 1216)
SEQ ID NO: 441 (ORF 1228)
SEQ ID NO: 443 (ORF 1231)
SEQ ID NO: 445 (ORF 1265)
SEQ ID NO: 447 (ORF 1267)
SEQ ID NO: 449 (ORF 1269)
SEQ ID NO: 451 (ORF 1272)
SEQ ID NO: 453 (ORF 1275)
SEQ ID NO: 455 (ORF 1292)
SEQ ID NO: 457 (ORF 1300)
SEQ ID NO: 459 (ORF 1310)
SEQ ID NO: 461 (ORF 1311)
SEQ ID NO: 463 (ORF 1318)
SEQ ID NO: 465 (ORF 1321)
SEQ ID NO: 467 (ORF 1362)
SEQ ID NO: 469 (ORF 1395)
SEQ ID NO: 471 (ORF 1497)
SEQ ID NO: 473 (ORF 1500)
SEQ ID NO: 475 (ORF 1512)
SEQ ID NO: 477 (ORF 1513)
SEQ ID NO: 479 (ORF 1525)
SEQ ID NO: 481 (ORF 1527)
SEQ ID NO: 483 (ORF 1548)
SEQ ID NO: 485 (ORF 1573)
SEQ ID NO: 487 (ORF 1585)
SEQ ID NO: 489 (ORF 1586)
SEQ ID NO: 491 (ORF 1593)
SEQ ID NO: 493 (ORF 1608)
SEQ ID NO: 495 (ORF 1661)
SEQ ID NO: 497 (ORF 1667)
SEQ ID NO: 499 (ORF 1671)
SEQ ID NO: 501 (ORF 1672)
SEQ ID NO: 503 (ORF 1678)
SEQ ID NO: 505 (ORF 1680)
SEQ ID NO: 507 (ORF 1681)
SEQ ID NO: 509 (ORF 1682)
SEQ ID NO: 511 (ORF 1683)
SEQ ID NO: 513 (ORF 1720)
SEQ ID NO: 515 (ORF 1725)
SEQ ID NO: 517 (ORF 1726)
SEQ ID NO: 519 (ORF 1732)
SEQ ID NO: 521 (ORF 1736)
SEQ ID NO: 523 (ORF 1771)
SEQ ID NO: 525 (ORF 1772)
SEQ ID NO: 527 (ORF 1775)
SEQ ID NO: 529 (ORF 1776)
SEQ ID NO: 531 (ORF 1777)
SEQ ID NO: 533 (ORF 1783)
SEQ ID NO: 535 (ORF 1785)
SEQ ID NO: 537 (ORF 1786)
SEQ ID NO: 539 (ORF 1814)
SEQ ID NO: 541 (ORF 1820)
SEQ ID NO: 543 (ORF 1828)
SEQ ID NO: 545 (ORF 1833)
SEQ ID NO: 547 (ORF 1834)
SEQ ID NO: 549 (ORF 1839)
SEQ ID NO: 551 (ORF 1873)
SEQ ID NO: 553 (ORF 1875)
SEQ ID NO: 555 (ORF 1876)
SEQ ID NO: 557 (ORF 1888)
SEQ ID NO: 559 (ORF 1909)
SEQ ID NO: 561 (ORF 1917)
SEQ ID NO: 563 (ORF 1931)
SEQ ID NO: 565 (ORF 1970)
SEQ ID NO: 567 (ORF 1972)
SEQ ID NO: 569 (ORF 1979)
SEQ ID NO: 571 (ORF 1987)
SEQ ID NO: 573 (ORF 1993)
SEQ ID NO: 575 (ORF 2013)
SEQ ID NO: 577 (ORF 2014)
SEQ ID NO: 579 (ORF 2015)
SEQ ID NO: 581 (ORF 2020)
SEQ ID NO: 583 (ORF 2023)
SEQ ID NO: 585 (ORF 2046)
SEQ ID NO: 587 (ORF 2048)
SEQ ID NO: 589 (ORF 2050)
SEQ ID NO: 591 (ORF 2069)
SEQ ID NO: 593 (ORF 2070)
SEQ ID NO: 595 (ORF 2091)
SEQ ID NO: 597 (ORF 2148)
SEQ ID NO: 599 (ORF 2170)
SEQ ID NO: 601 (ORF 2201)
SEQ ID NO: 603 (ORF 2222)
SEQ ID NO: 605 (ORF 2231)
SEQ ID NO: 607 (ORF 2236)
SEQ ID NO: 609 (ORF 2240)
SEQ ID NO: 611 (ORF 2245)
SEQ ID NO: 613 (ORF 2247)
SEQ ID NO: 615 (ORF 2250)
SEQ ID NO: 617 (ORF 2258)
SEQ ID NO: 619 (ORF 2266)
SEQ ID NO: 621 (ORF 2273)
SEQ ID NO: 623 (ORF 2289)
SEQ ID NO: 625 (ORF 2291)
SEQ ID NO: 627 (ORF 2300)
SEQ ID NO: 629 (ORF 2319)
SEQ ID NO: 631 (ORF 2342)
SEQ ID NO: 633 (ORF 2391)
SEQ ID NO: 635 (ORF 2398)
SEQ ID NO: 637 (ORF 2399)
SEQ ID NO: 639 (ORF 2411)
SEQ ID NO: 641 (ORF 2414)
SEQ ID NO: 643 (ORF 2428)
SEQ ID NO: 645 (ORF 2429)
SEQ ID NO: 647 (ORF 2437)
SEQ ID NO: 649 (ORF 2457)
SEQ ID NO: 651 (ORF 2458)
SEQ ID NO: 653 (ORF 2473)
SEQ ID NO: 655 (ORF 2482)
SEQ ID NO: 657 (ORF 2488)
SEQ ID NO: 659 (ORF 2508)
SEQ ID NO: 661 (ORF 2521)
SEQ ID NO: 663 (ORF 2534)
SEQ ID NO: 665 (ORF 2562)
SEQ ID NO: 667 (ORF 2583)

Genomic Approach

The availability of complete bacterial genome sequences is currently playing an important role in the identification of immunogenic composition candidates through genomics, transcriptional profiling, and proteomics, coupled with the information processing capabilities of bioinformatics (39-41, 53, 60, 65).

The genomic approach began by identifying open reading frames (ORFs) in an unannotated sequence of Streptococcus pyogenes downloaded from the website of the University of Oklahoma. This genomic sequence was reported as being submitted to GenBank and assigned accession number AE004092. Strain M1 GAS was reported as being submitted to the ATCC and given accession number ATCC 700294.

An ORF is defined herein as having one of three potential start site codons, ATG, GTG, or TTG, and one of three potential stop codons, TAA, TAG, or TGA. Using this definition of an ORF, the Streptococcus pyogenes genome was analyzed to identify ORFs using three ORF finder algorithms, GLIMMER (59), GeneMark (34), and an algorithm developed by inventor's assignee. There were 736 ORFs commonly identified by all three algorithms. The difference in results between the different ORF finders is primarily due to the particular start codons used by each program, however, Glimmer also incorporates some evaluation for a Shine-Dalgamo box. All ORFs with common stop codons were given the same ORF designation and were treated as if they were the same ORF.

In order to evaluate the accuracy of the ORFs determined, a discrete mathematical cosine function, known in the art as a discrete cosine transformation (DiCTion), was employed to assign a score for each ORF. An ORF with a DiCTion score >1.5 was considered to have a high probability of encoding a protein product. The minimum length of an ORF predicted by the three ORF finding algorithms was set to 225 nucleotides (including stop codon) which would encode a protein of 74 amino acids.

As a final search for remnants of ORFs, all noncoding regions >75 nucleotides were searched against public protein databases using tBLASTn to identify regions of genes that contained frameshifts (42) or fragments of genes that might have a role in causing antigenic variation (21). These remnant ORFs were added to the ORF hits.

A graphical analysis program developed by inventor's assignee was used to show all six reading frames and the location of the predicted ORFs relative to the genomic sequence. This helped to eliminate ORFs that had large overlaps with other ORFs, although there are known cases of ORFs being totally embedded within other ORFs (25, 33).

The initial annotation of these Streptococcus pyogenes ORFs was performed using the BLAST v. 2.0 Gapped search algorithm, BLASTp, to identify homologous sequences. A cutoff “e” value of anything <e⁻¹⁰ was considered significant. Other search algorithms, including FASTA and PSI-BLAST, were also used. The non-redundant protein sequence databases used for the homology searches included GenBank, SWISS-PROT, PIR, and TREMBL database sequences updated daily. ORFs with a BLASTp result of >e⁻¹⁰ were considered to be unique to Streptococcus pyogenes.

Currently, about 60% of all ORFs within a bacterial genome have some match with a protein whose function has been determined. That leaves about 40% of genomic ORFs still uncharacterized. A keyword search of the entire Blast results was carried out using known or suspected candidate target genes as well as words that identified the location of a protein or function. In addition, a keyword search was performed of all MEDLINE references associated with the initial Blast results to look for additional information regarding the ORFs. The keyword search included, for example, the following search terms: adhesin(ion); fibronectin; fibrinogen; collagen; transporter; exporter; extracellular; transferase; surface; and binding. Blast analysis of the ORFs resulted in 1005 ORFS listed as unclassified, 284 ORFs appeared to be specific to Streptococcus pyogenes since they produced Blast similarity only with proteins from this organism, and 676 ORFs were associated with a Medline reference.

For DNA analysis, the % G+C content within each gene was identified. The % G+C content of an ORF was calculated as the (G+C) content of the third nucleotide position of all the codons within an ORF. The value reported was the difference of this value from the arithmetic mean of such values obtained for all ORFs found in the organism. An absolute value ≧8 was considered important for further analysis, as these ORFs may have arisen from horizontal transfer as has been shown in the case of cag pathogenicity island from H. pylori (2), a pattern in keeping with many other pathogenicity islands (22). ORFs that were significantly different in their G+C content totaled 289. These ORFs were further examined for similarity to virulence factors acquired from another organism by horizontal transfer.

Several parameters were used to determine partitioning of the predicted proteins. Proteins destined for translocation across the cytoplasmic membrane encode a leader signal (also known as a signal sequence) composed of a central hydrophobic region flanked at the N-terminus by positively charged residues (56). The program SignalP was used to identify signal peptides and their cleavage sites (46). During expression, the signal peptide is cleaved to produce a mature peptide. In addition, to predict protein localization in bacteria, the software PSORT was used (44). PSORT uses a neural net algorithm to predict localization of proteins to the cytoplasm, periplasm, and/or cytoplasmic membrane for Gram-positive bacteria as well as outer membrane for Gram-negative bacteria. PSORT identified 40 ORFs predicted to be surface exposed (Table V).

TABLE V
Open Reading Frames (ORFs) encoding
putative extracellular proteins

| | | | | | | |
|---|---|---|---|---|---|---|
| 68 | 705 | 1202 | 1664 | 1723 | 2020 | 2385 |
| 165 | 729 | 1310 | 1667 | 1777 | 2046 | 2414 |
| 252 | 788 | 1358 | 1678 | 1909 | 2170 | 2437 |
| 510 | 1058 | 1362 | 1680 | 1972 | 2236 | 2601 |
| 601 | 1132 | 1573 | 1681 | 1975 | 2250 | |
| 668 | 1200 | 1638 | 1683 | 2014 | 2300 | |

In addition, transmembrane (TM) domains of proteins were analyzed using the software program TopPred2 (10). This program predicts regions of a protein that are hydrophobic that may potentially span the lipid bilayer of the membrane. Analysis by TopPred2 for hydrophobic regions of a protein that may potentially span the lipid bilayer of the membrane identified 48 ORFs that encoded putative proteins with three or more transmembrane spanning domains (Table VI) and are thus considered to be membrane bound.

TABLE VI
Open Reading Frames (ORFs) encoding putative proteins
with three or greater transmembrane regions

| | | | | | | |
|---|---|---|---|---|---|---|
| 8 | 307 | 594 | 752 | 1222 | 1598 | 2069 |
| 73 | 312 | 613 | 844 | 1266 | 1657 | 2091 |
| 80 | 395 | 650 | 925 | 1317 | 1708 | 2227 |
| 95 | 508 | 672 | 975 | 1488 | 1726 | 2283 |
| 141 | 551 | 706 | 1018 | 1496 | 1779 | 2424 |
| 265 | 567 | 708 | 1152 | 1513 | 1999 | 2562 |
| 306 | 593 | 731 | 1156 | 1596 | 2002 | |

The Hidden Markov Model (HMM) Pfam database of multiple alignments of protein domains or conserved protein regions (61) was used to identify Streptococcus pyogenes proteins that may belong to an existing protein family. Keyword searching of this output was used to identify proteins that might have been missed by the Blast search criteria. HMM models were also developed by inventor's assignee. A computer algorithm, HMM Lipo, was developed to predict lipoproteins using 132 biologically characterized non-Streptococcus pyogenes bacterial lipoproteins from over 30 organisms. This training set was generated from experimentally proven prokaryotic lipoproteins. HMM Lipo identified 30 ORFs that are putative lipoproteins (Table VII).

TABLE VII
Open Reading Frames (ORFs) encoding putative lipoproteins

| | | | | | |
|---|---|---|---|---|---|
| 68 | 601 | 747 | 1659 | 1789 | 1983 |
| 309 | 678 | 1157 | 1664 | 1818 | 2417 |
| 347 | 685 | 1202 | 1723 | 1878 | 2452 |
| 540 | 704 | 1284 | 1755 | 1882 | 2459 |
| 554 | 729 | 1495 | 1788 | 1918 | 2601 |

In addition, 15 ORFs were predicted to have a LPXTG motif and were classified as proteins that might be targeted by sortase (Table VIII).

TABLE VIII
Open Reading Frames (ORFs) encoding putative
proteins containing the LPXTG motif

| | | | |
|---|---|---|---|
| 433 | 1218 | 1854 | 2450 |
| 608 | 1316 | 2019 | 2477 |
| 967 | 1330 | 2434 | 2497 |
| 1191 | 1698 | 2446 | |

SEQ ID NOS: 669-674 contain the nucleotide and amino acid sequences of the proteins Grab (ORF 608), M protein (ORF 2434), and ScpA (ORF 2446), respectively.

Furthermore, using about 70 known prokaryotic proteins containing the LPXTG cell wall sorting signal, a HMM (15) was developed to predict cell wall proteins that are anchored to the peptidoglycan layer (38, 45). The model used not only the LPXTG sequence, but also included two features of the downstream sequence, the hydrophobic transmembrane domain and the positively charged carboxy terminus. There were 5 proteins identified as potentially binding to the peptidoglycan layer in a non-covalent manner independently of the sortase (Table IX).

TABLE IX
Open Reading Frames (ORFs) encoding putative
peptidoglycan binding proteins

| | | | | |
|---|---|---|---|---|
| 898 | 1569 | 1675 | 2266 | 2311 |

The proteins encoded by the identified ORFs were also evaluated for other characteristics. A tandem repeat finder (5) identified ORFs containing repeated DNA sequences such as those found in MSCRAMMs (20) and phase variable surface proteins of Neisseria meningitidis (51). There were 23 ORFs found to encode proteins containing such repeat regions (Table X).

TABLE X
Open Reading Frames (ORFs) encoding putative
proteins containing repeat regions

| | | | | | |
|---|---|---|---|---|---|
| 218 | 433 | 1149 | 1783 | 2422 | 2513 |
| 265 | 555 | 1562 | 1972 | 2434 | 2590 |
| 336 | 699 | 1583 | 2137 | 2437 | 2618 |
| 431 | 783 | 1683 | 2231 | 2477 | |

In addition, proteins that contain the Arg-Gly-Asp (RGD) attachment motif, together with integrins that serve as their receptor, constitute a major recognition system for cell adhesion. RGD recognition is one mechanism used by microbes to gain entry into eukaryotic tissues (29, 63). There were 65 ORFs identified that encoded RGD-containing proteins (Table XI).

TABLE XI
Open Reading Frames (ORFs) encoding putative
proteins containing the RGD motif.

| | | | | | | |
|---|---|---|---|---|---|---|
| 18 | 544 | 885 | 1149 | 1504 | 1957 | 2379 |
| 201 | 626 | 889 | 1161 | 1626 | 2042 | 2414 |
| 209 | 641 | 899 | 1200 | 1643 | 2054 | 2446 |
| 302 | 654 | 967 | 1274 | 1657 | 2082 | 2558 |
| 344 | 667 | 968 | 1313 | 1675 | 2148 | 2570 |
| 350 | 668 | 1010 | 1316 | 1773 | 2205 | |
| 396 | 695 | 1027 | 1373 | 1779 | 2247 | |
| 397 | 726 | 1074 | 1401 | 1885 | 2253 | |
| 413 | 787 | 1108 | 1416 | 1891 | 2287 | |
| 526 | 829 | 1110 | 1431 | 1901 | 2335 | |

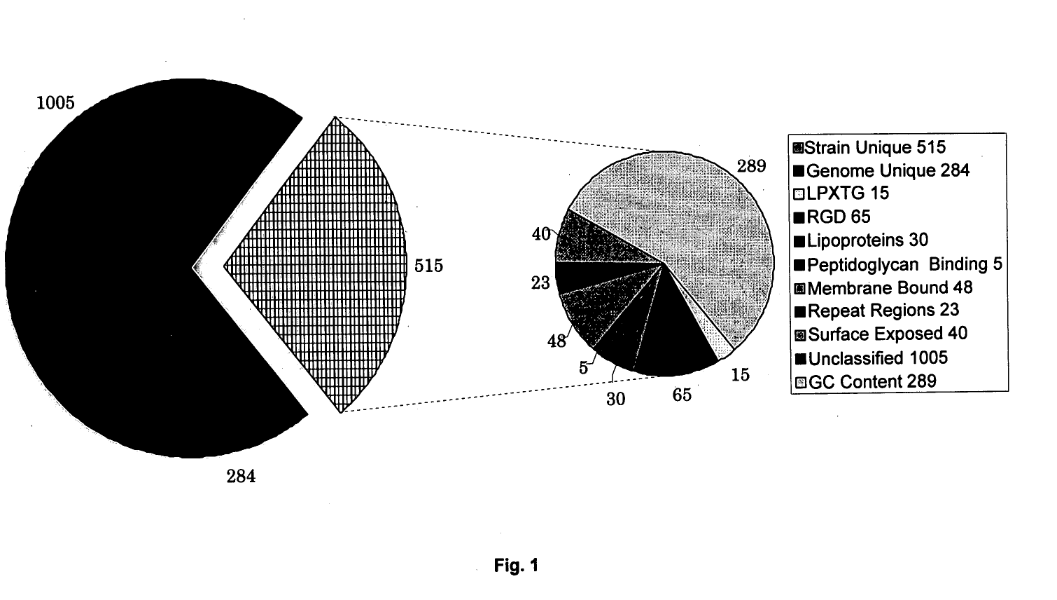

A graphical representation of the results of the genomic analysis and ORF identification is depicted in FIG. 1.

Proteomic Approach

As stated above, a proteomic approach was also taken to identify surface localized proteins of Streptococcus pyogenes.

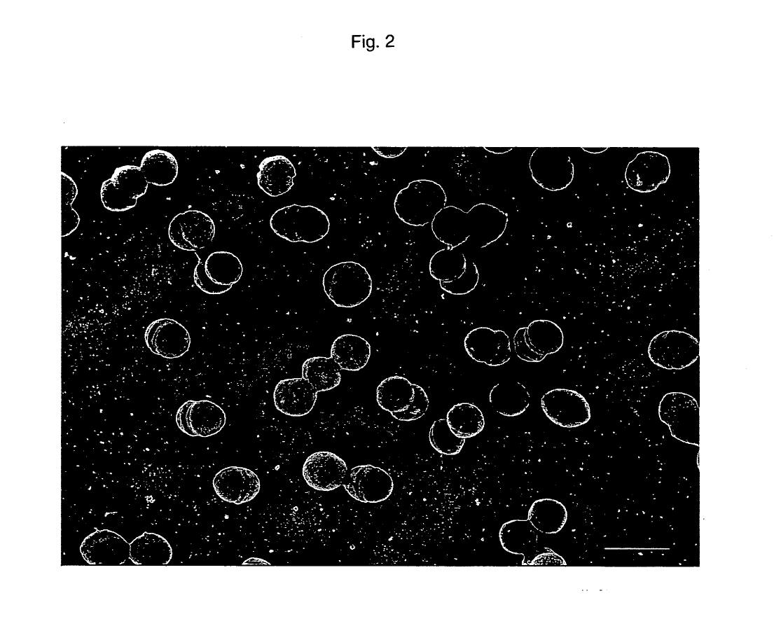

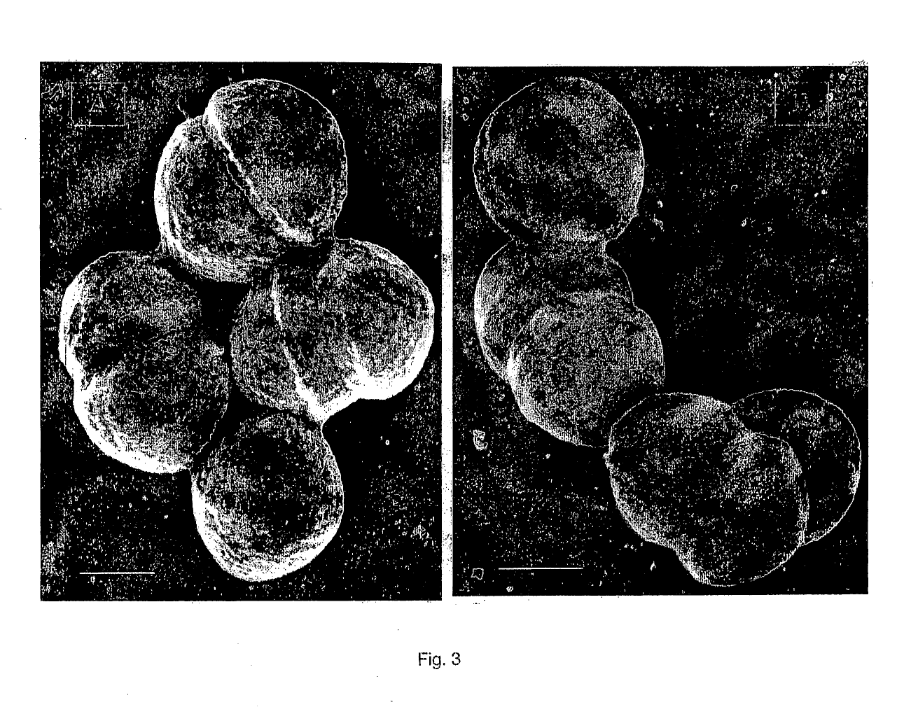

In order to identify only those proteins localized to the surface of the cell, care was taken during the preparation and digestion of the Streptococcus pyogenes cells with trypsin. Samples of the cells were taken just prior to the addition of trypsin and at the completion of the digestion, and were examined for cell integrity by viable counts and LV-SEM. Following digestion, untreated cells clearly aggregated and adhered to the side of the tube, while the treated cells formed an even cell suspension. Viable counts showed no significant difference between samples and in fact were slightly higher in the treated cells due to the aggregation of the untreated sample. LV-SEM confirmed these results (FIG. 2). Digested cells were evenly and individually distributed over the cover slip, while the untreated sample displayed large clumps of bacteria. Topographical examination at high magnification of untreated bacterial cells displayed large quantities of surface material typical of Streptococcus pyogenes. However, individual cells in the trypsin digested sample showed the reduction of all observable surface protein as the cells appeared bald and devoid of any surface material. FIG. 3 depicts LV-SEMS of Streptococcus pyogenes before (left panel, Panel A) and after (right panel, Panel B) digestion with trypsin. The cells before digestion with trypsin (Panel A) are larger and display surface material. The LV-SEM of the cells after digestion (Panel B) are smaller and appear devoid of any surface protein.

In order to identify the peptide components of the complex surface digest mixture, an analytical technique was used to separate and sequence multiple peptides with high sensitivity over a large concentration range. Tandem mass spectrometry (MS/MS) has been shown to be a powerful approach to analyze proteins from both gels and in solution (17). MS/MS first uses a mass analyzer to separate a peptide ion from a mixture of ions, then uses a second step or mass analyzer to activate and dissociate the ion of interest. This process, known as collision induced dissociation (CID), causes the peptide to fragment at the peptide bonds between the amino acids, and therefore, the fragmentation pattern of a peptide is used to determine its amino acid sequence.

In addition, the SEQUEST computer algorithm was used to search the experimental fragmentation spectrum directly against protein or translated nucleotide sequence databases. For peptides above roughly 800-900 Da in size, a single spectrum can uniquely identify a protein.

To sequence multiple peptides from a complex mixture, a reversed phase chromatography system was coupled to an electrospray ion trap mass spectrometer. In this system, it is known that high sensitivity (down to sub-femtomole levels) can be attained by minimizing both flow rate and column diameter to concentrate the elution volume and direct as much of the column effluent as possible into the orifice of the mass spectrometer detector. Initial experiments separated peptides using a reversed phase gradient of 1% acetonitrile/min. In order to increase chromatographic separation, longer gradients, down to 0.28% acetonitrile/min., and slower flow rates (50 nL/min.) were later employed. To maximize the coverage of proteins present in the sample, the data-dependent acquisition feature of the ion trap was employed.

Dynamic exclusion was used to prevent reacquisition of tandem mass spectra of ions once a spectrum had been acquired for a particular n/z value. The isotopic exclusion function excluded the ion associated with the ¹³C isotope of peptides from the list of ions slated for MS/MS. A 3-u mass width window was selected for this purpose. Using these data-dependent features dramatically increased the number of peptide ions that were selected for CID analysis.

The LC-MS/MS data acquisition conditions described above typically resulted in fragmentation data for more than 2000 peptide ions for each run. Using the SEQUEST algorithm, this data was searched against a composite protein sequence database containing the translated ORFs from Streptococcus pyogenes combined with the non-redundant protein sequence database OWL. SEQUEST search conditions used modified trypsin selectivity and allowed a differential search of +16 Da on methionine to account for methionine oxidation. Candidate matches identified by SEQUEST were confirmed using the following manual procedure. Those matches with Xcorr values greater than 2.5 (a measure of the similarity of the experimental ms/ms data to that generated from the sequence database) and delCn values greater than 0.1 (delCn measures the normalized difference between the Xcorr values of the first and second matches) were chosen for further analysis. The fragmentation spectra from good matches were checked for reasonable signal/noise, and the list of matched ions was examined for reasonable continuity. Some matches that were not acceptable alone were included if other confirmatory ms/ms data was generated by the same sample. The ORFs obtained by this proteomic approach are presented in Table XII.

TABLE XII
Open Reading Frames (ORFs) identified by tryptic digestion

| | | | | | | |
|---|---|---|---|---|---|---|
| 66 | 678 | 1224 | 1638 | 1878 | 2214 | 2459 |
| 102 | 704 | 1234 | 1650 | 1902 | 2330 | 2477 |
| 145 | 743 | 1237 | 1654 | 1943 | 2354 | 2586 |
| 232 | 825 | 1238 | 1659 | 1975 | 2377 | 2593 |
| 238 | 850 | 1253 | 1698 | 2019 | 2379 | 2601 |
| 436 | 934 | 1284 | 1788 | 2064 | 2387 | |
| 516 | 993 | 1316 | 1794 | 2086 | 2417 | |
| 554 | 1036 | 1330 | 1816 | 2106 | 2420 | |
| 589 | 1140 | 1358 | 1818 | 2116 | 2422 | |
| 608 | 1157 | 1487 | 1819 | 2120 | 2434 | |
| 661 | 1191 | 1495 | 1850 | 2123 | 2446 | |
| 668 | 1218 | 1557 | 1854 | 2202 | 2450 | |

Several of the ORFs identified were cloned and expressed. Mouse antisera, generated to the purified proteins, were first analyzed for reactivity by ELISA using the same preparation used for the mouse immunization as the coating antigen. To quantitate protein expression on the surface of Streptococcus pyogenes, these sera were then used in whole cell ELISAs. To qualify the protein expression of the specific proteins, whole Streptococcus pyogenes cells were labeled by immunogold and viewed by LV-SEM.

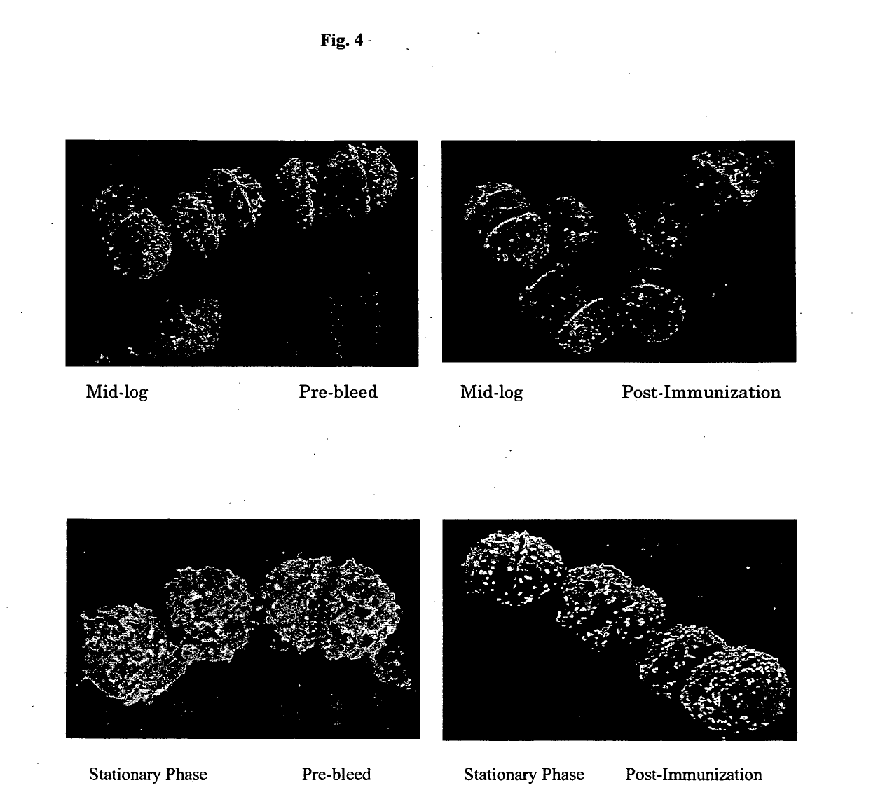

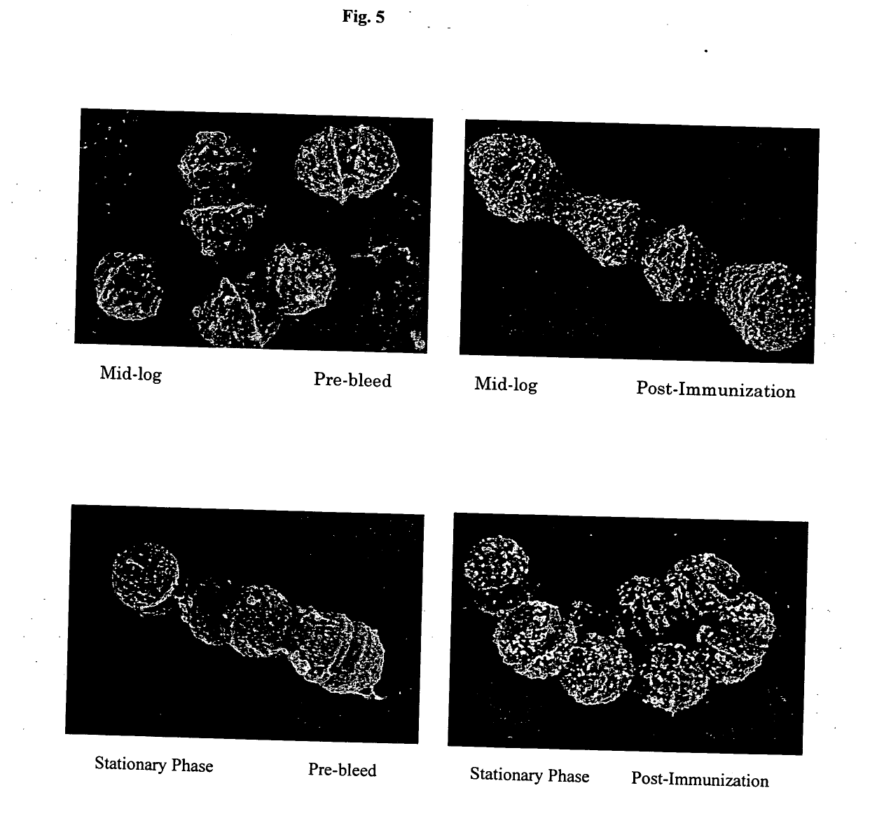

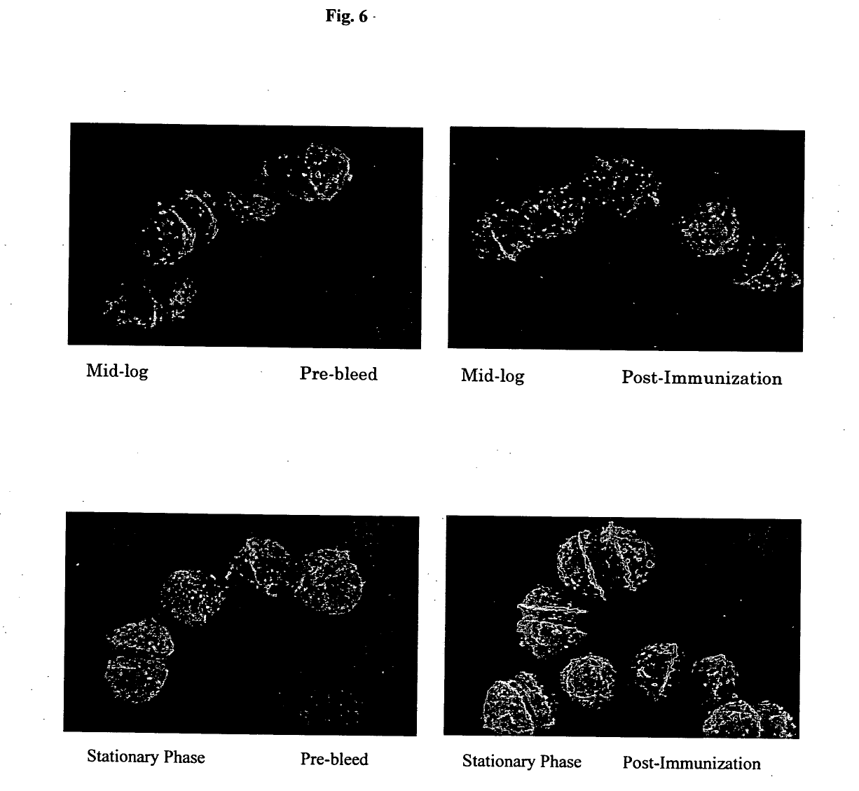

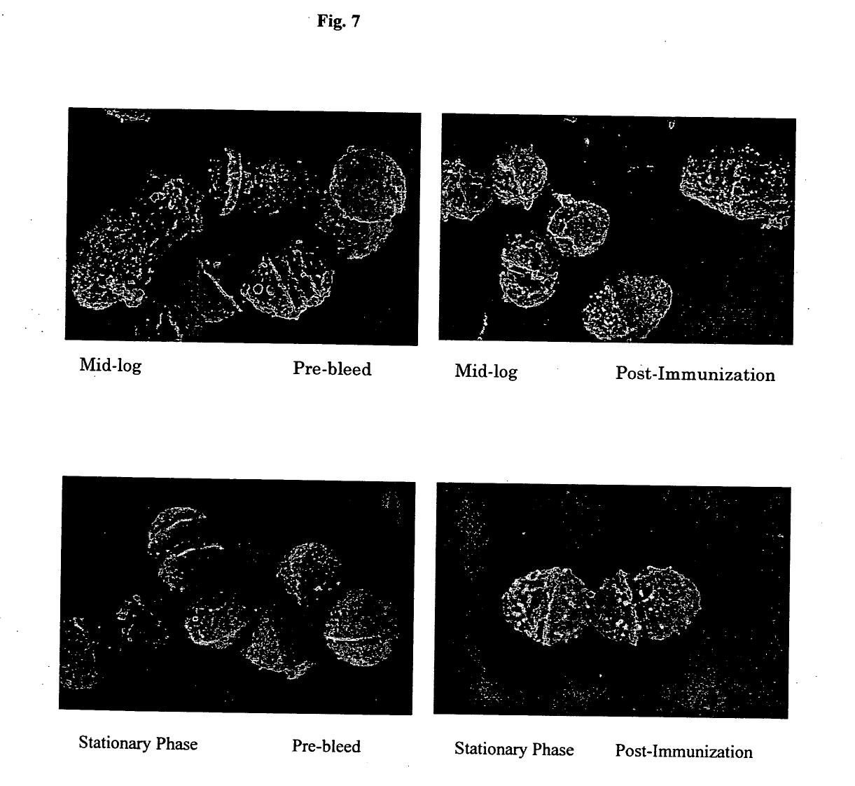

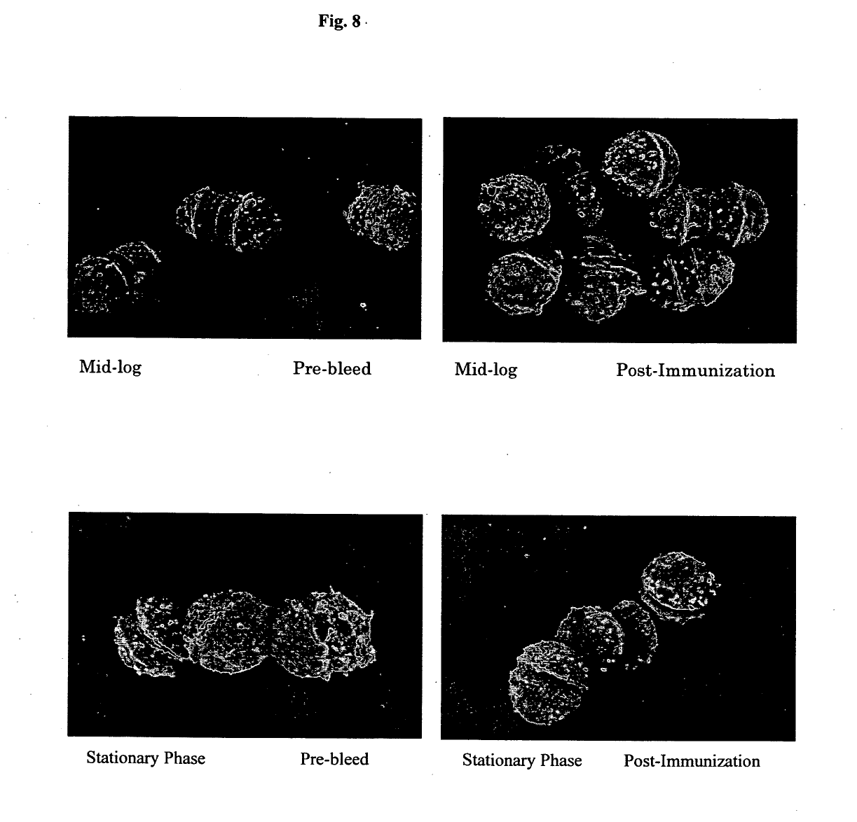

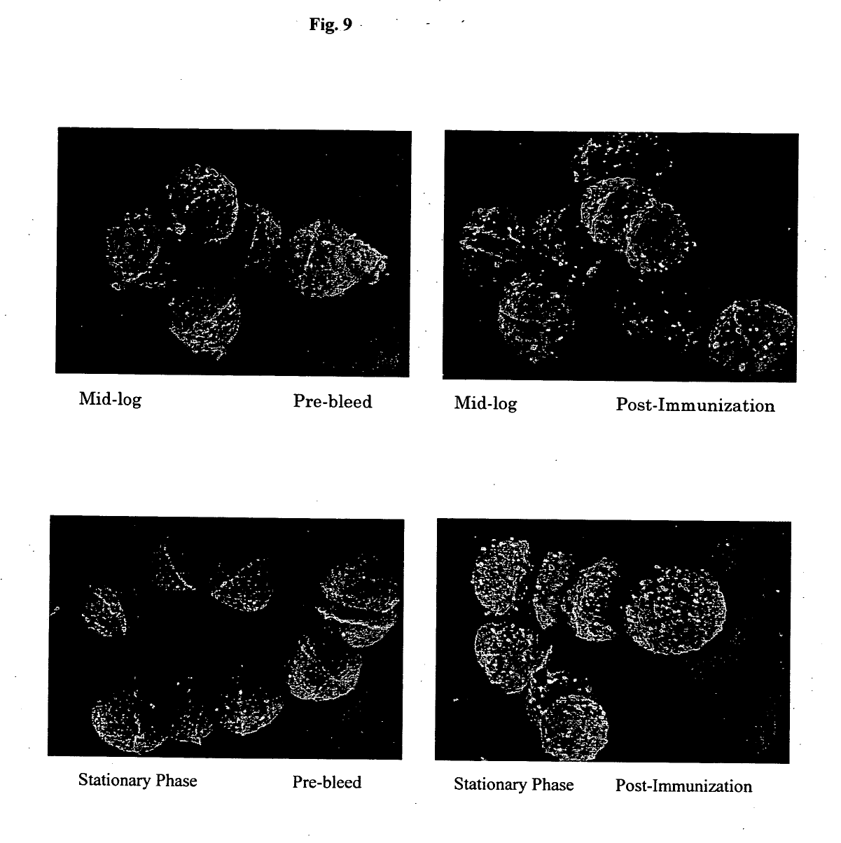

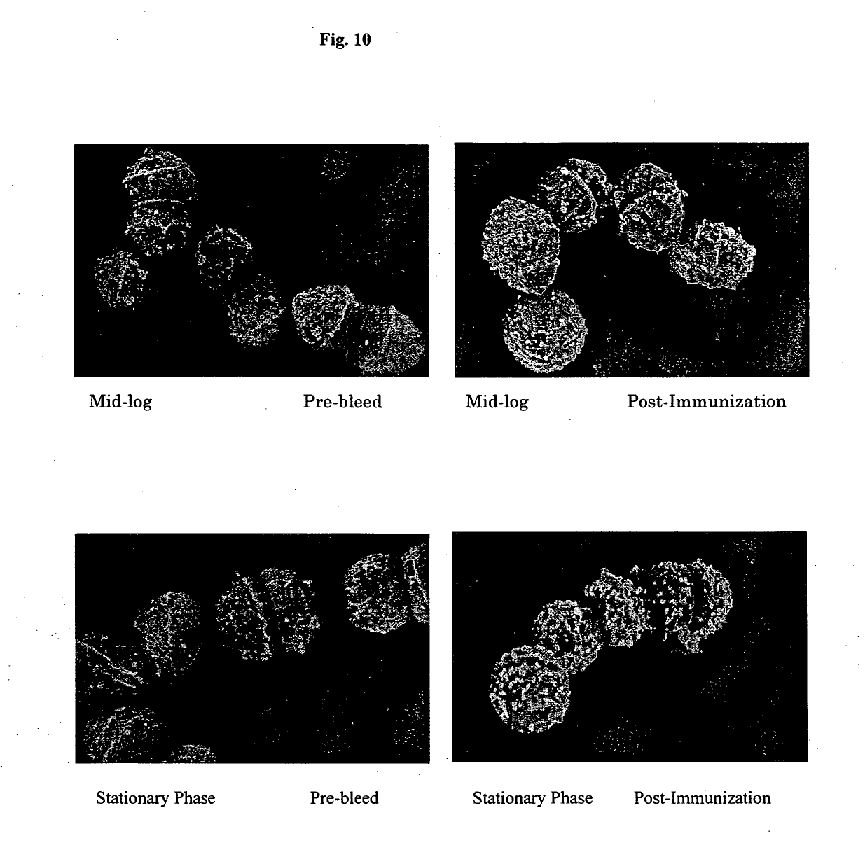

For some of the identified ORFs, the encoded proteins were observed to be expressed in a manner that was dependent upon phase of growth (mid-log versus stationary). Examples of this class are ORF 218 (FIG. 4), ORF 554 (FIG. 5), and ORF 1191 (FIG. 6). In some cases, expression level was higher in the mid-log growth, while others were greater in the stationary cells. Proteins encoded by other ORFs were expressed at low levels regardless of growth phase (ORFs 2064, 2601, and 1316) (shown in FIGS. 7-9, respectively), while others were expressed at high levels independent of growth phase (ORF 1224) (FIG. 10). As a positive control, anti-C5a peptidase sera was used as it is known to be expressed and localized to the cell wall of Streptococcus pyogenes. All antisera showed an increase in reactivity over the respective pre-immune control sera.

Combination of Genomic and Proteomic Approaches

The ORFs identified in Tables V-XII were then categorized into one of four groups: ORFs encoding surface localized proteins identified by proteomics (Table I); ORFs encoding putative lipoproteins (Table II); ORFs encoding putative polypeptides containing a LPXTG motif (Table III); and ORFs encoding other putative surface localized polypeptides (Table IV). Tables I-IV are provided supra. It should be apparent that the ORFs contained in Tables I-IV are non-redundant, i.e., the ORFs listed in Tables I-IV each appear once though many possess characteristics that match another table.

The nucleotide sequences of Table I encode polypeptides that have been identified by the proteomic approach as being surface localized, Streptococcus pyogenes proteins. The nucleotide sequences of Tables II-IV encode putative polypeptides that have been identified by the described genomic approaches as being surface localized, Streptococcus pyogenes proteins. Specifically, the nucleotide sequences of Table II encode putative lipoproteins, the nucleotide sequences of Table III encode putative proteins having an LPXTG cell wall sorting signal, and the nucleotide sequences of Table IV encode putative surface localized proteins that include at least one of several criteria, as described herein, including similarity to other proteins for which a function and cellular location had been previously identified, match with a protein family (e.g., Pfam), and a combined analysis of the membrane spanning domains, Psort and sigP values, and the predicted molecular weight of the protein.

Each of odd numbered SEQ ID NOS: 1-667 encodes an amino acid sequence that is numbered consecutively after the nucleotide sequence. Thus, for example, the nucleotide sequence of SEQ ID NO: 1 encodes the amino acid sequence of SEQ ID NO: 2, and the nucleotide sequence of SEQ ID NO: 3 encodes the amino acid sequence of SEQ ID NO: 4, etc.

Polypeptides

The invention provides Streptococcus pyogenes polypeptides that are surface localized. Specifically, the polypeptides of the invention include isolated polypeptides that comprise an amino acid sequence of any of even numbered SEQ ID NOS: 2-668, i.e., SEQ ID NO: 2, 4, 6, 8, 10, 12, 14, 16, 18, 20, 22, 24, 26, 26, 30, 32, 34, 36, 38, 40, 42, 44, 46, 48, 50, 52, 54, 56, 58, 60, 62, 64, 66, 68, 70, 72; 74, 76, 78, 80, 82, 84, 86, 88, 90, 92, 94, 96, 98, 100, 102, 104, 106, 108, 110, 112, 114, 116, 118, 120; 122, 124, 126, 128, 130, 132, 134, 136; 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490,492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, or 668.

The polypeptides of the invention also include isolated polypeptides that consist essentially of the aforementioned amino acid sequences and isolated polypeptides that consist of the aforementioned amino acid sequences. The term “isolated” means altered by the hand of man from the natural state. If an “isolated” composition or substance occurs in nature, it has been changed or removed from its original environment, or both. For example, a polypeptide or a polynucleotide naturally present in a living animal is not “isolated,” but the same polypeptide of polynucleotide separated from the coexisting materials of its natural state is “isolated”, as the term is employed herein. As used herein, the term “isolated” contemplates a polypeptide (or other component) that is isolated from its natural source and/or prepared using recombinant technology.

A polypeptide sequence of the invention may be identical to the reference sequence of even numbered SEQ ID NOS: 2-668, that is, 100% identical, or it may include up to a certain integer number of amino acid alterations as compared to the reference sequence such that the % identity is less than 100%. Such alterations include at least one amino acid deletion, substitution, including conservative and non-conservative substitution, or insertion. The alterations may occur at the amino- or carboxy-terminal positions of the reference polypeptide sequence or anywhere between those terminal positions, interspersed either individually among the amino acids in the reference amino acid sequence or in one or more contiguous groups within the reference amino acid sequence.

Thus, the invention also provides isolated polypeptides having sequence identity to the amino acid sequences contained in the Sequence Listing (i.e., even numbered SEQ ID NOS: 2-668). Depending on the particular sequence, the degree of sequence identity is preferably greater than 50% (e.g., 60%, 70%, 80%, 90%, 95%, 97%, 99% or more). These homologous proteins include mutants and allelic variants.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al. 1984), BLASTP, BLASTN, and FASTA (Altschul, S. F., et al., 1990. The BLASTX program is publicly available from NCBI and other sources (BLAST Manual, Altschul, S., et al., NCBI NLM NIH Bethesda, Md. 20894; Altschul, S., et al., 1990). The well known Smith Waterman algorithm may also be used to determine identity.

For example, the number of amino acid alterations for a given % identity can be determined by multiplying the total number of amino acids in one of even numbered SEQ ID NOS: 2-668 by the numerical percent of the respective percent identity (divided by 100) and then subtracting that product from said total number of amino acids in the one of even numbered SEQ ID NOS: 2-668, or:
nₐ≦xₐ−(xₐ·y),
wherein nₐ is the number of amino acid alterations, xₐ is the total number of amino acids in the one of SEQ ID NOS: 2-668, and y is, for instance, 0.70 for 70%, 0.80 for 80%, 0.85 for 85% etc., and wherein any non-integer product of xₐ and y is rounded down to the nearest integer prior to subtracting it from xₐ.

The present invention contemplates isolated polypeptides that are substantially conserved across strains of β-hemolytic streptococci. Further, isolated polypeptides that are substantially conserved across strains of β-hemolytic streptococci and that are effective in preventing or ameliorating a β-hemolytic streptococcal colonization or infection in a susceptible subject are also contemplated by the present invention. As used herein, the term “conserved” refers to, for example, the number of amino acids that do not undergo insertions, substitution and/or deletions as a percentage of the total number of amino acids in a protein. For example, if a protein is 55% conserved and has, for example, 263 amino acids, then there are 144 amino acid positions in the protein at which amino acids do not undergo substitution. Likewise, if a protein is 90% conserved and has, for example, about 280 amino acids, then there are 28 amino acid positions at which amino acids may undergo substitution and 252 (i.e., 280 minus 28) amino acid positions at which the amino acids do not undergo substitution. According to an embodiment of the present invention, the isolated polypeptide is preferably at least about 80% conserved across the strains of β-hemolytic streptococci, more preferably at least about 85% conserved across the strains, even more preferably at least about 90% conserved across the strains, and most preferably at least about 95% conserved across the strains, without limitation.

Modifications and changes can be made in the structure of the polypeptides of even numbered SEQ ID NOS: 2-668 and still obtain a molecule having β-hemolytic streptococci and/or Streptococcus pyogenes activity and/or antigenicity. For example, certain amino acids can be substituted for other amino acids in a sequence without appreciable loss of activity and/or antigenicity. Because it is the interactive capacity and nature of a polypeptide that defines that polypeptide's biological functional activity, certain amino acid sequence substitutions can be made in a polypeptide sequence (or, of course, its underlying DNA coding sequence) and nevertheless obtain a polypeptide with like properties.

The invention includes any isolated polypeptide which is a biological equivalent that provides the desired reactivity as described herein. The term “desired reactivity” refers to reactivity that would be recognized by a person skilled in the art as being a useful result for the purposes of the invention. Examples of desired reactivity are described herein, including without limitation, desired levels of protection, desired antibody titers, desired opsonophagocytic activity and/or desired cross-reactivity, such as would be recognized by a person skilled in the art as being useful for the purposes of the present invention. The desired opsonophagocytic activity is indicated by a percent killing of bacteria as measured by decrease in colony forming units (CFU) in OPA versus a negative control. Without being limited thereto, the desired opsonophagocytic activity is preferably at least about 15%, more preferably at least about 20%, even more preferably at least about 40%, even more preferably at least about 50% and most preferably at least about 60%.

The invention includes polypeptides that are variants of the polypeptides comprising an amino acid sequence of SEQ ID NOS: 2-668. “Variant” as the term is used herein, includes a polypeptide that differs from a reference polypeptide, but retains essential properties. Generally, differences are limited so that the sequences of the reference polypeptide and the variant are closely similar overall and, in many regions, identical (i.e., biologically equivalent). A variant and reference polypeptide may differ in amino acid sequence by one or more substitutions, additions, or deletions in any combination. A substituted or inserted amino acid residue may or may not be one encoded by the genetic code. A variant of a polypeptide may be a naturally occurring such as an allelic variant, or it may be a variant that is not known to occur naturally. Non-naturally occurring variants of polypeptides may be made by direct synthesis or by mutagenesis techniques.

In making such changes, the hydropathic index of amino acids can be considered. The importance of the hydropathic amino acid index in conferring interactive biologic function on a polypeptide is generally understood in the art (Kyte & Doolittle, 1982). It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are listed in parentheses after each amino acid as follows: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid residue determines the secondary and tertiary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within +/−2 is preferred, those which are within +/−1 are particularly preferred, and those within +/−0.5 are even more particularly preferred.

Substitution of like amino acids can also be made on the basis of hydrophilicity, particularly where the biological functional equivalent polypeptide or peptide thereby created is intended for use in immunological embodiments. U.S. Pat. No. 4,554,101, incorporated herein by reference, states that the greatest local average hydrophilicity of a polypeptide, as governed by the hydrophilicity of its adjacent amino acids, correlates with its immunogenicity and antigenicity, i.e., with a biological property of the polypeptide.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). It is understood that an amino acid can be substituted for another having a similar hydrophilicity value and still obtain a biologically equivalent and in particular, an immunologically equivalent, polypeptide. In such changes, the substitution of amino acids whose hydrophilicity values are within ±2 is preferred, those which are within ±1 are particularly preferred, and those within ±0.5 are even more particularly preferred.

As outlined above, amino acid substitutions are generally, therefore, based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine, and isoleucine. As shown in Table XIII below, suitable amino acid substitutions include the following:

TABLE XIII

| Original | Exemplary Residue |
|---|---|
| Residue | Substitution |
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Met; Leu; Tyr |
| Ser | Thr |
| Thr | Ser |
| Trp | Tyr |
| Tyr | Trp; Phe |
| Val | Ile; Leu |

Thus, the invention includes functional or biological equivalents of the polypeptides of SEQ ID NOS: 2-668 that contain one or more amino acid substitutions.

Biological or functional equivalents of a polypeptide can also be prepared using site-specific mutagenesis. Site-specific mutagenesis is a technique useful in the preparation of second generation polypeptides, or biologically, functionally equivalent polypeptides, derived from the sequences thereof, through specific mutagenesis of the underlying DNA. As noted above, such changes can be desirable where amino acid substitutions are desirable. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, the technique of site-specific mutagenesis is well known in the art. As will be appreciated, the technique typically employs a phage vector which can exist in both a single-stranded and double-stranded form. Typically, site-directed mutagenesis in accordance herewith is performed by first obtaining a single-stranded vector which includes within its sequence a DNA sequence which encodes all or a portion of the Streptococcus pyogenes polypeptide sequence selected. An oligonucleotide primer bearing the desired mutated sequence is prepared, for example, by well known techniques (e.g., synthetically). This primer is then annealed to the single-stranded vector, and extended by the use of enzymes, such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutation. Commercially available kits provide the necessary reagents.

The polypeptides and polypeptide antigens of the invention are understood to include any polypeptide comprising substantial sequence similarity, structural similarity, and/or functional similarity to a polypeptide comprising an amino acid sequence of any of SEQ ID NOS: 2-668. In addition, a polypeptide or polypeptide antigen of the invention is not limited to a particular source. Thus, the invention provides for the general detection and isolation of the polypeptides from a variety of sources.

The polypeptides of the invention may advantageously be cleaved into fragments for use in further structural or functional analysis, or in the generation of reagents such as Streptococcus pyogenes-related polypeptides and Streptococcus pyogenes-specific antibodies. This can be accomplished by treating purified or unpurified polypeptides of the invention with a peptidase such as endoproteinase glu-C (Boehringer, Indianapolis, Ind.). Treatment with CNBr is another method by which peptide fragments may be produced from natural Streptococcus pyogenes polypeptides. Recombinant techniques also can be used to produce specific fragments of a Streptococcus pyogenes polypeptide.

In addition, the inventors contemplate that compounds sterically similar to a particular Streptococcus pyogenes polypeptide antigen may be formulated to mimic the key portions of the peptide structure, known in the art as peptidomimetics. Mimetics are peptide-containing molecules which mimic elements of protein secondary structure. The underlying rationale behind the use of peptidomimetics is that the peptide backbone of proteins exists chiefly to orient amino acid side chains in such a way as to facilitate molecular interactions, such as those of receptor and ligand.

The invention also includes fusion proteins comprising at least one polypeptide of the invention. “Fusion protein” refers to a protein encoded by two, often unrelated, fused genes or fragments thereof. For example, fusion proteins comprising various portions of constant region of immunoglobulin molecules together with another human protein or part thereof have been described. In many cases, employing an immunoglobulin Fc region as a part of a fusion protein is advantageous for use in therapy and diagnosis resulting in, for example, improved pharmacokinetic properties (See, for example, EP-A 0232 2621). On the other hand, for some uses it would be desirable to be able to delete the Fc part after the fusion protein has been expressed, detected, and purified.

The polypeptides of the invention may be in the form of the “mature” protein or may be a part of a larger protein such as a fusion protein. It is often advantageous to include an additional amino acid sequence which contains, for example, secretory or leader sequences, pro-sequences, sequences which aid in purification such as multiple histidine residues, or an additional sequence for stability during recombinant production.

Fragments of the Streptococcus pyogenes polypeptides are also included in the invention. A fragment is a polypeptide having an amino acid sequence that entirely is the same as part, but not all, of the amino acid sequence. The fragment can comprise, for example, at least 7 or more (e.g., 8, 10, 12, 14, 16, 18, 20, or more) contiguous amino acids of an amino acid sequence of any of even numbered SEQ ID NOS: 2-668. Fragments may be “freestanding” or comprised within a larger polypeptide of which they form a part or region, most preferably as a single, continuous region. In one embodiment, the fragments include at least one epitope of the mature polypeptide sequence.

The polypeptides of the invention can be prepared in any suitable manner. Such polypeptides include naturally occurring polypeptides, recombinantly produced polypeptides, synthetically produced polypeptides, and polypeptides produced by a combination of these methods. Means for preparing such polypeptides are well understood in the art.

Polynucleotides

The invention also provides isolated polynucleotides comprising a nucleotide sequence that encodes a polypeptide of the invention, and polynucleotides closely related thereto. These polynucleotides include:

(i) an isolated polynucleotide comprising a nucleotide sequence of any of odd numbered SEQ ID NOS: 1-147 (Table I);

(ii) an isolated polynucleotide comprising a nucleotide sequence of any of odd numbered SEQ ID NOS: 149-181 (Table II);

(iii) an isolated polynucleotide comprising a nucleotide sequence of any of odd numbered SEQ ID NOS: 183-187 (Table III); and

(iv) an isolated polynucleotide comprising a nucleotide sequence of any of odd numbered SEQ ID NOS: 189-667 (Table IV).

The polynucleotides encoding the polypeptides of the invention may be identical to the nucleotide sequences contained in Tables I-IV or they may have variant sequences which, as a result of the redundancy (degeneracy) of the genetic code, also encode polypeptides of the invention.

Further, the invention provides isolated polynucleotides having sequence identity to the nucleotide sequences of SEQ ID NOS: 1-667. Depending on the particular sequence, the degree of sequence identity is preferably greater than 70% (e.g., 80%, 90%, 95%, 97% 99% or more).

As discussed above, “identity,” as known in the art, is a relationship between two or more polypeptide sequences or two or more polynucleotide sequences, as determined by comparing the sequences. “Identity” can be readily calculated by known methods. By way of example, a polynucleotide sequence of the present invention may be identical to a reference nucleotide sequence of odd numbered SEQ ID NOS: 1-667, that is be 100% identical, or it may include up to a certain integer number of nucleotide alterations as compared to the reference nucleotide sequence. Such alterations include at least one nucleotide deletion, substitution, including transition and transversion, or insertion. The alterations may occur at the 5′ or 3′ terminal positions of the reference nucleotide sequence or anywhere between those terminal positions, interspersed either individually among the nucleotides in the reference sequence or in one or more contiguous groups within the reference nucleotide sequence. The number of nucleotide alterations is determined by multiplying the total number of nucleotides in one of odd numbered SEQ ID NOS: 1-667 by the numerical percent of the respective percent identity (divided by 100) and subtracting that product from said total number of nucleotides of the reference nucleotide sequence of any of odd numbered SEQ ID NOS: 1-667.

For example, for a polynucleotide that has at least 70% identity to a nucleotide sequence of one of odd numbered SEQ ID NOS: 1-667, the polynucleotide may include up to nₙ nucleic acid alterations over the entire length of the nucleotide sequence of one of odd numbered SEQ ID NOS: 1-667, wherein nₙ is calculated by the formula:
nₙ≦xₙ−(xₙ·y),
and wherein xₙ is the total number of nucleotides of the nucleotide sequence of one of odd numbered SEQ ID NOS: 1-667, y has a value of 0.70, and wherein any non-integer product of xₙ and y is rounded down to the nearest integer prior to subtracting such product from xₙ. Of course, y may also have a value of 0.80 for 80%, 0.85 for 85%, 0.90 for 90%, 0.95 for 95%, etc.

The invention also includes polynucleotides that encode polypeptide variants of the polypeptides comprising an amino acid sequence of SEQ ID NOS: 2-668, in which one or more amino acid residues are substituted, deleted, or added, in any combination while retaining the biological activity of the native polypeptide. “Variant” as the term is used herein, is a polynucleotide that differs from a reference polynucleotide, but retains essential properties. Changes in the nucleotide sequence of the variant may or may not alter the amino acid sequence of a polypeptide encoded by the reference polynucleotide. Nucleotide changes may result in amino acid substitutions, additions, deletions, fusions, and truncations in the polypeptide encoded by the reference sequence. A variant of a polynucleotide may be naturally occurring such as an allelic variant, or it may be a variant that is not known to occur naturally. Non-naturally occurring variants of polynucleotides may be made by mutagenesis techniques or by direct synthesis.

The invention also includes polynucleotides capable of hybridizing under reduced stringency conditions, more preferably stringent conditions, and most preferably highly stringent conditions, to polynucleotides described herein. Examples of stringency conditions are shown in the Stringency Conditions Table below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE XIV
STRINGENCY CONDITIONS TABLE

| Stringency | Polynucleotide | Hybrid Length | Hybridization Temperature | Wash Temperature |
|---|---|---|---|---|
| Condition | Hybrid | (bp)¹ | and Buffer^{H} | and Buffer^{H} |
| A | DNA:DNA | >50 | 65° C.; 1 × SSC -or- | 65° C.; 0.3 × SSC |
| | | | 42° C.; 1 × SSC, 50% formamide | |
| B | DNA:DNA | <50 | T_{B}; 1 × SSC | T_{B}; 1 × SSC |
| C | DNA:RNA | >50 | 67° C.; 1 × SSC -or- | 67° C.; 0.3 × SSC |
| | | | 45° C.; 1 × SSC, 50% formamide | |
| D | DNA:RNA | <50 | T_{D}; 1 × SSC | T_{D}; 1 × SSC |
| E | RNA:RNA | >50 | 70° C.; 1 × SSC -or- | 70° C.; 0.3 × SSC |
| | | | 50° C.; 1 × SSC, 50% formamide | |
| F | RNA:RNA | <50 | T_{F}; 1 × SSC | T_{f}; 1 × SSC |
| G | DNA:DNA | >50 | 65° C.; 4 × SSC -or- | 65° C.; 1 × SSC |
| | | | 42° C.; 4 × SSC, 50% formamide | |
| H | DNA:DNA | <50 | T_{H}; 4 × SSC | T_{H}; 4 × SSC |
| I | DNA:RNA | >50 | 67° C.; 4 × SSC -or- | 67° C.; 1 × SSC |
| | | | 45° C.; 4 × SSC, 50% formamide | |
| J | DNA:RNA | <50 | T_{J}; 4 × SSC | T_{J}; 4 × SSC |
| K | RNA:RNA | >50 | 70° C.; 4 × SSC -or- | 67° C.; 1 × SSC |
| | | | 50° C.; 4 × SSC, 50% formamide | |
| L | RNA:RNA | <50 | T_{L}; 2 × SSC | T_{L}; 2 × SSC |
| M | DNA:DNA | >50 | 50° C.; 4 × SSC -or- | 50° C.; 2 × SSC |
| | | | 40° C.; 6 × SSC, 50% formamide | |
| N | DNA:DNA | <50 | T_{N}; 6 × SSC | T_{N}; 6 × SSC |
| O | DNA:RNA | >50 | 55° C.; 4 × SSC -or- | 55° C.; 2 × SSC |
| | | | 42° C.; 6 × SSC, 50% formamide | |
| P | DNA:RNA | <50 | T_{P}; 6 × SSC | T_{P}; 6 × SSC |
| Q | RNA:RNA | >50 | 60° C.; 4 × SSC -or- | 60° C.; 2 × SSC |
| | | | 45° C.; 6 × SSC, 50% formamide | |
| R | RNA:RNA | <50 | T_{R}; 4 × SSC | T_{R}; 4 × SSC |
| bp¹: The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| buffer^{H}: SSPE (1 × SSPE is 0.15 M NaCl, 10 mM NaH₂PO₄, and 1.25 mM EDTA, pH 7.4) can be substituted for SSC (1 × SSC is 0.15 M NaCl and 15 mM sodium citrate) in the hybridization and wash buffers; washes are performed for 15 minutes after hybridization is complete. | | | | |
| T_{B} through T_{R}: The hybridization temperature for hybrids anticipated to be less than 50 base pairs in length should be 5-10EC less than the melting temperature (Tₘ) of the hybrid, where Tₘ is determined according to the following equations. For hybrids less than 18 base pairs in length, Tₘ(EC) = 2(# of A + T | | | | |
| # bases) + 4(# of G + C bases). For hybrids between 18 and 49 base pairs in length, Tₘ(EC) = 81.5 + 16.6(log₁₀[Na⁺]) + 0.41(% G + C) − (600/N), where N is the number of bases in the hybrid, and [Na⁺] is the concentration of sodium ions in the hybridization buffer ([Na⁺] for 1 × SSC = 0.165 M). | | | | |

Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

The invention also provides polynucleotides that are fully complementary to these polynucleotides and also provides antisense sequences. The antisense sequences of the invention, also referred to as antisense oligonucleotides, include both internally generated and externally administered sequences that block expression of polynucleotides encoding the polypeptides of the invention. The antisense sequences of the invention comprise, for example, about 15-20 base pairs. The antisense sequences can be designed, for example, to inhibit transcription by preventing promoter binding to an upstream nontranslated sequence or by preventing translation of a transcript encoding a polypeptide of the invention by preventing the ribosome from binding.

The polynucleotides of the invention are prepared in many ways (e.g., by chemical synthesis, from DNA libraries, from the organism itself) and can take various forms (e.g., single-stranded, double-stranded, vectors, probes, primers). The term “polynucleotide” includes DNA and RNA, and also their analogs, such as those containing modified backbones.

When the polynucleotides of the invention are used for the recombinant production of polypeptides, the polynucleotide may include the coding sequence of the mature polypeptide or a fragment thereof, by itself, the coding sequence of the mature polypeptide or fragment in reading frame with other coding sequences, such as those encoding a leader or secretory sequence, a pre-, pro-, or prepro-protein sequence, or other fusion protein portions. For example, a marker sequence which facilitates purification of the fused polypeptide can be linked to the coding sequence. The polynucleotide may also contain non-coding 5′ and 3′ sequences, such as transcribed, non-translated sequences, splicing and polyadenylation signals, ribosome binding sites, and sequences that stabilize mRNA.

Expression Systems and Vectors

For recombinant production, host cells are genetically engineered to incorporate expression systems, portions thereof, or polynucleotides of the invention. Introduction of polynucleotides into host cells are effected, for example, by methods described in many standard laboratory manuals, such as Davis et al., BASIC METHODS IN MOLECULAR BIOLOGY (1986) and Sambrook et al., MOLECULAR CLONING: A LABORATORY MANUAL, 2nd ed., Cold Spring Harbor Laboratory Press, Cold Spring Harbor, N.Y. (1989), such as calcium phosphate transfection, DEAE-dextran mediated transfection, transvection, microinjection, ultrasound, cationic lipid-mediated transfection, electroporation, transduction, scrape loading, ballistic introduction, or infection.

Representative examples of suitable hosts include bacterial cells (e.g., streptococci, staphylococci, E. coli, Streptomyces and Bacillus subtilis cells), yeast cells (e.g., Pichia, Saccharomyces), mammalian cells (e.g., vero, Chinese hamster ovary, chick embryo fibroblasts, BHK cells, human SW13 cells), and insect cells (e.g., Sf9, Sf21).

The recombinantly produced polypeptides are recovered and purified from recombinant cell cultures by well-known methods, including high performance liquid chromatography, ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography.

A great variety of expression systems are used. Such systems include, among others, chromosomal, episomal and virus-derived systems, e.g., vectors derived from bacterial plasmids, attenuated bacteria such as Salmonella (U.S. Pat. No. 4,837,151) from bacteriophage, from transposons, from yeast episomes, from insertion elements, from yeast chromosomal elements, from viruses such as vaccinia and other poxviruses, sindbis, adenovirus, baculoviruses, papova viruses, such as SV40, fowl pox viruses, pseudorabies viruses and retroviruses, alphaviruses such as Venezuelan equine encephalitis virus (U.S. Pat. No. 5,643,576), nonsegmented negative-stranded RNA viruses such as vesicular stomatitis virus (U.S. Pat. No. 6,168,943), and vectors derived from combinations thereof, such as those derived from plasmid and bacteriophage genetic elements, such as cosmids and phagemids. The expression systems should include control regions that regulate as well as engender expression, such as promoters and other regulatory elements (such as a polyadenylation signal). Generally, any system or vector suitable to maintain, propagate or express polynucleotides to produce a polypeptide in a host may be used. The appropriate nucleotide sequence may be inserted into an expression system by any of a variety of well-known and routine techniques, such as, for example, those set forth in Sambrook et al., MOLECULAR CLONING, A LABORATORY MANUAL (supra).

The invention also provides vectors (e.g., expression vectors, sequencing vectors, cloning vectors) which comprise a polynucleotide or polynucleotides of the invention, host cells which are genetically engineered with vectors of the invention, and production of polypeptides of the invention by recombinant techniques. Cell-free translation systems can also be employed to produce such proteins using RNAs derived from the DNA constructs of the invention.

Preferred vectors are viral vectors, such as lentiviruses, retroviruses, herpes viruses, adenoviruses, adeno-associated viruses, vaccinia virus, baculovirus, and other recombinant viruses with desirable cellular tropism. Thus, a gene encoding a functional or mutant protein or polypeptide, or fragment thereof can be introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both. Targeted gene delivery is described in PCT Publication Number WO 95/28494.

Viral vectors commonly used for in vivo or ex vivo targeting and therapy procedures are DNA-based vectors and retroviral vectors. Methods for constructing and using viral vectors are known in the art (e.g., Miller and Rosman, BioTechniques, 1992, 7:980-990). Preferably, the viral vectors are replication-defective, that is, they are unable to replicate autonomously in the target cell. Preferably, the replication defective virus is a minimal virus, i.e., it retains only the sequences of its genome which are necessary for encapsulating the genome to produce viral particles.

DNA viral vectors include an attenuated or defective DNA virus, such as, but not limited to, herpes simplex virus (HSV), papillomavirus, Epstein Barr virus (EBV), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt et al., Molec. Cell. Neurosci., 1991, 2:320-330), defective herpes virus vector lacking a glycoprotein L gene, or other defective herpes virus vectors (PCT Publication Numbers WO 94/21807 and WO 92/05263); an attenuated adenovirus vector, such as the vector described by Stratford-Perricaudet et al. (J. Clin. Invest., 1992, 90:626-630; see also La Salle et al., Science, 1993, 259:988-990); and a defective adeno-associated virus vector (Samulski et al., J. Virol., 1987, 61:3096-3101; Samulski et al., J. Virol., 1989, 63:3822-3828; Lebkowski et al., Mol. Cell. Biol., 1988, 8:3988-3996).

Various companies produce viral vectors commercially, including, but not limited to, Avigen, Inc. (Alameda, Calif.; AAV vectors), Cell Genesys (Foster City, Calif.; retroviral, adenoviral, AAV vectors, and lentiviral vectors), Clontech (retroviral and baculoviral vectors), Genovo, Inc. (Sharon Hill, Pa.; adenoviral and AAV vectors), Genvec (adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), and Transgene (Strasbourg, France; adenoviral, vaccinia, retroviral, and lentiviral vectors).

Adenoviruses are eukaryotic DNA viruses that can be modified to efficiently deliver a nucleotide of the invention to a variety of cell types. Various serotypes of adenovirus exist. Of these serotypes, preference is given, within the scope of the invention, to using type 2 or type 5 human adenoviruses (Ad 2 or Ad 5) or adenoviruses of animal origin (See, PCT Publication Number WO 94/26914.). Those adenoviruses of animal origin which can be used within the scope of the invention include adenoviruses of canine, bovine, murine (e.g., Mav1, Beard et al., Virology, 1990, 75-81), ovine, porcine, avian, and simian (e.g., SAV) origin. Preferably, the adenovirus of animal origin is a canine adenovirus, more preferably a CAV2 adenovirus (e.g., Manhattan or A26/61 strain, ATCC VR-800, for example). Various replication defective adenovirus and minimum adenovirus vectors have been described (e.g., PCT Publication Numbers WO 94/26914, WO 95/02697, WO 94/28938, WO 94/28152, WO 94/12649, WO 95/02697, WO 96/22378). The replication defective recombinant adenoviruses according to the invention can be prepared by any technique known to the person skilled in the art (e.g., Levrero et al., Gene, 1991, 101:195; European Publication Number EP 185 573; Graham, EMBO J., 1984, 3:2917; Graham et al., J. Gen. Virol., 1977, 36:59). Recombinant adenoviruses are recovered and purified using standard molecular biological techniques, which are well known to one of ordinary skill in the art.

The adeno-associated viruses (AAV) are DNA viruses of relatively small size that can integrate, in a stable and site-specific manner, into the genome of the cells which they infect. They are able to infect a wide spectrum of cells without inducing any effects on cellular growth, morphology, or differentiation, and they do not appear to be involved in human pathologies. The AAV genome has been cloned, sequenced, and characterized. The use of vectors derived from the AAVs for transferring genes in vitro and in vivo has been described (See, PCT Publication Numbers WO 91/18088 and WO 93/09239; U.S. Pat. Nos. 4,797,368 and 5,139,941; European Publication Number EP 488 528). The replication defective recombinant AAVs according to the invention can be prepared by cotransfecting a plasmid containing the nucleic acid sequence of interest flanked by two AAV inverted terminal repeat (ITR) regions, and a plasmid carrying the AAV encapsidation genes (rep and cap genes), into a cell line which is infected with a human helper virus (for example, an adenovirus). The AAV recombinants which are produced are then purified by standard techniques.

In another embodiment, the gene can be introduced in a retroviral vector, e.g., as described in U.S. Pat. No. 5,399,346; Mann et al., Cell, 1983, 33:153; U.S. Pat. Nos. 4,650,764 and 4,980,289; Markowitz et al., J. Virol., 1988, 62:1120; U.S. Pat. No. 5,124,263; European Publication Numbers EP 453 242 and EP178 220; Bernstein et al., Genet. Eng., 1985, 7:235; McCormick, BioTechnology, 1985, 3:689; PCT Publication Number WO 95/07358; and Kuo et al., Blood, 1993, 82:845. The retroviruses are integrating viruses that infect dividing cells. The retrovirus genome includes two LTRs, an encapsidation sequence, and three coding regions (gag, pol and env). In recombinant retroviral vectors, the gag, pol and env genes are generally deleted, in whole or in part, and replaced with a heterologous nucleic acid sequence of interest. These vectors can be constructed from different types of retrovirus, such as, HIV, MoMuLV (“murine Moloney leukaemia virus”), MSV (“murine Moloney sarcoma virus”), HaSV (“Harvey sarcoma virus”), SNV (“spleen necrosis virus”), RSV (“Rous sarcoma virus”), and Friend virus. Suitable packaging cell lines have been described, in particular the cell line PA317 (U.S. Pat. No. 4,861,719), the PsiCRIP cell line (PCT Publication Number WO 90/02806), and the GP+envAm-12 cell line (PCT Publication Number WO 89/07150). In addition, the recombinant retroviral vectors can contain modifications within the LTRs for suppressing transcriptional activity as well as extensive encapsidation sequences which may include a part of the gag gene (Bender et al., J. Virol., 1987, 61:1639). Recombinant retroviral vectors are purified by standard techniques known to those having ordinary skill in the art.

Retroviral vectors can be constructed to function as infectious particles or to undergo a single round of transfection. In the former case, the virus is modified to retain all of its genes except for those responsible for oncogenic transformation properties, and to express the heterologous gene. Non-infectious viral vectors are manipulated to destroy the viral packaging signal, but retain the structural genes required to package the co-introduced virus engineered to contain the heterologous gene and the packaging signals. Thus, the viral particles that are produced are not capable of producing additional virus.

Retrovirus vectors can also be introduced by DNA viruses, which permits one cycle of retroviral replication and amplifies transfection efficiency (See, PCT Publication Numbers WO 95/22617, WO 95/26411, WO 96/39036 and WO 97/19182.).

In another embodiment, lentiviral vectors can be used as agents for the direct delivery and sustained expression of a transgene in several tissue types, including brain, retina, muscle, liver, and blood. The vectors can efficiently transduce dividing and nondividing cells in these tissues, and maintain long-term expression of the gene of interest. For a review, see, Naldini, Curr. Opin. Biotechnol., 1998, 9:457-63; see also, Zufferey et al., J. Virol., 1998, 72:9873-80. Lentiviral packaging cell lines are available and known generally in the art. They facilitate the production of high-titer lentivirus vectors for gene therapy. An example is a tetracycline-inducible VSV-G pseudotyped lentivirus packaging cell line that can generate virus particles at titers greater than 106 IU/ml for at least 3 to 4 days (Kafri et al., J. Virol., 1999, 73: 576-584). The vector produced by the inducible cell line can be concentrated as needed for efficiently transducing non-dividing cells in vitro and in vivo.

In another embodiment, the vector can be introduced in vivo by lipofection, as naked DNA, or with other transfection facilitating agents (peptides, polymers, etc.). Synthetic cationic lipids can be used to prepare liposomes for in vivo transfection of a gene encoding a marker (Felgner et al., Proc. Natl. Acad. Sci. U.S.A., 1987, 84:7413-7417; Feigner and Ringold, Science, 1989, 337:387-388; Mackey et al., Proc. Natl. Acad. Sci. U.S.A., 1988, 85:8027-8031; Ulmer et al., Science, 1993, 259:1745-1748). Useful lipid compounds and compositions for transfer of nucleic acids are described in PCT Patent Publication Numbers WO 95/18863 and WO 96/17823, and in U.S. Pat. No. 5,459,127. Lipids may be chemically coupled to other molecules for the purpose of targeting (see Mackey, et al., supra). Targeted peptides, e.g., hormones or neurotransmitters, and proteins such as antibodies, or non-peptide molecules could be coupled to liposomes chemically.

One can also introduce the vector in vivo as a naked DNA plasmid. Naked DNA vectors for gene therapy can be introduced into the desired host cells by methods known in the art, e.g., electroporation, microinjection, cell fusion, DEAE dextran, calcium phosphate precipitation, use of a gene gun, or use of a DNA vector transporter (e.g., Wu et al., J. Biol. Chem., 1992, 267:963-967; Wu and Wu, J. Biol. Chem., 1988, 263:14621-14624; Canadian Patent Application Number 2,012,311; Williams et al., Proc. Natl. Acad. Sci. USA, 1991, 88:2726-2730). Receptor-mediated DNA delivery approaches can also be used (Curiel et al., Hum. Gene Ther., 1992, 3:147-154; Wu and Wu, J. Biol. Chem., 1987, 262:4429-4432). U.S. Pat. Nos. 5,580,859 and 5,589,466 disclose delivery of exogenous DNA sequences, free of transfection facilitating agents, in a mammal. Recently, a relatively low voltage, high efficiency in vivo DNA transfer technique, termed electrotransfer, has been described (Mir et al., C.P. Acad. Sci., 1988, 321:893; PCT Publication Numbers WO 99/01157; WO 99/01158; WO 99/01175).

Other molecules are also useful for facilitating transfection of a nucleic acid in vivo, such as a cationic oligopeptide (e.g., PCT Patent Publication Number WO 95/21931), peptides derived from DNA binding proteins (e.g., PCT Patent Publication Number WO 96/25508), or a cationic polymer (e.g., PCT Patent Publication Number WO 95/21931), or bupivacaine (U.S. Pat. No. 5,593,972).

The isolated polypeptide of the present invention can be delivered to the mammal using a live vector, in particular using live recombinant bacteria, viruses, or other live agents, containing the genetic material necessary for the expression of the polypeptide or immunogenic fragment as a foreign polypeptide. Particularly, bacteria that colonize the gastrointestinal tract, such as Salmonella, Shigella, Yersinia, Vibrio, Escherichia and BCG have been developed as vaccine vectors, and these and other examples are discussed by Holmgren et al. (1992) and McGhee et al. (1992).

The following might be used as part of a list of RNA vectors, in which one or more of the immunogenic candidate proteins may be inserted.

Classification of Nonsegmented, Negative-Sense, Single Stranded RNA Viruses of the Order Mononegavirales

Family Paramvxoviridae

Subfamily Paramyxovirinae
   Genus Paramyxovirus
      Sendai virus (mouse parainfluenza virus type 1)
      Human parainfluenza virus (PIV) types 1 and 3
      Bovine parainfluenza virus (BPV) type 3
   Genus Rubulavirus
      Simian virus 5 (SV) (Canine parainfluenza virus type 2)
      Mumps virus
      Newcastle disease virus (NDV) (avian Paramyxovirus 1)
      Human parainfluenza virus (PIV-types 2, 4a and 4b)
   Genus Morbillivirus
      Measles virus (MV)
      Dolphin Morbillivirus
      Canine distemper virus (CDV)
      Peste-des-petits-ruminants virus
      Phocine distemper virus
      Rinderpest virus
   Unclassified
      Hendra virus
      Nipah virus
Subfamily Pneumovirinae
   Genus Pneumovirus
      Human respiratory syncytial virus (RSV)
      Bovine respiratory syncytial virus
      Pneumonia virus of mice
   Genus Metapneumovirus
      Human metapneumovirus
      Avian pneumovirus (formerly Turkey rhinotracheitis virus)
         Family Rhabdoviridae
   Genus Lyssavirus
      Rabies virus
   Genus Vesiculovirus
      Vesicular stomatitis virus (VSV)
   Genus Ephemerovirus
      Bovine ephemeral fever virus
         Family Filovirdae
   Genus Filovirus
      Marburg virus

The RNA virus vector is basically an isolated nucleic acid molecule that comprises a sequence which encodes at least one genome or antigenome of a nonsegmented, negative-sense, single stranded RNA virus of the Order Mononegavirales. The isolated nucleic acid molecule may comprise a polynucleotide sequence which encodes a genome, antigenome, or a modified version thereof. In one embodiment, the polynucleotide encodes an operably linked promoter, the desired genome or antigenome, and a transcriptional terminator.

In a preferred embodiment of this invention, the polynucleotide encodes a genome or antigenome that has been modified from a wild-type RNA virus by a nucleotide insertion, rearrangement, deletion, or substitution. The genome or antigenome sequence can be derived from a human or non-human virus. The polynucleotide sequence may also encode a chimeric genome formed from recombinantly joining a genome or antigenome from two or more sources. For example, one or more genes from the A group of RSV are inserted in place of the corresponding genes of the B group of RSV; or one or more genes from bovine PIV (BPIV), PIV-1 or PIV-2 are inserted in the place of the corresponding genes of PIV-3; or RSV may replace genes of PIV and so forth. In additional embodiments, the polynucleotide encodes a genome or anti-genome for an RNA virus of the Order Mononegavirales which is a human, bovine, or murine virus. Since the recombinant viruses formed by the methods of this invention are employed for therapeutic or prophylactic purposes, the polynucleotide may also encode an attenuated or an infectious form of the RNA virus selected. In many embodiments, the polynucleotide encodes an attenuated, infectious form of the RNA virus. In particularly preferred embodiments, the polynucleotide encodes a genome or antigenome of a nonsegmented, negative-sense, single stranded RNA virus of the Order Mononegavirales having at least one attenuating mutation in the 3′ genomic promoter region and having at least one attenuating mutation in the RNA polymerase gene, as described by published International patent application WO 98/13501, which is hereby incorporated by reference.

As vectors, the polynucleotide sequences encoding the modified forms of the desired genome and antigenome as described above also encode one or more genes or nucleotide sequences for the immunogenic proteins of this invention. In addition, one or more heterologous genes may also be included in forming a desired immunogenic composition/vector, as desired. Depending on the application of the desired recombinant virus, the heterologous gene may encode a co-factor, cytokine (such an interleukin), a T-helper epitope, a restriction marker, adjuvant, or a protein of a different microbial pathogen (e.g., virus, bacterium, or fungus), especially proteins capable of eliciting a protective immune response. The heterologous gene may also be used to provide agents which are used for gene therapy. In preferred embodiments, the heterologous genes encode cytokines, such as interleukin-12, which are selected to improve the prophylactic or therapeutic characteristics of the recombinant virus.

Antibodies

The polypeptides of the invention, including the amino acid sequences of even numbered SEQ ID NOS: 2-668, their fragments, and analogs thereof, or cells expressing them, can also be used as immunogens to produce antibodies immunospecific for the polypeptides of the invention. The invention includes antibodies immunospecific for β-hemolytic streptococci and Streptococcus pyogenes polypeptides and the use of such antibodies to detect the presence of, or measure the quantity or concentration of, β-hemolytic streptococci and Streptococcus pyogenes polypeptides in a cell, a cell or tissue extract, or a biological fluid.

The antibodies of the invention include polyclonal antibodies, monoclonal antibodies, chimeric antibodies, and anti-idiotypic antibodies. Polyclonal antibodies are heterogeneous populations of antibody molecules derived from the sera of animals immunized with an antigen. Monoclonal antibodies are a substantially homogeneous population of antibodies to specific antigens. Monoclonal antibodies may be obtained by methods known to those skilled in the art, e.g., Kohler and Milstein, 1975, Nature 256:495-497 and U.S. Pat. No. 4,376,110. Such antibodies may be of any immunoglobulin class including IgG, IgM, IgE, IgA, GILD and any subclass thereof.

Chimeric antibodies are molecules, different portions of which are derived from different animal species, such as those having variable region derived from a murine monoclonal antibody and a human immunoglobulin constant region. Chimeric antibodies and methods for their production are known in the art (Cabilly et al., 1984, Proc. Natl. Acad. Sci. USA 81:3273-3277; Morrison et al., 1984, Proc. Natl. Acad. Sci. USA 81:6851-6855; Boulianne et al., 1984, Nature 312:643-646; Cabilly et al., European Patent Application 125023 (published Nov. 14, 1984); Taniguchi et al., European Patent Application 171496 (published Feb. 19, 1985); Morrison et al., European Patent Application 173494 (published Mar. 5, 1986); Neuberger et al., PCT Application WO 86/01533 (published Mar. 13, 1986); Kudo et al., European Patent Application 184187 (published Jun. 11, 1986); Morrison et al., European Patent Application 173494 (published Mar. 5, 1986); Sahagan et al., 1986, J. Immunol. 137:1066-1074; Robinson et al., PCT/US86/02269 (published May 7, 1987); Liu et al., 1987, Proc. Natl. Acad. Sci. USA 84:3439-3443; Sun et al., 1987, Proc. Natl. Acad. Sci. USA 84:214-218; Better et al., 1988, Science 240:1041-1043). These references are hereby incorporated by reference.

An anti-idiotypic (anti-Id) antibody is an antibody which recognizes unique determinants generally associated with the antigen-binding site of an antibody. An anti-Id antibody is prepared by immunizing an animal of the same species and genetic type (e.g., mouse strain) as the source of the monoclonal antibody with the monoclonal antibody to which an anti-Id is being prepared. The immunized animal will recognize and respond to the idiotypic determinants of the immunizing antibody by producing an antibody to these isotypic determinants (the anti-Id antibody).

Accordingly, monoclonal antibodies generated against the polypeptides of the present invention may be used to induce anti-Id antibodies in suitable animals. Spleen cells from such immunized mice can be used to produce anti-Id hybridomas secreting anti-Id monoclonal antibodies. Further, the anti-Id antibodies can be coupled to a carrier such as keyhole limpet hemocyanin (KLH) and used to immunize additional BALB/c mice. Sera from these mice will contain anti-anti-Id antibodies that have the binding properties of the final mAb specific for a R-PTPase epitope. The anti-Id antibodies thus have their idiotypic epitopes, or “idiotopes” structurally similar to the epitope being evaluated, such as Streptococcus pyogenes polypeptides.

The term “antibody” is also meant to include both intact molecules as well as fragments such as Fab which are capable of binding antigen. Fab fragments lack the Fc fragment of intact antibody, clear more rapidly from the circulation, and may have less non-specific tissue binding than an intact antibody (Wahl et al., 1983, J. Nucl. Med. 24:316-325). It will be appreciated that Fab and other fragments of the antibodies useful in the present invention may be used for the detection and quantitation of Streptococcus pyogenes polypeptides according to the methods for intact antibody molecules.

The anti-Id antibody may also be used as an “immunogen” to induce an immune response in yet another animal, producing a so-called anti-anti-Id antibody. The anti-anti-Id may be epitopically identical to the original mAb which induced the anti-Id. Thus, by using antibodies to the idiotypic determinants of a mAb, it is possible to identify other clones expressing antibodies of identical specificity.

The antibodies are used in a variety of ways, e.g., for confirmation that a protein is expressed, or to confirm where a protein is expressed. Labeled antibody (e.g., fluorescent labeling for FACS) can be incubated with intact bacteria and the presence of the label on the bacterial surface confirms the location of the protein, for instance.

Antibodies generated against the polypeptides of the invention can be obtained by administering the polypeptides or epitope-bearing fragments, analogs, or cells to an animal using routine protocols. For preparing monoclonal antibodies, any technique which provides antibodies produced by continuous cell line cultures are used.

Immunogenic Compositions

Also provided are immunogenic compositions. The immunogenic compositions of the present invention can be used for the treatment of streptococcal infections in mammals, such as humans (preferably) and non-human animals. For example, the animals may be bovine, canine, equine, feline, and porcine. It is noted that SEQ ID NO: 415 (ORF 1021) corresponds to a protein which also appears in S. equi. Accordingly, this sequence can be used in immunogenic compositions for treating equine infections, as well as in other animals or humans. Particular applications include, but are not limited to, the treatment of strangles, a highly contagious disease of the nasopharynx and draining lymph nodes of Equidae, and the treatment of respiratory infections and mastitis in bovines, equines, and swine.

The immunogenic compositions of the invention may either be prophylactic (i.e., to prevent infection or reduce the onset of infection) or therapeutic (i.e., to treat a disease or side effects caused by an infection after the infection).

The immunogenic compositions may comprise a polypeptide of the invention. To do so, one or more polypeptides are adjusted to an appropriate concentration and can be formulated with any suitable adjuvant, diluent, carrier, or any combination thereof. Physiologically acceptable media may be used as carriers and/or diluents. These include, but are not limited to, water, an appropriate isotonic medium, glycerol, ethanol and other conventional solvents, phosphate buffered saline, and the like.

As used herein, an “adjuvant” is a substance that serves to enhance the immunogenicity of an antigen, whether it is a polypeptide or a polynucleotide. Thus, adjuvants are often given to boost the immune response and are well known to the skilled artisan. Suitable adjuvants include, but are not limited to, aluminum salts (alum), such as aluminum phosphate and aluminum hydroxide, Mycobacterium tuberculosis, Bordetella pertussis, bacterial lipopolysaccharides, aminoalkyl glucosamine phosphate compounds (AGP), or derivatives or analogs thereof, which are available from Corixa (Hamilton, Mont.), and which are described in U.S. Pat. No. 6,113,918, which is hereby incorporated by reference. One such AGP is 2-ethyl 2-Deoxy-4-O-phosphono-3-O-2-b-D-glucopyranoside, which is also known as 529 (formerly known as RC529). This 529 adjuvant is formulated as an aqueous form or as a stable emulsion. Other adjuvants are MPL® (3-O-deacylated monophosphoryl lipid A) (Corixa) described in U.S. Pat. No. 4,912,094, synthetic polynucleotides such as oligonucleotides containing a CpG motif (U.S. Pat. No. 6,207,646, saponins such as Quil A or STIMULON® QS-21 (Antigenics, Framingham, Mass.), described in U.S. Pat. No. 5,057,540, a pertussis toxin (PT), or an E. coli heat-labile toxin (LT), particularly LT-K63, LT-R72, CT-S 109, PT-K9/G129; see, e.g., International Patent Publication Nos. WO 93/13302 and WO 92/19265, cholera toxin (either in a wild-type or mutant form, for example, wherein the glutamic acid at amino acid position 29 is replaced by another amino acid, preferably a histidine, in accordance with published International Patent Application number WO 00/18434).

Various cytokines and lymphokines are suitable for use as adjuvants. One such adjuvant is granulocyte-macrophage colony stimulating factor (GM-CSF), which has a nucleotide sequence as described in U.S. Pat. No. 5,078,996, which is hereby incorporated by reference. A plasmid containing GM-CSF cDNA has been transformed into E. coli and has been deposited with the American Type Culture Collection (ATCC), 10801 University Boulevard, Manassas, Va. 20110-2209, under Accession Number 39900. The cytokine Interleukin-12 (IL-12) is another adjuvant which is described in U.S. Pat. No. 5,723,127, which is hereby incorporated by reference. Other cytokines or lymphokines have been shown to have immune modulating activity, including, but not limited to, the interleukins 1-alpha, 1-beta, 2, 4, 5, 6, 7, 8, 10, 13, 14, 15, 16, 17 and 18, the interferons-alpha, beta and gamma, granulocyte colony stimulating factor, and the tumor necrosis factors alpha and beta, and are suitable for use as adjuvants.

The polypeptide can also include at least a portion of the polypeptide, optionally conjugated or linked to a peptide, polypeptide, or protein, or to a polysaccharide.

The immunogenic compositions of the invention can further include immunogenic conjugates as disclosed in U.S. Pat. Nos. 4,673,574, 4,902,506, 5,097,020, and 5,360,897 (assigned to The University of Rochester), hereby incorporated by reference. These patents teach immunogenic conjugates which are the reductive amination product of an immunogenic capsular polymer fragment having a reducing end and derived from a bacterial capsular polymer of a bacterial pathogen, and a bacterial toxin or toxoid. The present invention also includes immunogenic compositions containing these conjugates which elicit effective levels of anti-capsular polymer antibodies in humans.

Combination immunogenic compositions are provided by including two or more of the polypeptides of the invention, as well as by combining one or more of the polypeptides of the invention with one or more known Streptococcus pyogenes polypeptides, including, but not limited to, the C5a peptidase, the M proteins, adhesins, and the like.

The immunogenic compositions of the invention also comprise a polynucleotide sequence of the invention operatively associated with a regulatory sequence that controls gene expression. The polynucleotide sequence of interest is engineered into an expression vector, such as a plasmid, under the control of regulatory elements which will promote expression of the DNA, that is, promoter and/or enhancer elements. In a preferred embodiment, the human cytomegalovirus immediate-early promoter/enhancer is used (U.S. Pat. No. 5,168,062). The promoter may be cell-specific and permit substantial transcription of the polynucleotide only in predetermined cells.

The polynucleotide is introduced directly into the host either as “naked” DNA (U.S. Pat. No. 5,580,859) or formulated in compositions with agents which facilitate immunization, such as bupivacaine and other local anesthetics (U.S. Pat. No. 5,593,972) and cationic polyamines (U.S. Pat. No. 6,127,170).

In this polynucleotide immunization procedure, the polypeptides of the invention are expressed on a transient basis in vivo; no genetic material is inserted or integrated into the chromosomes of the host. This procedure is to be distinguished from gene therapy, where the goal is to insert or integrate the genetic material of interest into the chromosome. An assay is used to confirm that the polynucleotides administered by immunization do not give rise to a transformed phenotype in the host (U.S. Pat. No. 6,168,918).

Once formulated, the immunogenic compositions of the invention can be administered directly to the subject, delivered ex vivo to cells derived from the subject, or in vitro for expression of recombinant proteins. For delivery directly to the subject, administration may be by any conventional form, such as intranasally, parenterally, orally, intraperitoneally, intravenously, subcutaneously, or topically applied to any mucosal surface such as intranasal, oral, eye, lung, vaginal, or rectal surface, such as by an aerosol spray.

The subjects can be mammals or birds. The subject can also be a human. An immunologically effective amount of the immunogenic composition in an appropriate number of doses is administered to the subject to elicit an immune response. Immunologically effective amount, as used herein, means the administration of that amount to a mammalian host (preferably human), either in a single dose or as part of a series of doses, sufficient to at least cause the immune system of the individual treated to generate a response that reduces the clinical impact of the bacterial infection. Protection may be conferred by a single dose of the immunogenic composition, or may require the administration of several doses, in addition to booster doses at later times to maintain protection. This may range from a minimal decrease in bacterial burden to prevention of the infection. Ideally, the treated individual will not exhibit the more serious clinical manifestations of the β-hemolytic streptococcal infection. The dosage amount can vary depending upon specific conditions of the individual, such as age and weight. This amount can be determined in routine trials by means known to those skilled in the art.

Various tests are used to assess the in vitro immunogenicity of the polypeptides of the invention. For example, the polypeptides can be expressed recombinantly or chemically synthesized and used to screen subject sera by immunoblot. A positive reaction between the subject and subject serum indicates that the subject has previously mounted an immune response to the polypeptide in question, i.e., the polypeptide is an immunogen. This method can also be used to identify immunodominant polypeptides.

An ELISA assay is also used to assess in vitro immunogenicity, wherein the polypeptide antigen of interest is coated onto a plate, such as a 96 well plate, and test sera from either a vaccinated or naturally exposed animal (e.g., human) is reacted with the coating antigen. If any antibody, specific for the test polypeptide antigen, is present, it can be detected by standard methods known to one skilled in the art.

Alternatively, the same sera can be reacted with whole Streptococcus pyogenes cells. Reactive antibody present in the sera can then be detected using a colloidal gold conjugated antibody and visualized by LV-SEM.

Efficacy of vaccine antigens can be tested using two animal challenge assay models. The first addresses mucosal immunity. Mice are actively immunized, parenterally or mucosally, with the vaccine candidates following established procedures. The mice are then challenged with wild-type Streptococcus pyogenes by intranasal administration. Streptococcus pyogenes persistence in the nasal/pharyngeal cavity of the mice can then be measured by standard techniques. Efficacy is reflected by an enhanced clearance of the bacteria from the throats of the animals.

Alternatively, subsequent to active parenteral immunization, protection against systemic infection can be evaluated by subcutaneous injection of Streptococcus pyogenes cells. Efficacy is measured by reduction in death and/or reduced histopathology at the site of injection.

Detection in a Sample

Also provided are methods for detecting and identifying β-hemolytic Streptococcus and Streptococci pyogenes in a biological sample. In one embodiment, the method comprises the steps of (a) contacting the biological sample with a polynucleotide of the invention under conditions that permit hybridization of complementary base pairs and (b) detecting the presence of hybridization complexes in the sample. In another embodiment, the method comprises the steps of (a) contacting the biological sample with an antibody of the invention under conditions suitable for the formation of immune complexes and (b) detecting the presence of immune complexes in the sample. In yet another embodiment, the method comprises the steps of (a) contacting the biological sample with a polypeptide of the invention under conditions suitable for the formation of immune complexes and (b) detecting the presence of immune complexes in the sample.

Antigens, or antigenic fragments thereof, of the invention are used in immunoassays to detect antibody levels or, conversely, anti-Streptococcus pyogenes antibodies are used to detect antigen levels. Immunoassays based on well defined, recombinant antigens can be developed to replace invasive diagnostic methods. Antibodies to the polypeptides of the invention within biological samples, including, for example, blood or serum samples, can be detected. Protocols for the immunoassay may be based, for example, upon competition, or direct reaction, or sandwich type assays. Protocols may also, for example, use solid supports, or may be by immunoprecipitation. The polypeptides of the invention can also be a useful in receptor-ligand studies.

The following examples are illustrative and the present invention is not intended to be limited thereto.

### EXAMPLE 1

Bacteria, Media, and Reagents

E. coli was cultured and maintained in SOB (0.5% Yeast Extract, 2.0% Tryp, 10 mM Sodium Chloride, 2.5 mM Potassium Chloride, 10 mM Magnesium Chloride, 10 mM Magnesium Sulfate)containing the appropriate antibiotic. Ampicillin was used at a concentration of 100 μg/mL, chloramphenicol at 30 μg/mL, and kanamycin at 50 μg/mL. The Streptococcus pyogenes strain SF370 (ATCC accession number 700294) was cultured in 30 g/L Todd Hewitt, 5 g/L yeast extract (THY) broth.

Bioinformatics/Gene Mining

The genomic, unannotated sequence of Streptococcus pyogenes M1 strain was downloaded from the website of the University of Oklahoma and was analyzed to identify open reading frames (ORFs). This genomic sequence was reported as being submitted to GenBank and assigned accession number AE004092, and strain M1 GAS was reported as being submitted to the ATCC and given accession number ATCC 700294.

An ORF was defined as having either one of three potential start site codons, ATG, GTG, or TTG and either one of three potential stop codons, TAA, TAG, TGA. A unique set of three ORF finder algorithms was used to enhance the efficiency for determining all ORFs: GLIMMER (59); GeneMark (34); and a third algorithm developed by inventor's assignee.

In order to evaluate the accuracy of the ORFs determined, a discrete mathematical cosine function, known in the art has a discrete cosine transformation (DiCTion), was employed to assign a score for each ORF. An ORF with a DiCTion score >1.5 is considered to have a high probability of encoding a protein product. The minimum length of an ORF predicted by the three ORF finding algorithms was set to 225 nucleotides (including stop codon) which would encode a protein of 74 amino acids.

As a final search for remnants of ORFs, all noncoding regions >75 nucleotides were searched against the public protein databases (described below) using tBLASTn. This helped to identify regions of genes that contained frameshifts (42) or fragments of genes that might have a role in causing antigenic variation (21). Any remnant ORFs found here were added to the ORF database of Streptococcus pyogenes. An in-house graphical analysis program was used to show all six reading frames and the location of the predicted ORFs relative to the genomic sequence. This helped to eliminate those ORFs that had large overlaps with other ORFs, although there are known cases of ORFs being totally embedded within other ORFs (25, 33).

The initial annotation of the Streptococcus pyogenes ORFs was performed using the BLAST v. 2.0 Gapped search algorithm, BLASTp, to identify homologous sequences. A cutoff “e” value of anything <e⁻¹⁰ was considered significant. Other search algorithms, including FASTA and PSI-BLAST, were also used. The non-redundant protein sequence databases used for the homology searches consisted of GenBank, SWISS-PROT, PIR, and TREMBL database sequences updated daily. ORFs with a BLASTp result of >e⁻¹⁰ were considered to be unique to Streptococcus pyogenes.

A keyword search of the entire Blast results was carried out using known or suspected vaccine target genes as well as words that identified the location of a protein or function. Additionally, a keyword search was performed of all MEDLINE references associated with the initial Blast results to look for additional information regarding the ORFs.

For DNA analysis, the % G+C content within each gene was identified. The % G+C content of an ORF was calculated as the (G+C) content of the third nucleotide position of all the codons within an ORF. The value reported was the difference of this value from the arithmetic mean of such values obtained for all ORFs found in the organism. Any absolute value ≧8 was considered important for further analysis, as these ORFs may have arisen from horizontal transfer as has been shown in the case of cag pathogenicity island from H. pylori (2), a pattern in keeping with many other pathogenicity islands (22).

Several parameters were used to determine partitioning of the predicted proteins. Proteins destined for translocation across the cytoplasmic membrane encode a leader signal (also called signal sequence) composed of a central hydrophobic region flanked at the N-terminus by positively charged residues (56). The program SignalP was used to identify signal peptides and their cleavage sites (46). To predict protein localization in bacteria, the software PSORT was used (44). This program uses a neural net algorithm to predict localization of proteins to the cytoplasm, periplasm, and cytoplasmic membrane for Gram-positive bacteria as well as outer membrane for Gram-negative bacteria. Transmembrane (TM) domains of proteins were analyzed using the software program TopPred2 (10). This program predicts regions of a protein that are hydrophobic that may potentially span the lipid bilayer of the membrane. Outer membrane proteins typically do not have an α-helical TM domain.

The Hidden Markov Model (HMM) Pfam database of multiple alignments of protein domains or conserved protein regions (61) was used to identify Streptococcus pyogenes proteins that may belong to an existing protein family. Keyword searching of this output was used to help identify surface localized Streptococcus pyogenes proteins that might have been missed by the Blast search criteria. HMM models were also developed by inventor's assignee. A computer algorithm, HMM Lipo, was developed to predict lipoproteins using 132 biologically characterized non-Streptococcus pyogenes bacterial lipoproteins from over 30 organisms. This training set was generated from experimentally proven prokaryotic lipoproteins. The protein sequence from the start of the protein to the cysteine amino acid plus the next two additional amino acids were used to generate the HMM. Using about 70 known prokaryotic proteins containing the LPXTG cell wall sorting signal, a HMM (15) was developed to predict cell wall proteins that are anchored to the peptidoglycan layer (38, 45). The model used not only the LPXTG sequence, but also included two features of the downstream sequence, the hydrophobic transmembrane domain and the positively charged carboxy terminus. There are also a number of proteins that interact, non-covalently, with the peptidoglycan layer and are distinct from the LPXTG protein class described above. These proteins seem to have a consensus sequence at their carboxy terminus (32). A HMM of this region was developed and used to identify Streptococcus pyogenes proteins falling into this class.

The proteins encoded by Streptococcus pyogenes identified ORFs were also evaluated for other characteristics. A tandem repeat finder (5) identified ORFs containing repeated DNA sequences such as those found in MSCRAMMs (20) and phase variable surface proteins of Neisseria meningitidis (51). Proteins that contain the Arg-Gly-Asp (RGD) attachment motif, together with integrins that serve as their receptor, constitute a major recognition system for cell adhesion. RGD recognition is one mechanism used by microbes to gain entry into eukaryotic tissues (29, 63). However, not all RGD-containing proteins mediate cell attachment. It has been shown that RGD-containing peptides with a proline at the carboxy end (RGDP) are inactive in cell attachment assays (52) and, hence, were excluded. Geanfammer software was used to cluster proteins into homologous families (50). Preliminary analysis of the family classes provided novel ORFs within a vaccine candidate cluster as well as defining potential protein function.

Tryptic Digestion of Streptococcus pyogenes

A starter culture of Streptococcus pyogenes was grown overnight in THY at 37° C., in 5% CO₂, or in atmospheric O₂. Each starter culture was then diluted 1:25 in 200 mL fresh THY, and grown to an OD₄₉₀ of 1-1.3, in either CO₂ or atmospheric O₂, respectively. The cells were then harvested by centrifugation at 4,000× g, for 15 min., and washed three times in 10 mL 20 mM Tris, pH 8.0, 150 mM NaCl buffer. Following the last wash, each pellet was resuspended in 2 mL same buffer containing 0.8 M sucrose and distributed equally between two tubes. To one tube of each growth condition, 40 μg trypsin was added; the other tube was used as a negative digestion control. The cell suspensions were rocked at 37° C. for 4 hours. A sample of each suspension was taken for viable cell counts and visualization by low-voltage scanning electron microscopy (LV-SEM). The suspensions were then centrifuged and the supernatants were collected and filtered through a low protein binding, 2 μM filter.

Micro-Capillary HPLC Interface

Peptide extracts were analyzed on an automated microelectrospray reversed phase HPLC. The microelectrospray interface consisted of a Picofrit fused silica spray needle, 50 cm length by 75 um ID, 8 μm orifice diameter (New Objective, Cambridge, Mass.) packed with 10 μm C18 reversed-phase beads (YMC, Wilmington, N.C.) to a length of 10 cm. The Picofrit needle was mounted in a fiber optic holder (Melles Griot, Irvine, Calif.) held on a base positioned at the front of the mass spectrometer detector. The rear of the column was plumbed through a titanium union to supply an electrical connection for the electrospray interface. The union was connected with a length of fused silica capillary (FSC) tubing to a FAMOS autosampler (LC-Packings, San Francisco, Calif.) that was connected to an HPLC solvent pump (ABI 140C, Perkin-Elmer, Norwalk, Conn.). The HPLC solvent pump delivered a flow of 50 μL/min. which was reduced to 250 nL/min. using a PEEK microtight splitting tee (Upchurch Scientific, Oak Harbor, Wash.), and then delivered to the autosampler using an FSC transfer line. The LC pump and autosampler were each controlled using their internal user programs. Samples were inserted into plastic autosampler vials, sealed, and injected using a 5 μl sample loop.

Microcapillary HPLC-Mass Spectrometry

Extracted peptides from the surface digests were concentrated 10-fold using a Savant Speed Vac Concentrator (ThermoQuest, Holdbrook, N.Y.), and then were separated by the microelectrospray HPLC system using a 50 min. gradient of 0-50% solvent B (A: 0.1M HoAc, B: 90% MeCN/0.1M HoAc). Peptide analyses were conducted on a Finnigan LCQ-DECA ion trap mass spectrometer (ThermoQuest, San Jose, Calif.) operating at a spray voltage of 1.5 kV, and using a heated capillary temperature of 125° C. Data were acquired in automated MS/MS mode using the data acquisition software provided with the instrument. The acquisition method included 1 MS scan (375-600 m/z) followed by MS/MS scans of the top 2 most abundant ions in the MS scan. The instrument then conducted a second MS scan (600-1000 m/z) followed by MS/MS scans of the top 2 most abundant ions in that scan. The dynamic exclusion and isotope exclusion functions were employed to increase the number of peptide ions that were analyzed (settings: 3 amu=exclusion width, 3 min.=exclusion duration, 30 sec=pre-exclusion duration, 3 amu=isotope exclusion width).

Data Analysis

Automated analysis of MS/MS data was performed using the SEQUEST computer algorithm incorporated (17) into the Finnigan Bioworks data analysis package (ThermoQuest, San Jose, Calif.) using the database of proteins derived from the complete genome of Streptococcus pyogenes.

Cloning and Protein Expression

Primer sets were designed for PCR amplification of desired ORFs such that the forward 5′ primer would anneal at the start of the predicted mature protein. For lipoproteins, the 5′ forward primer was designed to anneal just after the codon encoding a cysteine residue of the mature protein to minimize disulfide bridging. Design of the opposing reverse 3′ primers was dependent upon the type of predicted protein. For those proteins that contained an LPXTG, the primer was designed such that it would anneal at the beginning (5′ end) of the cell wall anchor region. For all other predicted proteins, they were designed such that they would anneal at the 3′ end of the ORF. Additionally, the 5′-forward primer was initially designed to allow an in-frame fusion to thioredoxin with the opposing 3′-reverse primer allowing read-through to include a downstream his-patch and V5 epitope (pBAD/thio-TOPO®, Invitrogen, Carlsbad, Calif.). The pBAD vector uses an arabinose inducible promoter. In parallel, these same PCR products were also cloned into pCRT7 TOPO® (Invitrogen, Carlsbad, Calif.). This allowed for an N-terminal fusion to an Xpress epitope and a his-tag for purification.

All PCR reactions used the Streptococcus pyogenes M1 strain, SF370 (ATCC accession number 700294), as the template. PCR products were transformed into the E. coli host, TOP10, and plated on SOB containing 100 μg/mL ampicillin. Colonies were screened by PCR amplification using a vector specific 5′ primer and the specific 3′ reverse primer annealing to the gene insert. Colonies were seeded into wells of a 96 well microtiter plates containing 50 μL 50% glycerol. 10-12 colonies per gene were seeded in one row of the plate. In a second 96 well PCR plate, 50 μL reactions were set up specific to the gene of interest. One μL of the cells suspended in glycerol was used as template in the PCR reaction. Reactions that produced bands of the expected size were analyzed further. The cells that were seeded in 50% glycerol had SOB media added to them and were incubated at 37° C. for 5-8 hours and frozen at −70° C.

PCR positive colonies were inoculated into 2 mL cultures for overnight growth. Part of the culture was used to prepare plasmid DNA that was analyzed by restriction digest to confirm the inserts while another part was used to seed 10 mL expression cultures (for pBAD plasmids) for expression. Mid-log phase cultures were induced with 0.5% L-arabinose for 2 hours. T7/NT plasmids were transformed into the expression strain BLR(DE3) pLysS before screening. T7/NT cultures were induced by the addition of 1 mM IPTG and incubated for 2 hours. Whole cell lysates of induced cultures were run on SDS-PAGE in duplicate. One gel was stained with coomassie and the other was transferred to nitrocellulose and probed with antibody to the relevant epitope tag.

Positive clones were grown in 1-2 L volumes and induced for large-scale purification. Solubility and expression level of the recombinant proteins were assessed by freeze-thaw lysis of the cells followed by DNase/RNase digestion and centrifugation at 9,000× g for 15 min. in a RC5B refrigerated centrifuge (sorbol®, Dupont, Wilmington, Del.). The soluble fraction was removed from the insoluble material and both were separated and evaluated for protein localization and expression by SDS-PAGE. Soluble fusion proteins were purified by passing the soluble fraction of lysed cells over Ni-NTA (Qiagen Inc., Valencia, Calif.) resin and eluting the bound proteins with imidazole. Eluted proteins were buffer exchanged on PD-10 columns (Amersham Pharmacia Biotech, Piscataway, N.J.).

Insoluble recombinant proteins were washed and centrifuged 3 times in PBS, 0.1% TRITON-X100. The inclusion bodies were then solubilized in PBS 4 M urea and buffer exchanged through a PD-10 column (Amersham Pharmacia, Piscataway, N.J.) into PBS, 0.01% TRITON-X100, 0.5 M NaCl. Protein was quantitated by the Lowry assay and checked for purity and concentration by SDS-PAGE.

Generation of Polyclonal Antisera

Swiss Webster mice (5 per group) were immunized at weeks 0, 3, and 5 with 5 μg purified protein prepared above, 100 μg AlPO₄, and 50 μg MPL®, and were then bled at week 8.

Immunogold Labeling of Streptococcus pyogenes and LV-SEM

Bacterial cells were labeled as previously described (49). Briefly, late-log phase bacterial cultures were washed twice, and resuspended to a concentration of 1×10⁸ cells/ml in 10 mM phosphate buffered saline (PBS) (pH 7.4) and placed on poly-L-lysine coated glass coverslips. Excess bacteria were gently washed from the coverslips and unlabeled samples were placed into fixative (2.0% glutaraldehyde, in a 0.1 M sodium cacodylate buffer containing 7.5% sucrose) for 30 min. Bacteria to be labeled with colloidal gold were washed with PBS containing 0.5% bovine serum albumin, and the pre-immune or hyper-immune mouse polyclonal antibody prepared above was applied for 1 hour at room temperature. Bacteria were then gently washed, and a 1:6 dilution of goat anti-mouse conjugated to 18 nm colloidal gold particles (Jackson ImmunoResearch Laboratories, Inc., West Grove, Pa.) was applied for 10 min. at room temperature. Finally, all samples were washed gently with PBS, and placed into the fixative described above. The fixative was washed from samples twice for 10 min. in 0.1 M sodium cacodylate buffer, and postfixed for 30 min. in 0.1 M sodium cacodylate containing 1% osmium tetroxide. The samples were then washed twice with 0.1 M sodium cacodylate, dehydrated with ethanol, critical point dried by the CO₂ method of Anderson using a Samdri-780A (Tousimis, Rockville, Md.), and coated with a 1-2 nm discontinuous layer of platinum. Streptococcus pyogenes cells were viewed with a LEO 1550 field emission scanning electron microscope operated at low accelerating voltages (1-4.5 keV) using a secondary electron detector for conventional topographical imaging and a high-resolution Robinson backscatter detector to enhance the visualization of colloidal gold by atomic number contrast.

### EXAMPLE 2

Immunization and Challenge

Parenteral immunization of mice

Six-week old, female CD1 (Charles River Breeding Laboratories, Inc., Wilmington, Mass.) or Swiss Webster (Taconic Farms Inc., Germantown, N.Y.) mice are immunized at weeks 0, 4, and 6 with 5 μg protein of interest mixed with 50 μg MPL® (Corixa, Hamilton, Mont.) and 100 μg AlPO₄ per dose to a final volume of 200 μL in saline and then injected subcutaneously (s.c.) into mice. Control mice are injected with 5 μg tetanus toxoid mixed with same adjuvants. All mice are bled seven days after the last boosting; sera are then isolated and stored at −20° C.

Mouse Intranasal Challenge Model

Ten days after last immunization, sixteen-hour cultures of challenge Streptococcus pyogenes strains (1×10⁸ to 9×10⁸ colony forming units (CFU)), grown in Todd-Hewitt/Yeast broth containing 20% normal rabbit serum and resuspended in 10 ml of PBS, are administered intranasally to 25 g female CD1 (Charles River Breeding Laboratories, Inc., Wilmington, Mass.) or Swiss Webster (Taconic Farms Inc., Germantown, N.Y.) mice. Viable counts are determined by plating dilutions of cultures on blood agar plates.

Each mouse is anesthetized with 1.2 mg of ketamine HCl (Fort Dodge Animal Health, Ft. Dodge, Iowa) by i.p. injection. The bacterial suspension is inoculated to the nostril of anesthetized mice (10 μL per mouse). Sixteen hours after challenge, mice are sacrificed, the noses are removed and homogenized in 3-ml sterile saline with a tissue homogenizer (Ultra-Turax T25, Janke & Kunkel Ika-Labortechnik, Staufen, Germany). The homogenate is 10-fold serially diluted in saline and plated onto blood agar plates containing 200 mg of streptomycin per ml. After overnight incubation at 37° C., β-hemolytic colonies on plates are counted. All challenge strains are marked by streptomycin resistance to distinguish them from β-hemolytic bacteria that may persist in the normal flora.

Subcutaneous Mouse Challenge Model

Five-week-old (20- to 30-g) outbred, immunocompetent, hairless male mice (strain Crl:SKH1-hrBR) (Charles River, Wilmington, Mass.) are used for subcutaneous injection. Tissue samples are collected following humane euthanasia.

Streptococcus pyogenes cells, grown as described in Example 1, are harvested and washed once with sterile ice-cold, pyrogen-free phosphate-buffered saline (PBS). The optical density at 600 nm (OD₆₀₀) is adjusted to give the required inoculum. Streptococcus pyogenes (1×10⁸ CFU) contained in 0.1 ml are injected subcutaneously in the right flank of each animal with a tuberculin syringe. Control mice are treated with the same volume of PBS. The number of CFU inoculated per mouse is verified for each experiment by colony counts on tryptose agar plates containing 5% sheep blood (Becton Dickinson, Cockeysville, Md.). The mice are observed for 21 days after challenge. Blood is collected from each dead animal by cardiac puncture and cultured on blood agar plates.

Tissue Collection and Histology

Prior to inoculation, the animals are assigned to groups with a random number generator, and blood samples are drawn to establish baseline hematologic data. Blood and tissue samples are collected at 24, 48, and 72 h after inoculation. The methods used for blood and tissue collection are identical for all time points.

Blood samples are obtained from the retro-orbital sinus of the animals, and complete blood count analysis is performed with a Technicon H*1 (Tarrytown, N.Y.) hematology analyzer with species-specific software. Skin samples are collected by wide marginal excision around the abscess or the injection site. These samples always include tissue from the injection site and contiguous grossly normal tissue for comparison. Care is taken to preserve the anatomic orientation of the samples. Tissue samples are also obtained from the heart, liver, spleen, and lung.

All tissues are fixed in 10% neutral buffered formalin supplemented with zinc chloride (Antech, Ltd., Battle Creek, Mich.). Whole lungs are first infused with formalin and then, along with the other organs, fixed by submersion. The samples are placed in formalin for 18 to 24 h and then transferred to 70% ethyl alcohol prior to processing. Standard histologic methods of dehydration in ascending grades of ethyl alcohol, clearing in xylene, and paraffin infiltration are employed. The paraffin blocks are processed with a rotary microtome to obtain 4-μm sections. The histologic sections are stained with hematoxylin and eosin and mounted. Selected tissues are sectioned and stained with a tissue Gram stain.

Mouse Measurements

Mice are weighed immediately before GAS inoculation. The animal weight and abscess sizes are measured 12 h after inoculation and daily thereafter for the first week. Animals are then observed at weekly intervals for a total of 21 days. The dimensions of the abscesses are measured with a caliper; length (L) and width (W) values were used to calculate abscess volume [V=4/3π(L/2)²×(W/2)] and area [A=π(L/2)×(W/2)], employing equations for a spherical ellipsoid.

### EXAMPLE 3

Seventy-seven ORFs were initially selected for characterization by “wet chemistry”. Aspects of these studies included: 1) the ability of specific mouse polyclonal sera generated against each purified protein to react to the surface of the bacterium as measured by whole-cell ELISA, 2) the ability of these same sera to react to the bacterial cell surface during log phase or stationary phase growth as determined by LV-SEM, 3) the genetic conservation of the genes across strains (M serotypes) of S. pyogenes as well as other species of streptococci that include the groups C and G, 4) phenotypic expression of specific proteins by these strains as determined by dot blot, 5) expression of the genes of interest at the transcriptional level by quantitative PCR (qPCR), and 6) the ability of human antibody to these proteins to be opsonic in an in vitro opsonophagocytic assay.

Seventy-four of the ORFs have been cloned and expressed in E. coli, and 62 of the expressed proteins have been purified. These purified proteins were injected into mice for the generation of the specific antibody for which the analysis by whole-cell ELISA and LV-SEM has been completed. Additionally, 24 ORFs have been evaluated for genetic conservation across S. pyogenes strains and streptococcal species; a few have been evaluated for expression at the transcriptional level by qPCR in vitro and in vivo. Lastly, human antibody specific for S. pyogenes proteins has been purified and evaluated in opsonophagocytic assays.

Whole-Cell Enzyme-Linked Immunosorbent Assay (ELISA)

S. pyogenes strain SF-370 was used to inoculate Todd-Hewitt broth containing 0.5% yeast extract (THY), and was cultured overnight at 37° C. Cells were harvested by centrifugation and washed two times with phosphate buffered saline (PBS). The bacteria were resuspended in PBS to an OD₆₀₀ of 0.2 with PBS and each well of a 96 well polystyrene microtiter plate was coated with 100 μl of the bacterial suspension. The plates were then air-dried at room temperature, sealed with a mylar plate sealer and stored at 4° C. inverted for up to three months. In preparation for the assay, the plates were washed three times with Tris Buffered Saline (TBS)/0.1% Brij-35, 100 μl/well of ORF-specific antisera was added to each well, and incubated at 37° C. for two hours. The plates were then washed three times with TBS/0.1% Brij-35, 100 μl/well of the secondary antibody conjugate was added to each well, and incubated for one hour at room temperature. Finally, after three washes with PBS, 100 μl/well of the substrate was added to each well and allowed to develop for 60 minutes at room temperature. The reaction was then stopped by adding 50 μl/well of 3N NaOH. Absorbance values (OD₄₀₅) were determined using an ELISA plate reader.

Polymerase Chain Reaction (PCR) Analysis of Genetic Conservation

The bacterial strains tested included ten from S. pyogenes, SF370 (M1), 90-226 (M1), 80-003 (M1), CS210 (M2), CS194 (M4), 83-112 (M5), CS204 (OF+, M11, T11), CS24 (M12), 95-0061 (M28), CS101 (M49), and a fourth M1 serotype SpeB+, two S. zooepidemicus strains, CS258 and GB21, and three group G streptococcal strains, CS241, CS140, and CS242. Five ml overnight cultures were grown in THY. Two and one/half ml of each culture were centrifuged and resuspended in 480 μl of 50 mM EDTA, 120 μl of 10 mg/ml lysozyme and 2 μl of 2500 unit/ml mutanolysin. Samples were incubated at 37° C. for one hour. Promega's Wizard Genomic DNA Purification Kit was followed for the remainder of the genomic purifications. Primer sets for the full-length genes and secondly, primers designed for qPCR (see below) were used in the assay. PCR cycling conditions are as follows: 94° C. hold for one minute, 16 cycles of 94° C. for 15 seconds and 58° C. for 10 min, 12 cycles, each increasing 15 seconds from the previous, of 94° C. for 15 seconds and 58° C. for 10 min, a ten minute hold at 72° C., and finally a 4° C. hold. PCR products were verified by mobility in agarose gels. Any amplification containing an intense band of the appropriate size was considered to be a positive result.

Quantitative PCR (qPCR)

RNA was isolated from bacterial cultures described above or from infected homogenized mouse tissue. Samples were suspended in 2 ml RNAlater (Ambion, Austin, Tex., USA) and quick-frozen using dry-ice/ethanol and stored at −70° C. until use. Samples were thawed to room temperature and then frozen again using the above method, for a total of three freeze-thaw cycles. Samples were either treated with 100 μl 10 mg/ml lysozyme and 10 μl 2500 unit/ml mutanolysin, and incubated at 37° C. for one hour, or samples were mixed with an equal volume of 0.1 mm glass beads and placed into the bead beater for one minute at 4800 rpm to lyse the cells. Supernatant was recovered from the beads and an additional 400 μl RNAlater was added to the beads and mixed as above. Supernatants recovered from beads or digested solution were mixed with an equal volume of RNAqueous Lysis/Binding Solution (Ambion) and vortexed vigorously. Samples were spun at top speed in a microcentrifuge for two minutes to pellet any remaining tissue. The supernatants were mixed with an equal volume of 64% ethanol and passed through a filter cartridge, 700 μl at a time. Filter cartridges were washed as described in the RNAqueous manual. Samples were eluted using 2×25 μl 95° C. Elution Solution. Two, 1.5 μl DNase treatments were performed for one hr each at 37° C. using DNA-free (Ambion) to remove any genomic contamination. Twenty μl of purified RNA was used in 40 μl final volume RT reaction with heat denaturation as described in RETROscript (Ambion) protocol to generate cDNA. Samples were denatured at 85° C., and reverse transcribed by incubating for one hour at 42° C., followed by a ten minute incubation at 92° C.

Quantitative PCR was performed using primers and probes, specific to each ORF, designed using Primer Express software (Applied Biosystems, Foster City, Calif., USA). Twenty-five μl reactions were set up using 2× Taqman Universal PCR Master Mix (Applied Biosystems), 300 nM forward primer, 300 nM reverse primer, 200 nM FAM/TAMRA probe, and cDNA template. PCR reaction was as follows: 50° C. for 2 min, 95° C. for 10 min, 40 cycles of 95° C. for 15 seconds and 60° C. for one minute. Ribosomal 16S RNA is used as an internal control, with all results being normalized to the 16S Ct value. Based upon results from a standard curve, the cDNA added to these wells was diluted 100 fold to produce a Ct value similar to ORFs of interest.

Purification of Human Polymorphonuclear Leukocytes (PMN)

PMNs were purified from a pool of human whole blood from four donors using a Percoll gradient. A three-layer gradient was prepared by diluting Percoll in Hank's Balanced Salt Solution (HBSS). The densest phase was 2.7:1, middle was 1.079:1 and upper phase 1.07:1, Percoll:HBSS respectively. A ten ml volume of whole blood was layered onto the gradient and centrifuged at 2600 RPM for 20 minutes at 20° C. The upper layers were removed, washed in PBS with glucose to remove Percoll, centrifuged and resuspended in sterile water to lyse red blood cells. A twenty-fold concentrated solution of normal saline was added to equilibrate, re-centrifuged to remove lysed cells, the PMNs were resuspended and counted. The cells were diluted into PBS containing calcium and magnesium and brought to 37° C. before use.

Blot Analysis of ORF Specific Antibodies from Human Sera

Two μg of protein were coated onto nitrocellulose and allowed to air dry for 15 minutes. The blot was incubated in BLOTTO for 30 minutes at room temperature and then incubated with 5 ml of pooled human serum plasma at 4° C. for 16 hours. The nitrocellulose was rinsed in PBS with 0.2% Tween 20 and incubated with goat anti-human IgG conjugated to alkaline phosphatase for two hr at room temperature. The blot was re-washed and developed in NBT/BCIP substrate.

Affinity Purification of Human Antibodies

One hundred μg of each S. pyogenes purified protein was allowed to adhere to a strip of nitrocellulose, blocked for 15 minutes with 5% BLOTTO and then rinsed with PBS. After the sera was adsorbed overnight at 4° C., the nitrocellulose strip was washed with PBS and rinsed with 100 mM glycine at pH 3.0 to elute bound antibodies. The eluted antibodies were neutralized with 1 M Tris pH 8.8 and dialyzed in PBS. These antibodies were tested with PMNs and human whole blood for OPA to the SF-370 strain.

Opsonophagocytic Assay (OPA)

S. pyogenes strain SF-370 was used to inoculate THY broth and grown static overnight. The overnight cultures were diluted into fresh medium and further cultured to an OD₆₅₀ of 0.5-0.7. The cells were centrifuged, washed 1× with PBS and resuspended in ice cold PBS to an OD₆₅₀ of 0.5. The cells were diluted to 1:5,000 in PBS and mixed with test antibody or antiserum for 30 min at 4° C. Pre-warmed PMNs were added to the bacteria and antibody at a ratios of 100 and 200 effector cells per target cell. The reactions were incubated at 37° C. for one hr on a rocker and finally stopped with ice cold PBS and plated in duplicate on BHI agar.

OPA Using Whole Human Blood

Individual heparin-treated human blood was obtained and incubated at 37° C. for 15-30 min until used. Bacteria were prepared as described, and incubated with 50 μl test antibody at 4° C. for 15 min, then 430 μl of whole blood were added. The reactions were incubated for 1.5 hr at 37° C. on rocker and plated in duplicate on BHI agar. Each experiment represents an individual person's whole blood sample, not a pool.

### RESULTS

Whole Cell ELISA

The ability of ORF-specific antibody to react to the surface of whole cells was tested by ELISA. The antibody was produced in mice as described previously. Reactivity demonstrates differences in the amount of protein expressed on the surface of the S. pyogenes cells and/or the exposure of the protein in a manner that allows for antibody to bind. ELISA titers are shown in Table XV and indicate a range of reactivities reflective of the differences in either amount of protein expressed or number of epitopes exposed to allow for antibody reactivity. Values well above preimmune background titers are in bold face type.

TABLE XV
Whole cell ELISA titer to S. pyogenes ORFs.

| Orf # | ELISA Titer |
|---|---|
| 68 | 1,635 |
| 73 | 1,702 |
| 145 | 2,105 |
| 218 | 1,139 |
| 232 | 1,277 |
| 309 | 1,456 |
| 347 | 2,766 |
| 433 | 1,431 |
| 554 | 22,873 |
| 661 | 1,727 |
| 668 | 1,869 |
| 678 | 2,144 |
| 685 | 3,094 |
| 704 | 1,716 |
| 721 | 680 |
| 729 | 1,381 |
| 747 | 11,733 |
| 850 | 4,861 |
| 967 | 4,823 |
| 1157 | 1,827 |
| 1191 | 1,248 |
| 1202b | 1,194 |
| 1218 | 220,289 |
| 1224 | 21,170 |
| 1284 | 1,374 |
| 1316 | 6,407 |
| 1358 | 6,201 |
| 1487 | 4,007 |
| 1659 | 3,240 |
| 1664 | 5,355 |
| 1698 | 2,032 |
| 1723 | 1,273 |
| 1788 | 3,324 |
| 1789 | 1,475 |
| 1818 | 40,271 |
| 1820 | 2,498 |
| 1878 | 895 |
| 1983 | 1,179 |
| 2015 | 1,800 |
| 2019 | 24,669 |
| 2064 | 1,486 |
| 2258 | 4,962 |
| 2379 | 19,220 |
| 2417 | 4,225 |
| 2450 | 4,255 |
| 2452 | 2,256 |
| 2459 | 2,166 |
| 2477 | 5,412 |
| 2497 | 666 |
| 2593 | 8,602 |
| 2601 | 2,000 |

Gene Conservation

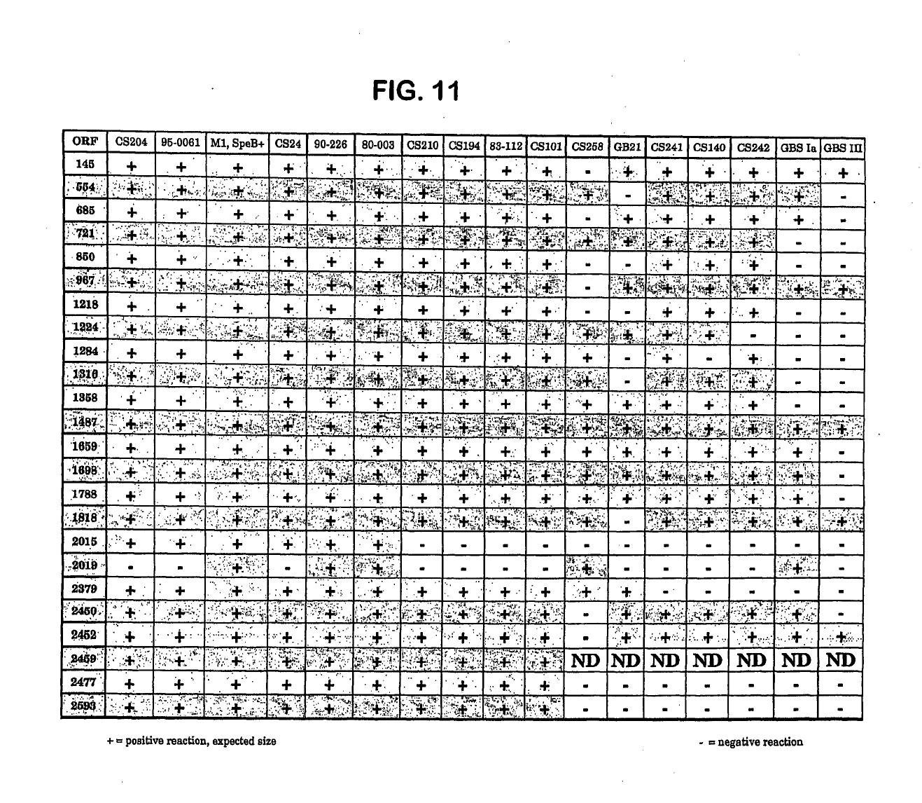

PCR analysis of several streptococcal strains was performed to determine the extent of conservation of the various ORFs. The results from this analysis can be seen in FIG. 11. All PCR products were analyzed by gel electrophoresis and the band size compared to the predicted value. All ORFs indicated as positive showed a PCR product migrating at the predicted size. The data show a high degree of genomic conservation, with 21 out of 24 ORFs tested being conserved across all eleven strains of S. pyogenes. Additionally, 18 were conserved amongst groups C and G; the lowest amount of conservation was observed in the strains of group B streptococci.

Quantitative PCR of Selected S. pyogenes ORFs

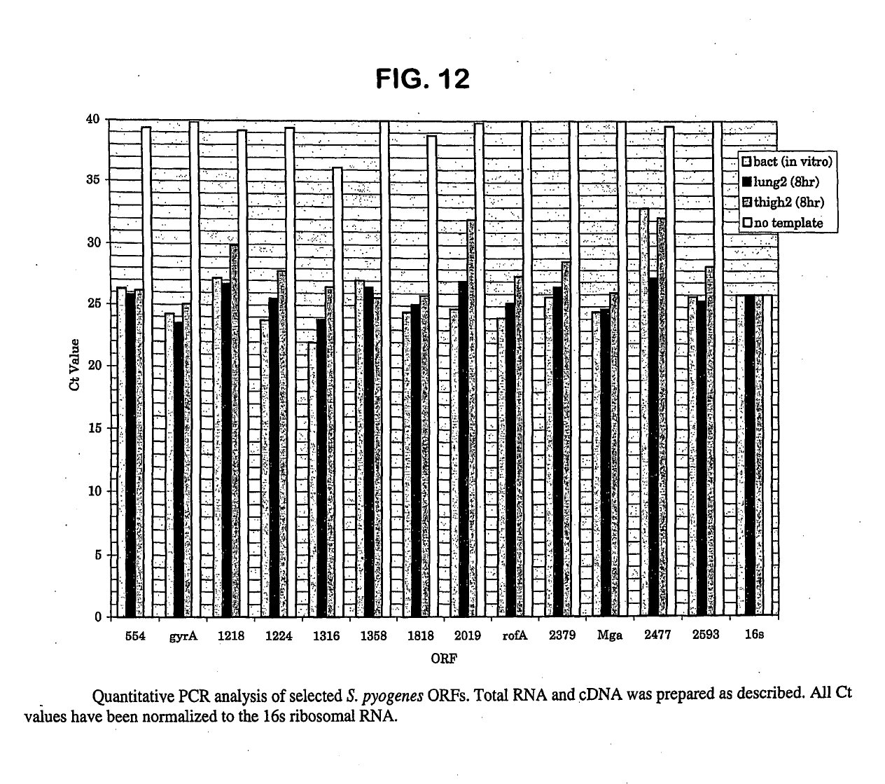

Quantitative PCR was performed to verify transcription of several ORFs contained in the S. pyogenes genome. Further, this method was used as a means to verify gene expression in vivo in a simulated infection model. Two known transcriptional regulators, rofA and Mga, and one other housekeeping gene, gyrA, were included as additional controls. All genes tested were expressed, and depending on conditions, some showed a variation in levels of transcription. The values are expressed in Ct numbers, which indicate at which PCR cycle the amplification was detectable above background. Thus, a lower Ct value indicates that a greater amount of MRNA was present in the starting material. A Ct difference of one correlates to a two-fold difference in the amount mRNA detected. FIG. 12 shows the results of this analysis. All ORFs showed a significantly lower Ct value than the no template control. ORF 2019 showed a 155-fold lower expression in the thigh than that observed in either the lung or in vitro culture. ORF 2477, on the other hand, showed a 49-fold increase, relative to the thigh or in vitro culture, in MRNA levels when extracted from the lung after 8 hours of infection. These data show that all ORFs tested were transcribed in vitro and in vivo and were influenced by the conditions in which the bacteria are exposed.

Reactivity of Human Sera to S. pyogenes Proteins

Antibodies were purified from human sera to test the ability of ORF specific antibody to enhance the ability of PMNs to engulf and kill S. pyogenes. Figure shows the reactivity of human serum to several S. pyogenes proteins by dot blot indicating that this serum is suitable as a source of antibodies for opsonophagocytic studies. Table XVI summarizes the results of these blots. The results of the blot indicate that 14 of the 24 ORF proteins tested positive for reactivity with human serum. In a similar experiment, a single human serum was tested against the proteins and the results were identical to the ones shown in Table XVI. Several of the proteins were selected for use in the affinity purified antibody studies based on their reactivity and quantity of available material.

TABLE XVI
ORF identification for reactive proteins.

| | A | B | C | D | E | F | G | H |
|---|---|---|---|---|---|---|---|---|
| 1 | ScpA | 145 | 232 | 554 | 668 | 721 | 1224 | 1284 |
| 2 | 2452 | 1659 | 1698 | 1788 | 1818 | 1820 | 2379 | 2459 |
| 3 | 2477 | 2593 | 2601 | 1218 | 433 | 1358 | 2019 | 1664 |

Notes:
Bold = positive

Opsonophagocytic Activity of Affinity Purified Human Anti-ORF Antibodies with Purified PMNs

PMNs were purified from a pool of four human blood samples and the growth of S. pyogenes SF-370 were as described above. Bacteria, PBS diluent and PMNs served as a negative control. The percent killing was calculated by dividing CFUs recovered from reaction containing test antibody with CFUs recovered from the reaction containing that of the negative control. The results of these studies, summarized in Table XVII, indicate that the affinity-purified antibodies have opsonic activity to SF-370 when incubated with purified PMNs. In particular, antibodies to ScpA and ORF 1224 resulted in greater than 50% killing as measured in OPA verses negative control all three times they were tested.

TABLE XVII
Opsonophagocytic activity of affinity purified human antibodies
to S. pyogenes proteins with purified PMNs as effector cells.

| | Opsonophagocytic Killing of ORF Antibodies (Percent)¹ | | | | | |
|---|---|---|---|---|---|---|
| | ScpA | 1224 | 1218 | 145 | 2459 | 1698 |
| Exp. #1 | 60 | 64 | 63 | ND | ND | ND |
| Exp. #2 | 65 | 53 | 59 | ND | ND | ND |
| Exp. #3 | 62 | 85 | 45 | 71 | 31 | 61 |
| Avg. | 62.3 | 67.3 | 55.7 | 71 | 31 | 61 |

¹Opsonophagocytic activity as compared to negative control. Ratio of PMNs to bacteria was 100:1. Affinity purified antibody was 10% of the reaction mixture (1:10 dilution).
ND = No data.

Opsonophagocytic Activity of Affinity Purified Hyman Antibodies Using Whole Blood

Traditional OPAs with S. pyogenes have utilized whole blood as the source of effector cells. Experiments were conducted to determine if the affinity-purified antibodies had opsonic activity in the presence of whole blood. The results are summarized in Table XVIII and show variable results depending on the individual whose blood was used as a source for PMNs. However, antibodies to ORF1224 and 145 gave consistently greater OPA titers with all seven of the individual blood samples tested. In contrast, antibodies to ScpA generated consistently poor OPA titers with all seven blood samples. This was unexpected because when antibodies to ScpA were tested with PMNs there was greater than 50% killing in 3 of 3 assays. Antibodies to the five other proteins had less consistent OPA against S. pyogenes SF-370 to the homologous strain. It should be noted that antibodies to ORF 1284 generated greater than 50% killing in 4 of 7 experiments.

TABLE XVIII
OPA using whole blood as source of effector cells.

| | Opsonophagocytic Killing of ORF Antibodies (Percent)¹ | | | | | | | |
|---|---|---|---|---|---|---|---|---|
| Person | ScpA | 145 | 1224 | 1284 | 1698 | 1818 | 2459 | 1218 |
| 1 | 16 | 77 | 86 | 60 | 56 | 45 | 82 | 56 |
| 2 | 36 | 50 | 79 | 86 | 68 | 72 | 64 | 28 |
| 3 | 16 | 47 | 56 | 53 | 39 | 42 | 66 | 33 |
| 4 | 14 | 48 | 54 | 41 | 25 | 63 | 62 | 33 |
| 5 | 19 | 69 | 56 | 35 | 63 | 42 | 19 | 42 |
| 6 | 7 | 57 | 68 | 54 | 62 | 54 | 65 | 36 |
| 7 | 5 | 64 | 59 | 42 | 33 | 38 | 19 | 16 |
| Mean | 14 | 58 | 64 | 51 | 32 | 50 | 47 | 33 |
| Std Dev | 10 | 12 | 13 | 17 | 20 | 13 | 25 | 12 |

¹Opsonophagocytic activity as compared to reaction containing whole blood, bacteria and PBS.

### EXAMPLE 4

### Biological Activities of Streptococcal Pyrogenic Exotoxin I

A study was undertaken to characterize SPE I with regard to biological activities. The data indicate that SPE I has superantigen activity and nonspecifically induces proliferation of T cells displaying T cell receptor Vβ regions (TCR Vβ) 6.7, 9, and 21.3.

SPE I

SPE I was purified by combinations of isoelectric focusing and affinity chromatography. The purified toxin was shown to be homogeneous by sodium dodecyl sulfate polyacrylamide gel electrophoresis.

Superantigenicity Assay

Rabbit splenocytes were seeded into the wells of a 96 well microtiter plate at a concentration of 2×10⁵ cells per well. Ten fold dilutions of toxin were added to wells in quadruplicate, starting with 1.0 μg/well down to 10⁻⁸ μg/well. These dilutions were compared to cells incubated in the presence of PBS alone as a negative control and other SPEs as positive controls. The splenocytes were grown at 37° C. for 3 days, and pulsed with 1 uCi ³H-thymidine overnight. The cells were harvested the next day, and cell proliferation, as determined by ³H-thymidine incorporation into DNA, was measured in a scintillation counter (Beckman Instruments, Fullerton, Calif.).

Flow Cytometric Analysis of T cell Repertoire

Peripheral blood mononuclear cells (PBMC) obtained from 3 normal human donors were isolated from heparinized venous blood by density gradient sedimentation over Ficoll-Hypaque (Histopaque, Sigma). Cells were then washed three times in Hank's balanced salt solution (HBSS) (Mediatech Cellgro, Herndon, Va.) and resuspended in medium for cell culture. PBMC (at 1×10⁶ cells/ml) were cultured in RPMI 1640 (Mediatech Cellgro) supplemented with 10% heat inactivated fetal calf serum (FCS) (Gemini Bioproducts, Woodland, Calif.), 20 mM HEPES buffer (Mediatech Cellgro), 100 u/ml penicillin (Mediatech Cellgro), 100 ug/ml streptomycin (Mediatech Cellgro), and 2 mM L glutamine (Mediatech Cellgro). Cells were cultured in the presence of either anti-CD3 (20 ng/ml), or SPE I (100 ng/ml) for 3 days, washed and allowed to grow for an additional day in the presence of interleukin 2 (50 U/ml) before washing and staining for immunofluoresence analysis of T cell repertoire as previous described.

For flow cytometry studies, PBMC were washed in HBSS and resuspended at 10×10⁶ cells/ml in a staining solution [PBS with 5% FCS (Gemini Bioproducts), 1% immunoglobulin (Alpha Therapeutic Corp., Los Angeles, Calif.), 0.02% sodium azide (Sigma)]. Cells were stained in 96 well, round bottomed plates with a panel of biotinylated monoclonal antibodies against human TCRVβ 2, 3, 5.1, 5.2, 7, 8, 11, 12, 13.1, 13.2, 14, 16, 17, 20, 21.3, 22 (Immunotech, Westbrook, Me.), TCRVβ 9, 23 (Pharmingen, San Diego, Calif.) and TCRVβ 6.7 fluorescein isothiocyanate (FITC) (Endogen, Woburn, Mass.), then incubated for 30 min at 37° C. in the dark. After the incubation period, cells were washed twice with washing buffer [PBS, 2% FCS (Gemini Bioproducts), 0.02% sodium azide (Sigma)] by centrifugation at 300× g for 5 min at 4° C. Cell pellets were resuspended in staining solution and incubated with anti-CD3 allophycocyanin (APC), anti-CD4 phycoerythrin (PE) (Becton Dickinson, San Jose, Calif.), anti-CD8 (FITC) (Becton Dickinson) and a streptavidin peridinin chlorophyll protein (PerCP) conjugate (Becton Dickinson) for 30 min at 4° C. Stained cells were again washed twice in washing buffer and once in 0.02% sodium azide (Sigma) in PBS, by centrifugation at 300× g for 5 min at 4° C. Finally, the cells were fixed in 200 ul of 1% (v/v) formaldehyde (Polysciences, Warrington, Pa.) in PBS. Analysis was performed using four color flow cytometry (FACS Calibur, Becton Dickinson) as described previously. Methods of cytometer set up and data acquisition have also been described previously. List mode multiparameter data files (each file with forward scatter, side scatter, and 4 fluorescent parameter) were analyzed using the Cellquest program (Becton Dickinson). Analysis of activated populations was performed with the light scatter gate set on the T cell blast population. Negative control reagents were used to verify the staining specificity of experimental antibodies.

Miniosmotic Pumps

Six American Dutch belted rabbits in groups of 3 were implanted with subcutaneous miniosmotic pumps on the left flanks, containing 500 ug of SPE I or 200 ug of TSST-1. Lethality of the toxins was assessed over a period of 15 days.

### RESULTS

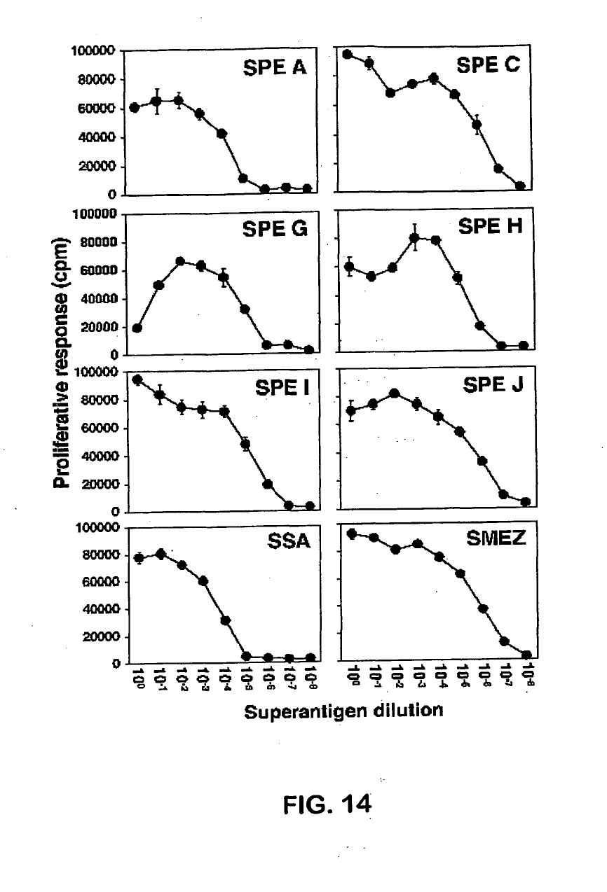

SPE I was evaluated for ability to induce rabbit splenocyte proliferation in a four day assay, as measured by incorporation of 3H thymidine into DNA (FIG. 14). SPE I was comparably mitogenic as the control SPE toxins also included in the figure. The complete fall-off of mitogenic activity for SPE I was between 10⁻⁶ and 10⁻⁷ ug/well, similar to that observed for other toxins.

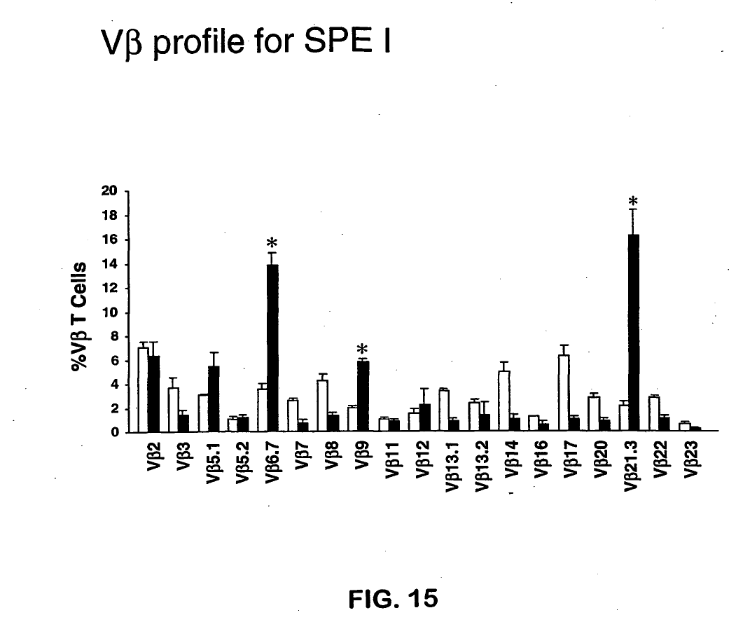

SPE I significantly stimulated human T cells bearing TCR Vβs 6.7, 9, and 21.3 (FIG. 15) compared to cells stimulated with anti-CD3 antibodies, consistent with SPE I being a superantigen. Some T cell populations, for example T cells with TCR Vβ 14 or 17 were significantly reduced compared to cells stimulated with anti-CD3 antibodies.

The majority of pyrogenic toxin superantigens are lethal when administered to rabbits at a toxin concentration between 200 and 500 ug in subcutaneously implanted miniosmotic pumps. SPE I did not exhibit this property at the 500 ug dose (3/3 survived). In contrast 200 ug of TSST-1 was completely lethal (3/3 succumbed).

### DISCUSSION

Pyrogenic toxin superantigens are defined by their abilities to induce T lymphocyte proliferation nonspecifically but dependent on the composition of the variable part of the beta chain of the T cell receptor (6). Thus for example, TSST-1 will stimulate proliferation of any human T cell bearing TCR Vβ2, without regard for the antigenic specificity of the responding T cells. This high level of stimulation leads to massive release of cytokines from both T cells and macrophages. Of particular importance is the release of tumor necrosis factors α and β that cause the hypotension and shock associated with TSS.

The data show that SPE I stimulates T cells as a superantigen. Thus, SPE I causes human peripheral blood mononuclear cells to proliferate that contain TCR Vβ6.7. 9, and 21.3. This elevation of these selected T cell populations, with the concurrent relative reduction of non-stimulated T cells, is the hallmark signal of SPE I and is referred to as Vβ skewing.

In addition, many pyrogenic toxin superantigens are lethal when administered to rabbits in subcutaneously implanted miniosmotic pumps, as a model for TSS (8). These pumps are designed to release a constant amount of toxin over a period of 7 days. The experiments continue for 15 days, however, since rabbits may succumb to the administered toxin for up to that period of time. SPE I was not lethal in this model of TSS. Although many pyrogenic toxin superantigens are lethal in this assay, there are notable exceptions. For example, the newly identified staphylococcal enterotoxins L and Q are not lethal in this model, yet these two toxins share all other activities expected of the family (including superantigenicity). For these latter toxins, it has been suggested that they either are not stable in the miniosmotic pumps for the entire 7 day toxin release period or precipitate in the pumps. Accordingly, SPE I shares defining superantigenic property of pyrogenic toxin superantigens.

Although illustrated and described above with reference to specific embodiments, the invention is nevertheless not intended to be limited to the details shown. Rather, various modifications may be made in the details within the scope and range of equivalents of the claims and without departing from the spirit of the invention.

### BIBLIOGRAPHY

1. 1997. Case definitions for Infectious Conditions Under Public Health Surveillance. CDC.

2. Alm, R. A., L. S. Ling, D. T. Moir, B. L. King, E. D. Brown, P. C. Doig, D. R. Smith, B. Noonan, B. C. Guild, B. L. deJonge, G. Carmel, P. J. Tummino, A. Caruso, M. Uria-Nickelsen, D. M. Mills, C. Ives, R. Gibson, D. Merberg, S. D. Mills, Q. Jiang, D. E. Taylor, G. F. Vovis, and T. J. Trust. 1999. Genomic-sequence comparison of two unrelated isolates of the human gastric pathogen Helicobacter pylori [published erratum appears in Nature 1999 Feb. 25; 397(6721):719]. Nature. 397:176-80.

3. Altschul, S. F., T. L. Madden, A. A. Schaffer, J. Zhang, Z. Zhang, W. Miller, and D. J. Lipman. 1997. Gapped BLAST and PSI-BLAST: a new generation of protein database search programs. Nucleic Acids Res. 25:3389-402.

4. Anderson, T. F. 1951. Techniques for the preservation of three-dimensional structure in preparing specimens for the electron microscope. Trans N Y Acad Sci. 13:130-134.

5. Benson, G. 1999. Tandem repeats finder: a program to analyze DNA sequences. Nucleic Acids Res. 27:573-80.

6. Chen, C. C., and P. P. Cleary. 1989. Cloning and expression of the streptococcal C5a peptidase gene in Escherichia coli: linkage to the type 12 M protein gene. Infect. Immun. 57:1740-1745.

7. Chmouryguina, I., A. Suvorov, P. Ferrieri, and P. P. Cleary. 1996. Conservation of the C5a peptidase genes in group A and B streptococci. Infect. Immun. 64:2387-2390.

8. Cockerill, F. R., 3rd, R. L. Thompson, J. M. Musser, P. M. Schlievert, J. Talbot, K. E. Holley, W. S. Harmsen, D. M. Ilstrup, P. C. Kohner, M. H. Kim, B. Frankfort, J. M. Manahan, J. M. Steckelberg, F. Roberson, and W. R. Wilson. 1998. Molecular, serological, and clinical features of 16 consecutive cases of invasive streptococcal disease. Southeastern Minnesota Streptococcal Working Group. Clin Infect Dis. 26:1448-58.

9. Courtney, H. S., Y. Li, J. B. Dale, and D. L. Hasty. 1994. Cloning, sequencing, and expression of a fibronectin/fibrinogen-binding protein from group A streptococci. Infect Immun. 62:3937-46.

10. Cserzo, M., E. Wallin, I. Simon, G. von Heijne, and A. Elofsson. 1997. Prediction of transmembrane alpha-helices in prokaryotic membrane proteins: the dense alignment surface method. Protein Engineering. 10:673-6.

11. Cunningham, M. W., and A. Quinn. 1997. Immunological crossreactivity between the class I epitope of streptococcal M protein and myosin. Adv Exp Med Biol. 418:887-92.

12. Dale, J. B., R. W. Baird, H. S. Courtney, D. L. Hasty, and M. S. Bronze. 1994. Passive protection of mice against group A streptococcal pharyngeal infection by lipoteichoic acid. J Infect Dis. 169:319-23.

13. Dale, J. B., M. Simmons, E. C. Chiang, and E. Y. Chiang. 1996. Recombinant, octavalent group A streptococcal M protein vaccine. Vaccine. 14:944-8.

14. Dale, J. B., R. G. Washburn, M. B. Marques, and M. R. Wessels. 1996. Hyaluronate capsule and surface M protein in resistance to opsonization of group A streptococci. Infect Immun. 64:1495-501.

15. Eddy, S. R. 1996. Hidden Markov models. Cur Opin Struct Bio. 6:361-5.

16. Ellen, R. P., and R. J. Gibbons. 1972. M protein-associated adherence of Streptococcus pyogenes to epithelial surfaces: prerequisite for virulence. Infect Immun. 5:826-830.

17. Eng, J. K., A. L. McCormack, and J. R. Yates, 3rd. 1994. An approach to correlate tandem mass-spectral data of peptides with amino-acid-sequences in a protein database. Am Soc Mass Spectrometry. 5:976-89.

18. Fischetti, V. A., V. Pancholi, and O. Schneewind. 1990. Conservation of a hexapeptide sequence in the anchor region of surface proteins from gram-positive cocci. Mol Microbiol. 4:1603-5.

19. Fogg, G. C., and M. G. Caparon. 1997. Constitutive expression of fibronectin binding in Streptococcus pyogenes as a result of anaerobic activation of rofA. J Bacteriol. 179:6172-80.

20. Foster, T. J., and M. Hook. 1998. Surface protein adhesins of Staphylococcus aureus. Trends Microbiol. 6:484-8.

21. Fraser, C. M., S. Casjens, W. M. Huang, G. G. Sutton, R. Clayton, R. Lathigra, O. White, K. A. Ketchum, R. Dodson, E. K. Hickey, M. Gwinn, B. Dougherty, J. F. Tomb, R. D. Fleischmann, D. Richardson, J. Peterson, A. R. Kerlavage, J. Quackenbush, S. Salzberg, M. Hanson, R. van Vugt, N. Palmer, M. D. Adams, J. Gocayne, J. C. Venter, and et al. 1997. Genomic sequence of a Lyme disease spirochaete, Borrelia burgdoreri [see comments]. Nature. 390:580-6.

22. Hacker, J., G. Blum-Oehler, I. Muhldorfer, and H. Tschape. 1997. Pathogenicity islands of virulent bacteria: structure, function and impact on microbial evolution. Mol Microbiol. 23:1089-97.

23. Hanski, E., and M. Caparon. 1992. Protein F, a fibronectin-binding protein, is an adhesion of the group A streptococcus Streptococcus pyogenes. Proc Natl Acad Sci., USA. 89:6172-76.

24. Hanski, E., P. A. Horwitz, and M. G. Caparon. 1992. Expression of protein F, the fibronectin-binding protein of Streptococcus pyogenes JRS4, in heterologous streptococcal and enterococcal strains promotes their adherence to respiratory epithelial cells. Infect Immun. 60:5119-5125.

25. Hernandez-Sanchez, J., J. G. Valadez, J. V. Herrera, C. Ontiveros, and G. Guarneros. 1998. lambda bar minigene-mediated inhibition of protein synthesis involves accumulation of peptidyl-tRNA and starvation for tRNA. EMBO Journal. 17:3758-65.

26. Huang, T. T., H. Malke, and J. J. Ferretti. 1989. The streptokinase gene of group A streptococci: cloning, expression in Escherichia coli, and sequence analysis. Mol Microbiol. 3:197-205.

27. Hynes, W. L., A. R. Dixon, S. L. Walton, and L. J. Aridgides. 2000. The extracellular hyaluronidase gene (hylA) of Streptococcus pyogenes. FEMS Microbiol Lett. 184:109-12.

28. Hynes, W. L., L. Hancock, and J. J. Ferretti. 1995. Analysis of a second bacteriophage hyaluronidase gene from Streptococcus pyogenes: evidence for a third hyaluronidase involved in extracellular enzymatic activity. Infect Immun. 63:3015-20.

29. Isberg, R. R., and G. Tran Van Nhieu. 1994. Binding and internalization of microorganisms by integrin receptors. Trends Microbio. 2:10-4.

30. Jones, K. F., and V. A. Fischetti. 1988. The importance of the location of antibody binding on the M6 protein for opsonization and phagocytosis of group A M6 streptococci. J Exp Med. 167:1114-23.

31. Kihlberg, B. M., M. Collin, A. Olsen, and L. Bjorck. 1999. Protein H, an antiphagocytic surface protein in Streptococcus pyogenes. Infect Immun. 67:1708-14.

32. Koebnik, R. 1995. Proposal for a peptidoglycan-associating alpha-helical motif in the C-terminal regions of some bacterial cell-surface proteins [letter; comment]. Molecular Microbiology. 16:1269-70.

33. Loessner, M. J., S. Gaeng, and S. Scherer. 1999. Evidence for a holin-like protein gene fully embedded out of frame in the endolysin gene of Staphylococcus aureus bacteriophage 187. J Bacteriol. 181:4452-60.

34. Lukashin, A. V., and M. Borodovsky. 1998. GeneMark.hmm: new solutions for gene finding. Nucleic Acids Res. 26:1107-15.

35. Lukomski, S., C. A. Montgomery, J. Rurangirwa, R. S. Geske, J. P. Barrish, G. J. Adams, and J. M. Musser. 1999. Extracellular cysteine protease produced by Streptococcus pyogenes participates in the pathogenesis of invasive skin infection and dissemination in mice. Infect Immun. 67:1779-88.

36. Madore, D. V. 1998. Characterization of immune response as an indicator of Haemophilus influenzae type b vaccine efficacy. Pediatr Infect Dis J. 17:S207-10.

37. Matsuka, Y. V., S. Pillai, S. Gubba, J. M. Musser, and S. B. Olmsted. 1999. Fibrinogen cleavage by the Streptococcus pyogenes extracellular cysteine protease and generation of antibodies that inhibit enzyme proteolytic activity. Infect Immun. 67:4326-33.

38. Mazmanian, S. K., G. Liu, H. Ton-That, and O. Schneewind. 1999. Staphylococcus aureus sortase, an enzyme that anchors surface proteins to the cell wall. Science. 285:760-3.

39. McAtee, C. P., K. E. Fry, and D. E. Berg. 1998. Identification of potential diagnostic and vaccine candidates of Helicobacter pylori by “proteome” technologies. Helicobacter. 3:163-9.

40. McAtee, C. P., M. Y. Lim, K. Fung, M. Velligan, K. Fry, T. Chow, and D. E. Berg. 1998. Identification of potential diagnostic and vaccine candidates of Helicobacter pylori by two-dimensional gel electrophoresis, sequence analysis, and serum profiling. Clin Diagn Lab Immunol. 5:537-42.

41. McAtee, C. P., M. Y. Lim, K. Fung, M. Velligan, K. Fry, T. P. Chow, and D. E. Berg. 1998. Characterization of a Helicobacter pylori vaccine candidate by proteome techniques. J Chromatogr B Biomed Sci Appl. 714:325-33.

42. Mejlhede, N., J. F. Atkins, and J. Neuhard. 1999. Ribosomal-1 frameshifting during decoding of Bacillus subtilis cdd occurs at the sequence CGA AAG. J. Bacteriol. 181:2930-7.

43. Molinari, G., S. R. Talay, P. Valentin-Weigand, M. Rohde, and G. S. Chhatwal. 1997. The fibronectin-binding protein of Streptococcus pyogenes, SfbI, is involved in the internalization of group A streptococci by epithelial cells. Infect Immun. 65:1357-63.

44. Nakai, K., and M. Kanehisa. 1991. Expert system for predicting protein localization sites in gram-negative bacteria. Proteins. 11:95-110.

45. Navarre, W. W., and O. Schneewind. 1999. Surface proteins of gram-positive bacteria and mechanisms of their targeting to the cell wall envelope. Microbiol Mol Biol Rev. 63:174-229.

46. Nielsen, H., J. Engelbrecht, S. Brunak, and G. von Heijne. 1997. Identification of prokaryotic and eukaryotic signal peptides and prediction of their cleavage sites. Protein Engineering. 10:1-6.

47. Nizet, V., B. Beall, D. J. Bast, V. Datta, L. Kilburn, D. E. Low, and J. C. De Azavedo. 2000. Genetic locus for streptolysin S production by group A streptococcus. Infect Immun. 68:4245-54.

48. Nordstrand, A., W. M. McShan, J. J. Ferretti, S. E. Holm, and M. Norgren. 2000. Allele substitution of the streptokinase gene reduces the nephritogenic capacity of group A streptococcal strain NZ131. Infect Immun. 68:1019-25.

49. Olmsted, S. B., S. L. Erlandsen, G. M. Dunny, and C. L. Wells. 1993. High-resolution visualization by field emission scanning electron microscopy of Enterococcus faecalis surface proteins encoded by the pheromone-inducible conjugative plasmid pCF10. J Bacteriol. 175:6229-37.

50. Park, J., and S. A. Teichmann. 1998. DIVCLUS: an automatic method in the GEANFAMMER package that finds homologous domains in single- and multi-domain proteins. Bioinformatics. 14:144-50.

51. Parkhill, J., M. Achtman, K. D. James, S. D. Bentley, C. Churcher, S. R. Klee, G. Morelli, D. Basham, D. Brown, T. Chillingworth, R. M. Davies, P. Davis, K. Devlin, T. Feltwell, N. Hamlin, S. Holroyd, K. Jagels, S. Leather, S. Moule, K. Mungall, M. A. Quail, M. A. Rajandream, K. M. Rutherford, M. Simmonds, J. Skelton, S. Whitehead, B. G. Spratt, and B. G. Barrell. 2000. Complete DNA sequence of a serogroup A strain of Neisseria meningitidis Z2491 [see comments]. Nature. 404:502-6.

52. Pierschbacher, M. D., and E. Ruoslahti. 1987. Influence of stereochemistry of the sequence Arg-Gly-Asp-Xaa on binding specificity in cell adhesion. J Biol Chem. 262:17294-8.

53. Pizza, M., V. Scarlato, V. Masignani, M. M. Giuliani, B. Arico, M. Comanducci, G. T. Jennings, L. Baldi, E. Bartolini, B. Capecchi, C. L. Galeotti, E. Luzzi, R. Manetti, E. Marchetti, M. Mora, S. Nuti, G. Ratti, L. Santini, S. Savino, M. Scarselli, E. Storni, P. Zuo, M. Broeker, E. Hundt, B. Knapp, E. Blair, T. Mason, H. Tettelin, D. W. Hood, A. C. Jeffries, N. J. Saunders, D. M. Granoff, J. C. Venter, E. R. Moxon, G. Grandi, and R. Rappuoli. 2000. Identification of vaccine candidates against serogroup B meningococcus by whole-genome sequencing [see comments]. Science. 287:1816-20.

54. Podbielski, A., A. Flosdorff, and J. Weber-Heynemann. 1995. The group A streptococcal virR49 gene controls expression of four structural vir regulon genes. Infect Immun. 63:9-20.

55. Proft, T., S. Louise Moffatt, C. J. Berkahn, and J. D. Fraser. 1999. Identification and Characterization of Novel Superantigens from Streptococcus pyogenes. J Exp Med. 189:89-102.

56. Pugsley, A. P. 1993. The complete general secretory pathway in gram-negative bacteria. Microbiol Rev. 57:50-108.

57. Quinn, A., K. Ward, V. A. Fischetti, M. Hemric, and M. W. Cunningham. 1998. Immunological relationship between the class I epitope of streptococcal M protein and myosin. Infect Immun. 66:4418-24.

58. Reda, K. B., V. Kapur, D. Goela, J. G. Lamphear, J. M. Musser, and R. R. Rich. 1996. Phylogenetic distribution of streptococcal superantigen SSA allelic variants provides evidence for horizontal transfer of ssa within Streptococcus pyogenes. Infect Immun. 64:1161-5.

59. Salzberg, S. L., A. L. Delcher, S. Kasif, and O. White. 1998. Microbial gene identification using interpolated Markov models. Nucleic Acids Res. 26:544-8.

60. Sonnenberg, M. G., and J. T. Belisle. 1997. Definition of Mycobacterium tuberculosis culture filtrate proteins by two-dimensional polyacrylamide gel electrophoresis, N-terminal amino acid sequencing, and electrospray mass spectrometry. Infect Immun. 65:4515-24.

61. Bateman, A. T., R. Birney, S. P. Durbin, K. L. Howe and E. L. L. Sonnhammer. 2000. The Pfam protein families database. Nuc. Acids. Res. 28:263-6.

62. Stevens, D. L. 1995. Streptococcal toxic-shock syndrome: spectrum of disease, pathogenesis, and new concepts in treatment. Emerg Infect Dis. 1:69-78.

63. Stockbauer, K. E., L. Magoun, M. Liu, E. H. Burns, Jr., S. Gubba, S. Renish, X. Pan, S. C. Bodary, E. Baker, J. Coburn, J. M. Leong, and J. M. Musser. 1999. A natural variant of the cysteine protease virulence factor of group A streptococcus with an arginine-glycine-aspartic acid (RGD) motif preferentially binds human integrins alphavbeta3 and alphaIIbbeta3 [In Process Citation]. Proc Natl Acad Sci., USA. 96:242-7.

64. Ton-That, H., G. Liu, S. K. Mazmanian, K. F. Faull, and O. Schneewind. 1999. Purification and characterization of sortase, the transpeptidase that cleaves surface proteins of Staphylococcus aureus at the LPXTG motif. Proc Natl Acad Sci USA. 96:12424-12429.

65. Weldingh, K., I. Rosenkrands, S. Jacobsen, P. B. Rasmussen, M. J. Elhay, and P. Andersen. 1998. Two-dimensional electrophoresis for analysis of Mycobacterium tuberculosis culture filtrate and purification and characterization of six novel proteins. Infect Immun. 66:3492-500.

66. Yutsudo, T., K. Okumura, M. Iwasaki, A. Hara, S. Kamitani, W. Minamide, H. Igarashi, and Y. Hinuma. 1994. The gene encoding a new mitogenic factor in a Streptococcus pyogenes strain is distributed only in group A streptococci. Infection and Immunity. 62:4000-4004.

67. Published International Patent Application Number WO99/27944.

68. U.S Pat. No. 4,666,829.

What is claimed is:

1. An isolated polypeptide comprising:
(a) an amino acid sequence that has at least 70% identity to the amino acid sequence of SEQ ID NO: 140; or
(b) an amino acid sequence that is encoded by a nucleic acid sequence having at least 70% identity to the nucleic acid sequence of SEQ ID NO: 139;
wherein administration of the isolated polypeptide induces antibodies having opsonophagocytic activity of at least about 30 percent killing of bacteria as measured by decrease in colony forming units (CFU) in OPA versus a negative control.

2. The isolated polypeptide of claim 1, wherein administration of the isolated polypeptide induces antibodies having an opsonophagocytic activity of at least about 50% percent killing of bacteria as measured by decrease in colony forming units (CFU) in OPA versus a negative control.

3. The isolated polypeptide of claim 1, wherein the isolated polypeptide provides a desired level of protection against β-hemolytic streptococci.

4. The isolated polypeptide of claim 1, comprising an amino acid sequence that has at least 90% identity to an amino acid sequence of SEQ ID NO:140.

5. The isolated polypeptide of claim 1, comprising an amino acid sequence that has at least 95% identity to an amino acid sequence of SEQ ID NO:140.

6. The isolated polypeptide of claim 1, wherein the biological equivalent provides cross-reactivity across at least two strains of β-hemolytic streptococci.

7. The isolated polypeptide of claim 1, where said isolated polypeptide is the mature polypeptide.

8. An isolated polypeptide comprising:
(a) an amino acid sequence that comprises the amino acid sequence of SEQ ID NO: 140; or
(b) an amino acid sequence that is encoded by a nucleic acid sequence comprising the nucleic acid sequence of SEQ ID NO: 139.

9. An isolated polypeptide comprising:
an amino acid sequence that comprises at least 7 contiguous amino acid residues of the amino acid sequence of SEQ ID NO:140;
wherein administration of the isolated polypeptide induces antibodies having opsonophagocytic activity of at least about 30 percent killing of bacteria as measured by decrease in colony forming units (CFU) in OPA versus a negative control.

10. An isolated polynucleotide comprising:
(i) a nucleotide sequence that encodes an amino acid sequence that has at least 70% identity to the amino acid sequence of SEQ ID NO:140; or
(b) a nucleotide sequence that has at least 70% identity to the nucleic acid sequence of SEQ ID NO:139;
wherein the isolated polynucleotide encodes a polypeptide that exhibits opsonophagocytic activity of at least about 30 percent killing of bacteria as measured by decrease in colony forming units (CFU) in OPA versus a negative control.

11. An isolated polynucleotide comprising:
(i) a nucleotide sequence that encodes the isolated polypeptide of claim 1;
(ii) a nucleotide sequence that has at least 70% identity to a nucleotide sequence that encodes the isolated polypeptide of claim 1;
(iii) a nucleotide sequence that has at least 70% identity to the nucleotide sequence of SEQ ID NO:139;
(iv) a nucleotide sequence that encodes an amino acid sequence having at least 70% identify to the amino acid sequence of SEQ ID NO:140; or
(v) a nucleotide sequence that is fully complementary to a nucleotide sequence of any of (i)-(iv);
wherein administration of the isolated polypeptide induces antibodies having opsonophagocytic activity of at least about 30 percent killing of bacteria as measured by decrease in colony forming units (CFU) in OPA versus a negative control.

12. The isolated polynucleotide of claim 11, wherein the nucleotide sequence is SEQ ID NO:139.

13. The isolated polynucleotide of claim 11, where said isolated polypeptide is a mature polypeptide.

14. An isolated polynucleotide comprising:
(a) a nucleotide sequence that comprises the nucleic acid sequence of SEQ ID NO:139; or
(b) a nucleotide sequence that encodes an isolated polynucleotide comprising the amino acid sequence of SEQ ID NO:140.

15. A recombinant host cell comprising a polynucleotide of claim 11.

16. A recombinant expression vector comprising a polynucleotide of claim 11.

17. A recombinant host cell comprising a vector of claim 11.

18. A method for producing a polypeptide comprising:
(a) culturing a recombinant host cell comprising (i) a polynucleotide of claim 11 or (ii) a recombinant expression vector comprising a polynucleotide of claim 11, under conditions suitable to produce the polypeptide encoded by the polynucleotide; and
(b) recovering the polypeptide from the culture.

19. An antibody that binds immunospecifically to a polypeptide of claim 1.

20. The antibody of claim 19, wherein the antibody binds immunospecifically to a polypeptide having an amino acid sequence of SEQ ID NO:140.

21. An immunogenic composition comprising an immunogenic amount of a component that comprises a polypeptide of claim 1, wherein the polypeptide is capable of generating antibody that specifically recognizes said polypeptide, and wherein the amount of said component is effective to prevent or ameliorate β-hemolytic streptococcal colonization or infection in a susceptible mammal.

22. The immunogenic composition of claim 21, which comprises at least a portion of said polypeptide conjugated or linked to a peptide, polypeptide, or protein.

23. The immunogenic composition of claim 21, which comprises at least a portion of said polypeptide conjugated or linked to a polysaccharide.

24. The immunogenic composition of claim 21, which further comprises a physiologically-acceptable vehicle.

25. The immunogenic composition of claim 21, which further comprises an effective amount of an adjuvant.

26. An immunogenic composition comprising an immunogenic amount of a component that comprises a polynucleotide of claim 11, wherein said component is in an amount effective to prevent or ameliorate a β-hemolytic streptococcal colonization or infection in a susceptible mammal.

27. The immunogenic composition of claim 26, comprising a recombinant expression vector comprising a polynucleotide of claim 11.

28. The immunogenic composition of claim 26, wherein the β-hemolytic streptococci is group A streptococci, group B streptococci, group C streptococci, or group G streptococci.

29. The immunogenic composition of claim 28, wherein the β-hemolytic streptococci is Streptococcus pyogenes.

30. An immunogenic composition comprising:
(i) an isolated polypeptide that is substantially conserved across strains of β-hemolytic streptococci and that is effective in preventing or ameliorating a β-hemolytic streptococcal colonization or infection in a susceptible subject, said isolated polypeptide having at least 70% identity to the amino acid sequence of SEQ ID NO:140 ; or
(ii) an immunogenic fragment of (i).

31. The immunogenic composition of claim 30, wherein the β-hemolytic streptococci is group A streptococci, group B streptococci, group C streptococci, or group G streptococci.

32. The immunogenic composition of claim 30, wherein the β-hemolytic streptococci is Streptococcus pyogenes.

33. A method of protecting a susceptible mammal against β-hemolytic streptococcal colonization or infection comprising administering to the mammal an effective amount of an immunogenic composition comprising a polypeptide of claim 1, wherein the polypeptide is capable of generating antibody specific to said polypeptide, and wherein the amount is effective to prevent or ameliorate β-hemolytic streptococcal colonization or infection in the susceptible mammal.

34. The method of claim 33, wherein the immunogenic composition comprises at least a portion of said polypeptide, optionally conjugated or linked to a peptide, polypeptide, or protein.

35. The method of claim 33, wherein the immunogenic composition comprises at least a portion of said polypeptide, optionally conjugated or linked to a polysaccharide.

36. The method of claim 33, wherein the polypeptide comprises the mature polypeptide of an amino acid sequence of SEQ ID NO:140.

37. The method of claim 33, wherein the immunogenic composition further comprises a physiologically-acceptable vehicle.

38. The method of claim 33, wherein the immunogenic composition is administered by subcutaneous injection, by intramuscular injection, by oral ingestion, intranasally, or combinations thereof.

39. The method of claim 33, wherein the β-hemolytic streptococci is group A streptococci, group B streptococci, group C streptococci, or group G streptococci.

40. The method of claim 33, wherein the β-hemolytic streptococci is Streptococcus pyogenes.

41. A method of protecting a susceptible mammal against β-hemolytic streptococcal colonization or infection comprising administering to the mammal an effective amount of an immunogenic composition comprising a polynucleotide of claim 14, which amount is effective to prevent or ameliorate β-hemolytic streptococcal colonization or infection in the susceptible mammal.

42. The method of claim 41, wherein said immunogenic composition comprises a recombinant expression vector comprising the polynucleotide of claim 11.

43. The method of claim 41, wherein the immunogenic composition further comprises a physiologically-acceptable vehicle.

44. The method of claim 41, wherein the immunogenic composition is administered by subcutaneous injection, by intramuscular injection, by oral ingestion, intranasally, or combinations thereof.

45. The method of claim 41, wherein the β-hemolytic streptococci is group A streptococci, group B streptococci, group C streptococci, or group G streptococci.

46. The method of claim 41, wherein the β-hemolytic streptococci is Streptococcus pyogenes.

47. An isolated polypeptide comprising:
(i) an amino acid sequence that has at least 70% identity to an amino acid sequence of any of even numbered SEQ ID NOS: 2-668;
(ii) an amino acid sequence of any of even numbered SEQ ID NOS: 2-668;
(iii) an immunogenic fragment of any amino acid sequence of (i) or (ii);
(iv) at least 7 contiguous amino acid residues of any amino acid sequence of (i) or (ii); or
(v) a biological equivalent of any of (i), (ii), (iii) or (iv) that is effective for preventing or ameliorating β-hemolytic streptococcal colonization or infection in a susceptible subject.
